SEQUENCE LISTING (185 sequences)

SEQ ID NO: 1 - protein, 444 aa, Oryctolagus cuniculus
MIEDNKENKDHSLERGRATLIFSLKNEVGGLIKALKIFQEKHVNLLHIESRKSKRRNSEFEIFVDCDTNREQLNDIFHLLKSHTNVLSVTPPDNFTMKEEGMESVPWFPKKISDLDHCANRVLMYGSELDADHPGFKDNVYRKRRKYFADLAMSYKYGDPIPKVEFTEEEIKTWGTVFRELNKLYPTHACREYLKNLPLLSKYCGYREDNIPQLEDISNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHVLSGHAKVKPFDPKITYKQECLITTFQDVYFVSESFEDAKEKMREFTKTIKRPFGVKYNPYTRSIQILKDAKSITNAMNELRHDLDVVSDALGKVSRQLSV

SEQ ID NO: 2 - protein, 444 aa, Homo sapiens
MIEDNKENKDHSLERGRASLIFSLKNEVGGLIKALKIFQEKHVNLLHIESRKSKRRNSEFEIFVDCDINREQLNDIFHLLKSHTNVLSVNLPDNFTLKEDGMETVPWFPKKISDLDHCANRVLMYGSELDADHPGFKDNVYRKRRKYFADLAMNYKHGDPIPKVEFTEEEIKTWGTVFQELNKLYPTHACREYLKNLPLLSKYCGYREDNIPQLEDVSNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHALSGHAKVKPFDPKITCKQECLITTFQDVYFVSESFEDAKEKMREFTKTIKRPFGVKYNPYTRSIQILKDTKSITSAMNELQHDLDVVSDALAKVSRKPSI

SEQ ID NO: 3 - protein, 496 aa, Homo sapiens [sequence omitted]

SEQ ID NO: 4 - protein, 502 aa, Bos taurus
MQPAMMMFSSKYWARRGLSLDSAVPEEHQLLTSLTLNKTNSGKNDDKKGNKGSSKNDTATESGKTAVVFSLKNEVGGLVKALKLFQEKHVNMIHIESRKSRRRSSEVEIFVDCECGKTEFNELIQSLKFQTTIVTLNPPENIWTEEEGKLTCVAKGKELEDVPWFPRKISELDRCSHRVLMYGSELDADHPGFKDNVYRQRRKYFVDVAMGYKYGQPIPRVEYTEEETKTWGVVFRELSKLYPTHACREYLKNFPLLTKHCGYREDNVPQLEDVAAFLKERSGFTVRPVAGYLSPRDFLAGLAYRVFHCTQYVRHGSDPLYTPEPDVTLSLLSHVPLIFDDQFPTSFSNEVGRAVILASWGDKQENNQCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRSDLNTVCDALNKMNQYLGI

SEQ ID NO: 5 - protein, 497 aa, Sus scrofa
MQPAMMMFSSKYWARRGLSLDSAVPEEHQLLGSLTVSTFLKLNKSNSGKNDDKKGNKGSGKSDTATESGKTAVVFSLKNEVGGLVKALKLFQEKHVNMVHIESRKSRRRSSEVEIFVDCECGKTEFNELIQSLKFQTTIVTLNPPENIWTEEEELEDVPWFPRKISELDKCSHRVLMYGSELDADHPGFKDNVYRQRRKYFVDLAMGYKYGQPIPRVEYTEEETKTWGIVFRELSKLYPTHACREYLKNFPLLTKYCGYREDNVPQLEDVSVFLKERSGFTVRPVAGYLSPRDFLAGLAYRVFHCTQYVRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRSDLNTVCDALNKMNQYLGI

SEQ ID NO: 6 - protein, 445 aa, Gallus gallus
MIEDNKENKDHAPERGRTAIIFSLKNEVGGLVKALKLFQEKHVNLVHIESRKSKRRNSEFEIFVDCDSNREQLNEIFQLLKSHVSIVSMNPTEHFNVQEDGDMENIPWYPKKISDLDKCANRVLMYGSDLDADHPGFKDNVYRKRRKYFADLAMNYKHGDPIPEIEFTEEEIKTWGTVYRELNKLYPTHACREYLKNLPLLTKYCGYREDNIPQLEDVSRFLKERTGFTIRPVAGYLSPRDFLAGLAFRVFHCTQYVRHSSDPLYTPEPDTCHELLGHVPLLAEPSFAQFSQEIGLASLGASDEAVQKLATCYFFTVEFGLCKQEGQLRVYGAGLLSSISELKHSLSGSAKVKPFDPKVTCKQECLITTFQEVYFVSESFEEAKEKMREFAKTIKRPFGVKYNPYTQSVQILKDTKSIASVVNELRHELDIVSDALSKMGKQLEV

SEQ ID NO: 7 - protein, 447 aa, Mus musculus
MIEDNKENKENKDHSSERGRVTLIFSLENEVGGLIKVLKIFQENHVSLLHIESRKSKQRNSEFEIFVDCDISREQLNDIFPLLKSHATVLSVDSPDQLTAKEDVMETVPWFPKKISDLDFCANRVLLYGSELDADHPGFKDNVYRRRRKYFAELAMNYKHGDPIPKIEFTEEEIKTWGTIFRELNKLYPTHACREYLRNLPLLSKYCGYREDNIPQLEDVSNFLKERTGFSIRPVAGYLSPRDFLSGLAFRVFHCTQYVRHSSDPLYTPEPDTCHELLGHVPLLAEPSFAQFSQEIGLASLGASEETVQKLATCYFFTVEFGLCKQDGQLRVFGAGLLSSISELKHALSGHAKVKPFDPKIACKQECLITSFQDVYFVSESFEDAKEKMREFAKTVKRPFGLKYNPYTQSVQVLRDTKSITSAMNELRYDLDVISDALARVTRWPSV

SEQ ID NO: 8 - protein, 491 aa, Equus caballus
MQPAMMMFSSKYWARRGFSLDSAVPEEHQLLGNLTVNKSNSGKNDDKKGNKGSSRSETAPDSGKTAVVFSLRNEVGGLVKALKLFQEKHVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMSYKYGQPIPRVEYTEEETKTWGVVFRELSRLYPTHACQEYLKNFPLLTKYCGYREDNVPQLEDVSMFLKERSGFAVRPVAGYLSPRDFLAGLAYRVFHCTQYVRHSSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMREFAKSITRPFSVHFNPYTQSVEVLKDSRSIESVVQDLRSDLNTVCDALNKMNQYLGV

SEQ ID NO: 9 - protein, 410 aa, Homo sapiens
MDDKGNKGSSKREAATESGKTAVVFSLKNEVGGLVKALRLFQEKRVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRKALPVRLACILTRIPRALKF

SEQ ID NO: 10 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 11 - protein, 241 aa, Mus musculus
MEKPRGVRCTNGFSERELPRPGASPPAEKSRPPEAKGAQPADAWKAGRHRSEEENQVNLPKLAAAYSSILLSLGEDPQRQGLLKTPWRAATAMQYFTKGYQETISDVLNDAIFDEDHDEMVIVKDIDMFSMCEHHLVPFVGRVHIGYLPNKQVLGLSKLARIVEIYSRRLQVQERLTKQIAVAITEALQPAGVGVVIEATHMCMVMRGVQKMNSKTVTSTMLGVFREDPKTREEFLTLIRS

SEQ ID NO: 12 - protein, 222 aa, Escherichia coli
MPSLSKEAALVHEALVARGLETPLRPPVHEMDNETRKSLIAGHMTEIMQLLNLDLADDSLMETPHRIAKMYVDEIFSGLDYANFPKITLIENKMKVDEMVTVRDITLTSTCEHHFVTIDGKATVAYIPKDSVIGLSKINRIVQFFAQRPQVQERLTQQILIALQTLLGTNNVAVSIDAVHYCVKARGIRDATSATTTTSLGGLFKSSQNTRHEFLRAVRHHN

SEQ ID NO: 13 - protein, 243 aa, Saccharomyces cerevisiae
MHNIQLVQEIERHETPLNIRPTSPYTLNPPVERDGFSWPSVGTRQRAEETEEEEKERIQRISGAIKTILTELGEDVNREGLLDTPQRYAKAMLYFTKGYQTNIMDDVIKNAVFEEDHDEMVIVRDIEIYSLCEHHLVPFFGKVHIGYIPNKKVIGLSKLARLAEMYARRLQVQERLTKQIAMALSDILKPLGVAVVMEASHMCMVSRGIQKTGSSTVTSCMLGGFRAHKTREEFLTLLGRRSI

SEQ ID NO: 14 - protein, 190 aa, Bacillus subtilis
MKEVNKEQIEQAVRQILEAIGEDPNREGLLDTPKRVAKMYAEVFSGLNEDPKEHFQTIFGENHEELVLVKDIAFHSMCEHHLVPFYGKAHVAYIPRGGKVTGLSKLARAVEAVAKRPQLQERITSTIAESIVETLDPHGVMVVVEAEHMCMTMRGVRKPGAKTVTSAVRGVFKDDAAARAEVLEHIKRQD

SEQ ID NO: 15 - protein, 201 aa, Streptomyces avermitilis
MTDPVTLDGEGTIGEFDEKRAENAVRELLIAVGEDPDREGLRETPGRVARAYREIFAGLWQKPEDVLTTTFDIGHDEMVLVKDIEVLSSCEHHLVPFVGVAHVGYIPSTDGKITGLSKLARLVDVYARRPQVQERLTTQVADSLMEILEPRGVIVVVECEHMCMSMRGVRKPGAKTITSAVRGQLRDPATRNEAMSLIMAR

SEQ ID NO: 16 - protein, 222 aa, Salmonella typhi
MPSLSKEAALVHDALVARGLETPLRPPMDELDNETRKSLIAGHMTEIMQLLNLDLSDDSLMETPHRIAKMYVDEIFAGLDYANFPKITLIENKMKVDEMVTVRDITLTSTCEHHFVTIDGKATVAYIPKDSVIGLSKINRIVQFFAQRPQVQERLTQQILTALQTLLGTNNVAVSIDAVHYCVKARGIRDATSATTTTSLGGLFKSSQNTRQEFLRAVRHHP

SEQ ID NO: 17 - protein, 250 aa, Homo sapiens; identical to SEQ ID NO: 10

SEQ ID NO: 18 - protein, 144 aa, Rattus norvegicus
MNAAVGLRRRARLSRLVSFSASHRLHSPSLSAEENLKVFGKCNNPNGHGHNYKVVVTIHGEIDPVTGMVMNLTDLKEYMEEAIMKPLDHKNLDLDVPYFADVVSTTENVAVYIWENLQRLLPVGALYKVKVYETDNNIVVYKGE

SEQ ID NO: 19 - protein, 124 aa, Bacteroides thetaiotaomicron
MFTVIKRMEISASHKLVLPYRSKCASLHGHNWIITVYCRSSRLNSEGMVVDFTRIKEVVTEKLDHQNLNEVLPFNPTAENIARWVCRQIPQCYKVEVQESEGNIVIYEKDAVANEKTPAAGETE

SEQ ID NO: 20 - protein, 290 aa, Thermosynechococcus elongatus
MNCIIHRRAEFAASHRYWLPEWSEAENLARFGANSRFPGHGHNYELFVSMEGVVDDFGMVLNLSDVKHIIRREVIEPLNFSYLNEVWPEFQATLPTTEHIARVIWDRLFPHLPLVRIRLFEHPRLWADYTGDPMEAYLSVGAHFSAAHRLALEDLSYEENCRIYGKCARPHGHGHNYHVEITVKGSIHPRTGMVVDLVKLEEVLKEQVIEPLDHTFLNKDIPYFATVVPTAENIAIYIAHLLQEPVRQLGATLHRVKLIESPNNSCEILCEELPPRNEVISGALPVLERV

SEQ ID NO: 21 - protein, 147 aa, Streptococcus thermophilus
MFFAPKEIKTETGESLVYNLHRTMVSKEFTFDAAHHLFNYEGKCKSLHGHTYHLQIAVSGYLDDRGMTYDFGDLKNIYKNHLEPYLDHRYLNESLPYMNTTAENMVFWIFQTTSKYLSEERELRLEYVRLYETPTAFAEFRREWLDD

SEQ ID NO: 22 - protein, 291 aa, Acaryochloris marina
MKCLIHRRAEFSASHRYWLPELSKSENQEKFGQCTRSPGHGHNYELFVSMWGELDQYGMVLNLSNVKQVIKREVTAPLNFSYLNEVWPEFKETLPTTEHLARVIWQRLEPHLPIVNIQLFEHPKLWADYKGAGMEAYLTVGSHFSAAHRLALPELSFEENCEIYGKCARPHGHGHNYHLEVTVKGEVDARTGMIVDLVALQSLVDDVVLDPLDHTFLNKDIPYFEKVVPTAENIAFYIAKLLREPILKIGAELHRIKLIESPNNSCEVLCSDLFDTAPMLSGRMGEPALVG

SEQ ID NO: 23 - protein, 261 aa, Homo sapiens
MEGGLGRAVCLLTGASRGFGRTLAPLLASLLSPGSVLVLSARNDEALRQLEAELGAERSGLRVVRVPADLGAEAGLQQLLGALRELPRPKGLQRLLLINNAGSLGDVSKGFVDLSDSTQVNNYWALNLTSMLCLTSSVLKAFPDSPGLNRTVVNISSLCALQPFKGWALYCAGKAARDMLFQVLALEEPNVRVLNYAPGPLDTDMQQLARETSVDPDMRKGLQELKAKGKLVDCKVSAQKLLSLLEKDEFKSGAHVDFYDK

SEQ ID NO: 24 - protein, 262 aa, Rattus norvegicus
MEGGRLGCAVCVLTGASRGFGRALAPQLAGLLSPGSVLLLSARSDSMLRQLKEELCTQQPGLQVVLAAADLGTESGVQQLLSAVRELPRPERLQRLLLINNAGTLGDVSKGFLNINDLAEVNNYWALNLTSMLCLTTGTLNAFSNSPGLSKTVVNISSLCALQPFKGWGLYCAGKAARDMLYQVLAVEEPSVRVLSYAPGPLDTNMQQLARETSMDPELRSRLQKLNSEGELVDCGTSAQKLLSLLQRDTFQSGAHVDFYDI

SEQ ID NO: 25 - protein, 261 aa, Mus musculus
MEADGLGCAVCVLTGASRGFGRALAPQLARLLSPGSVMLVSARSESMLRQLKEELGAQQPDLKVVLAAADLGTEAGVQRLLSAVRELPRPEGLQRLLLINNAATLGDVSKGFLNVNDLAEVNNYWALNLTSMLCLTSGTLNAFQDSPGLSKTVVNISSLCALQPYKGWGLYCAGKAARDMLYQVLAAEEPSVRVLSYAPGPLDNDMQQLARETSKDPELRSKLQKLKSDGALVDCGTSAQKLLGLLQKDTFQSGAHVDFYD

SEQ ID NO: 26 - protein, 267 aa, Bos taurus
MEGSVGKVGGLGRTLCVLTGASRGFGRTLAQVLAPLMSPRSVLVLSARNDEALRQLETELGAEWPGLRIVRVPADLGAETGLQQLVGALCDLPRPEGLQRVLLINNAGTLGDVSKRWVDLTDPTEVNNYWTLNLTSTLCLTSSILQAFPDSPGLSRTVVNISSICALQPFKGWGLYCAGKAARNMMFQVLAAEEPSVRVLSYGPGPLDTDMQQLARETSVDPDLRKSLQELKRKGELVDCKISAQKLLSLLQNDKFESGAHIDFYDE

SEQ ID NO: 27 - protein, 261 aa, Danio rerio
MSTASGFGKALVIITGASRGFGRALALSVAARVSPGSVLVLAARSEEQLLELKSALTRGETGLTVRCVPVDLGCEAGVEKLIAETRDIQPDIQHLLLFHNAASLGDVSRYCRDFTNMEELNSYLSLNVSSALCLTAGVLRTYPKRSGLTRVIVNISSLCALRPFPTWVQYCSGKAARDMMFRVLAEEEPELRVLNYAPGPLDTDMQREARSSCADSKLRNTFSQMHANGQLLTCDQSIQKLMSVLLEDKYSSGEHLDYYDL

SEQ ID NO: 28 - protein, 263 aa, Xenopus laevis
MTAARAGALGSVLCVLTGASRGFGRTLAHELCPRVLPGSTLLLVSRTEEALKGLAEELGHEFPGVRVRWAAADLSTTEGVSATVRAARELQAGTAHRLLIINNAGSIGDVSKMFVDFSAPEEVTEYMKFNVSSPLCLTASLLKTFPRRPDLQRLVVNVSSLAALQPYKSWVLYCSGKAARDMMFRVLAEEEDDVRVLSYAPGPLDTDMHEVACTQTADPELRRAIMDRKEKGNMVDIRVSANKMLDLLEADAYKSGDHIDFYD

SEQ ID NO: 29 - protein, 118 aa, Pseudomonas aeruginosa
MTALTQAHCEACRADAPHVSDEELPVLLRQIPDWNIEVRDGIMQLEKVYLFKNFKHALAFTNAVGEISEAEGHHPGLLTEWGKVTVTWWSHSIKGLHRNDFIMAARTDEVAKTAEGRK

SEQ ID NO: 30 - protein, 104 aa, Bacillus cereus var. anthracis
MMLRLTEEEVQEELLKLDKWVVKDEKWIERKYMFSDYLKGVEFVSEAAKLSEEHNHHPFILIQYKAVIITLSSWNAKGLTKLDFELAKQFDELFVQNEKAVIRK

SEQ ID NO: 31 - protein, 188 aa, Corynebacterium genitalium
MSDTLDALDIHEPDEAFLMATEAEVEVPSQPCALAVLVSDHKQGGAIDEGTDRLVFELLQEIGFKVDGVVYVKSKKSEIRKVIETAVVGGVDLVVTVGGTGVGPRDKAPEATRGVIDQLVPGVAQAVRASGQACGAVDACTSRGICGVSGSTVVVNLAPSRAAIRDGISTISPLVAHLISELRKYSVQ

SEQ ID NO: 32 - protein, 63 aa, Lactobacillus ruminis
MVKLFPSENARRWHRWNHEVLLLVNIQCSLKQPLWSAEGKVDKNREKCAAFVYRLVEIQDARI

SEQ ID NO: 33 - protein, 96 aa, Rhodobacteraceae bacterium
MSERLFDDTRGPLLDPLFATGWAMVEGRDAIEKHYKFKNFADAFGWMTRAAIWSEKWDHHPEWLNVYNKVHVVLTTHSVDGLSPLDVKLARKFDSL

SEQ ID NO: 34 - protein, 104 aa, Homo sapiens
MAGKAHRLSAEERDQLLPNLRAVGWNELEGRDAIFKQFHFKDFNRAFGFMTRVALQAEKLDHHPEWFNVYNKVHITLSTHECAGLSERDINLASFIEQVAVSMT

SEQ ID NO: 35 - protein, 244 aa, Homo sapiens
MAAAAAAGEARRVLVYGGRGALGSRCVQAFRARNWWVASVDVVENEEASASIIVKMTDSFTEQADQVTAEVGKLLGEEKVDAILCVAGGWAGGNAKSKSLFKNCDLMWKQSIWTSTISSHLATKHLKEGGLLTLAGAKAALDGTPGMIGYGMAKGAVHQLCQSLAGKNSGMPPGAAAIAVLPVTLDTPMNRKSMPEADFSSWTPLEFLVETFHDWITGKNRPSSGSLIQVVTTEGRTELTPAYF

SEQ ID NO: 36 - protein, 241 aa, Rattus norvegicus
MAASGEARRVLVYGGRGALGSRCVQAFRARNWWVASIDVVENEEASASVIVKMTDSFTEQADQVTAEVGKLLGDQKVDAILCVAGGWAGGNAKSKSLFKNCDLMWKQSIWTSTISSHLATKHLKEGGLLTLAGAKAALDGTPGMIGYGMAKGAVHQLCQSLAGKNSGMPSGAAAIAVLPVTLDTPMNRKSMPEADFSSWTPLEFLVETFHDWITGNKRPNSGSLIQVVTTDGKTELTPAYF

SEQ ID NO: 37 - protein, 243 aa, Sus scrofa
MAAAAAGEARRVLVYGGRGALGSRCVQAFRARNWWVASIDVVENEEASANVVVKMTDSFTEQADQVTAEVGKLLGTEKVDAILCVAGGWAGGNAKSKSLFKNCDLMWKQSMWTSTISSHLATKHLKEGGLLTLAGAKAALDGTPGMIGYGMAKGAVHQLCQSLAGKDSGMPSGAAAIAVLPVTLDTPLNRKSMPHADFSSWTPLEFLVETFHDWIIEKNRPSSGSLIQVVTTQGKTELTPAYF

SEQ ID NO: 38 - protein, 242 aa, Bos taurus
MAAAAGEARRVLVYGGRGALGSRCVQAFRARNWWVASIDVQENEEASANVVVKMTDSFTEQADQVTAEVGKLLGTEKVDAILCVAGGWAGGNAKSKSLFKNCDLMWKQSVWTSTISSHLATKHLKEGGLLTLAGARAALDGTPGMIGYGMAKAAVHQLCQSLAGKSSGLPPGAAAVALLPVTLDTPVNRKSMPEADFSSWTPLEFLVETFHDWITEKNRPSSGSLIQVVTTEGKTELTAASP

SEQ ID NO: 39 - protein, 217 aa, Escherichia coli
MDIISVALKRHSTKAFDASKKLTPEQAEQIKTLLQYSPSSTNSQPWHFIVASTEEGKARVAKSAAGNYVFNERKMLDASHVVVFCAKTAMDDVWLKLVVDQEDADGRFATPEAKAANDKGRKFFADMHRKDLHDDAEWMAKQVYLNVGNFLLGVAALGLDAVPIEGFDAAILDAEFGLKEKGYTSLVVVPVGHHSVEDFNATLPKSRLPQNITLTEV

SEQ ID NO: 40 - protein, 231 aa, Dictyostelium discoideum
MSKNILVLGGSGALGAEVVKFFKSKSWNTISIDFRENPNADHSFTIKDSGEEEIKSVIEKINSKSIKVDTFVCAAGGWSGGNASSDEFLKSVKGMIDMNLYSAFASAHIGAKLLNQGGLFVLTGASAALNRTSGMIAYGATKAATHHIIKDLASENGGLPAGSTSLGILPVTLDTPTNRKYMSDANFDDWTPLSEVAEKLFEWSTNSDSRPTNGSLVKFETKSKVTTWTNL

SEQ ID NO: 41 - protein, 430 aa, Acidobacterium capsulatum
SAGQAYADYLHLVQPYTNGNRHPRAWGWVRGNGTPIGAMAEMLAAAINPHLGGGDQSPTYVEERCLQWLAQVMGMPATATGILTSGGTMANLLGLAVARHAKAGFDVRAEGLAAHTPLTVYASSEAHMWAGNAMDLLGLGSSRLRSIPVDENFRIDLAALRLKIREDRAAGLQPIAVIGNAGTVNTGAVDDLEALAALCREEELWFHVDGAFGALLKLSPRHASLVRGLEQADSLAFDLHKWMYLPFEIGCVLVANGEEHRAAFASSASYLEGAKRGILATGLIFADRGLELTRGFKALKLWMALKAHGLNAFSEMIEQNMAQARYLERRVLEEPELELLAPRSMNIVCFRYRGRGAAGDELLNALNRELVLRLQESGEFVVSGTMLKGRYALRIANTNHRSRLQDFEDLVQWSLKLGCEIEAESQAART

SEQ ID NO: 42 - protein, 480 aa, Rattus norvegicus
MDSREFRRRGKEMVDYIADYLDGIEGRPVYPDVEPGYLRALIPTTAPQEPETYEDIIRDIEKIIMPGVTHWHSPYFFAYFPTASSYPAMLADMLCGAIGCIGFSWAASPACTELETVMMDWLGKMLELPEAFLAGRAGEGGGVIQGSASEATLVALLAARTKMIRQLQAASPELTQAALMEKLVAYTSDQAHSSVERAGLIGGVKIKAIPSDGNYSMRAAALREALERDKAAGLIPFFVVVTLGTTSCCSFDNLLEVGPICNQEGVWLHIDAAYAGSAFICPEFRYLLNGVEFADSFNFNPHKWLLVNFDCSAMWVKKRTDLTEAFNMDPVYLRHSHQDSGLITDYRHWQIPLGRRFRSLKMWFVFRMYGVKGLQAYIRKHVKLSHEFESLVRQDPRFEICTEVILGLVCFRLKGSNQLNETLLQRINSAKKIHLVPCRLRDKFVLRFAVCSRTVESAHVQLAWEHIRDLASSVLRAEKE

SEQ ID NO: 43 - protein, 486 aa, Sus scrofa
MNASDFRRRGKEMVDYMADYLEGIEGRQVYPDVQPGYLRPLIPATAPQEPDTFEDILQDVEKIIMPGVTHWHSPYFFAYFPTASSYPAMLADMLCGAIGCIGFSWAASPACTELETVMMDWLGKMLQLPEAFLAGEAGEGGGVIQGSASEATLVALLAARTKVVRRLQAASPGLTQGAVLEKLVAYASDQAHSSVERAGLIGGVKLKAIPSDGKFAMRASALQEALERDKAAGLIPFFVVATLGTTSCCSFDNLLEVGPICHEEDIWLHVDAAYAGSAFICPEFRHLLNGVEFADSFNFNPHKWLLVNFDCSAMWVKRRTDLTGAFKLDPVYLKHSHQGSGLITDYRHWQLPLGRRFRSLKMWFVFRMYGVKGLQAYIRKHVQLSHEFEAFVLQDPRFEVCAEVTLGLVCFRLKGSDGLNEALLERINSARKIHLVPCRLRGQFVLRFAICSRKVESGHVRLAWEHIRGLAAELLAAEEGKAEIKS

SEQ ID NO: 44 - protein, 480 aa, Homo sapiens
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 45 - protein, 503 aa, Capsicum annuum
MGSLDSNNSTQTQSNVTKFNPLDPEEFRTQAHQMVDFIADYYKNIESYPVLSQVEPGYLRNHLPENAPYLPESLDTIMKDVEKHIIPGMTHWLSPNFFAFFPATVSSAAFLGEMLCNCFNSVGFNWLASPAMTELEMIIMDWLANMLKLPECFMFSGTGGGVIQGTTSEAILCTLIAARDRKLENIGVDNIGKLVVYGSDQTHSMYAKACKAAGIFPCNIRAISTCVENDFSLSPAVLRGIVEVDVAAGLVPLFLCATVGTTSTTAIDPISELGELANEFDIWLHVDAAYGGSACICPEFRQYLDGIERANSFSLSPHKWLLSYLDCCCMWVKEPSVLVKALSTNPEYLRNKRSEHGSVVDYKDWQIGTGRKFKSLRLWLIMRSYGVANLQSHIRSDVRMAKMFEGLVRSDPYFEVIVPRRFSLVCFRFNPDKEYEPAYTELLNKRLLDNVNSTGRVYMTHTVAGGIYMLRFAVGATFTEDRHLICAWKLIKDCADALLRNCQ

SEQ ID NO: 46 - protein, 281 aa, Drosophila caribiana
  misc_feature (79)..(79): Xaa can be any naturally occurring amino acid
MLDLPAEFLACSGGKGGGVIQGTASESTLVALLGAKAKKLQEVKAEHPEWDDHTIIGKLVGYTSAQSHSSVERAGLLGXIKLRSVPADEHNRLRGDALEKAIEKDLAEGLIPFYAVVTLGTTNSCAFDRLDECGPVANKHKVWVHVDAAYAGSAFICPEYRHHMKGIETADSFNFNPHKWMLVNFDCSAMWLKDPSWVVNAFNVDPLYLKHDMQGSAPDYRHWQIPLGRRFRALKLWFVLRLYGVENLQAHIRRHCGFAQQFADLCVADERFELAAEVNMG

SEQ ID NO: 47 - protein, 478 aa, Maricaulis maris
MHGRCKKLRLPPGMIMKLEEFGLWSRRIADWSKTYLETLRERPVRPATRPADVLNALPVTPPEDATDMAEIFADFERIVPDAMTHWQHPRFFAYFPANAAPASILAEQLVSTMAAQCMLWQTSPAATEMETRMVDWLRQALGLPDGWRGVIQDSASSATLSAVMTMRERALDWRGIRSGLAGEKAPRIYASAQTHSSVDKACWVAGIGQDNLVKIATTDDYGMDPDALRAAIRADRAAGHLPAGIVICVGGTAIGASDPVAAIIEVARAEGLYTHIDAAWAGSAMICPELRHIWEGAEGADSIVFNPHKWLGAQFDCSVQFLRDPTDQLKSLTLRPDYLETPGMDDAVNYSEWTIPLGRRFRALKLWFLIRAYGLEGLRTRIRNHIAWSNEACEAIRDLPGLEIVTEPRFSLFSFACTAGDEATADLLERINSDGRTYLTQTRHEGRYVIRLQVGQFDCTRADVMEAVAVIGELRGEG

SEQ ID NO: 48 - protein, 523 aa, Oryza sativa
MGSLDANPAAAYAAFAADVEPFRPLDADDVRSYLHKAVDFVYDYYKSVESLPVLPGVEPGYLLRLLQSAPPSSSAPFDIAMKELREAVVPGMTHWASPNFFAFFPATNSAAAIAGELIASAMNTVGFTWQAAPAATELEVLALDWLAQLLGLPASFMNRTVAGGRGTGGGVILGTTSEAMLVTLVAARDAALRRSGSNGVAGITRLTVYAADQTHSTFFKACRLAGFDPANIRSIPTGAETDYGLDPARLLEAMQADADAGLVPTYVCATVGTTSSNAVDPVGAVADVAARFAAWVHVDAAYAGSACICPEFRHHLDGVERVDSISMSPHKWLMTCLDCTCLYVRDTHRLTGSLETNPEYLKNHASDSGEVTDLKDMQVGVGRRFRGLKLWMVMRTYGAGKLQEHIRSDVAMAKTFEDLVRGDDRFEVVVPRNFALVCFRIRPRKSGAAIAAGEAEAEKANRELMERLNKTGKAYVAHTVVGGRFVLRFAVGSSLQEERHVRSAWELIKKTTTEIVADAGEDK

SEQ ID NO: 49 - protein, 470 aa, Pseudomonas putida
MTPEQFRQYGHQLIDLIADYRQTVGERPVMAQVEPGYLKAALPAQAPRQGEPFAAILDDVNQLVMPGLSHWQHPDFYGYFPSNGTLSSVLGDFLSTGLGVLGLSWQSSPALSELEETTLDWLRQLLGLSGQWSGVIQDTASTSTLVALICARERASDYALVRGGLQAQAKPLIVYVSAHAHSSVDKAALLAGFGRDNIRLIPTDERYALRPEALQVAIEQDLAAGNQPCAVVATTGTTATTALDPLRPIGEIAQAHGLWLHVDSAMAGSAMILPECRWMWDGIELADSLVVNAHKWLGVAFDCSIYYVRDPQHLIRVMSTNPSYLQSSVDGEVKNLRDWGIPLGRRFRALKLWFMLRSEGVEALQARLRRDLDNAQWLAGQIGAAAEWEVLAPVQLQTLCIRHRPAGLEGEALDAHTKGWAERLNASGDAYVTPATLDGRWMVRVSIGALPTEREHVEQLWARLQEVVKG

SEQ ID NO: 50 - protein, 500 aa, Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 51 - protein, 191 aa, Chlamydomonas reinhardtii
MAEESLDASVQPLGSTVFFGPVQPEMLDRIHELEAASYPEDEAATYEKLKFRIENASNVFLVALSAEGDGEPKVVGFVCGTQTRASKLTHESMSTHDADGALLCIHSVVVDAALRRRGLATRMLRAYTAFVAATSPGLTGIRLLTKQNLIPLYEGAGFTLLGPSDVEHGADLWYECAMELEAEEEAEAAEA

SEQ ID NO: 52 - protein, 207 aa, Bos taurus
MSTPSIHCLKPSPLHLPSGIPGSPGRQRRHTLPANEFRCLTPEDAAGVFEIEREAFISVSGNCPLNLDEVRHFLTLCPELSLGWFVEGRLVAFIIGSLWDEERLTQESLTLHRPGGRTAHLHALAVHHSFRQQGKGSVLLWRYLQHAGGQPAVRRAVLMCEDALVPFYQRFGFHPAGPCAVVVGSLTFTEMHCSLRGHAALRRNSDR

SEQ ID NO: 53 - protein, 205 aa, Gallus gallus
MPVLGAVPFLKPTPLQGPRNSPGRQRRHTLPASEFRCLSPEDAVSVFEIEREAFISVSGDCPLHLDEIRHFLTLCPELSLGWFEEGRLVAFIIGSLWDQDRLSQAALTLHNPRGTAVHIHVLAVHRTFRQQGKGSILMWRYLQYLRCLPCARRAVLMCEDFLVPFYEKCGFVAVGPCQVTVGTLAFTEMQHEVRGHAFMRRNSGC

SEQ ID NO: 54 - protein, 207 aa, Homo sapiens
MSTQSTHPLKPEAPRLPPGIPESPSCQRRHTLPASEFRCLTPEDAVSAFEIEREAFISVLGVCPLYLDEIRHFLTLCPELSLGWFEEGCLVAFIIGSLWDKERLMQESLTLHRSGGHIAHLHVLAVHRAFRQQGRGPILLWRYLHHLGSQPAVRRAALMCEDALVPFYERFSFHAVGPCAITVGSLTFMELHCSLRGHPFLRRNSGC

SEQ ID NO: 55 - protein, 205 aa, Mus musculus
MLNINSLKPEALHLPLGTSEFLGCQRRHTLPASEFRCLTPEDATSAFEIEREAFISVSGTCPLYLDEIRHFLTLCPELSLGWFEEGCLVAFIIGSLWDKERLTQESLTLHRPGGRTAHLHVLAVHRTFRQQGKGSVLLWRYLHHLGSQPAVRRAVLMCEDALVPFYEKFGFQAVGPCAITVGSLTFTELQCSLRCHAFLRRNSGC

SEQ ID NO: 56 - protein, 207 aa, Oryctolagus cuniculus
MSTLSTQPLKPKALHPPPGSPESPGHQRRHTLPASEFRCLTPEDAAGVFEIEREAFMSVSGSCPLYLDEIRHFLTLCPELSLGWFQEGRLVAFIIGSLWDKERLTQESLTLHRPGGRVAHLHVLAVHRACRQQGKGSVLLWRYLQHLGGQRAVRRAVLMCEDALVPFYERLGFRAVGPCAVTVGSLAFTELQCSVRGHACLRRKSGC

SEQ ID NO: 57 - protein, 207 aa, Ovis aries
MSTPSVHCLKPSPLHLPSGIPGSPGRQRRHTLPANEFRCLTPEDAAGVFEIEREAFISVSGNCPLNLDEVQHFLTLCPELSLGWFVEGRLVAFIIGSLWDEERLTQESLALHRPRGHSAHLHALAVHRSFRQQGKGSVLLWRYLHHVGAQPAVRRAVLMCEDALVPFYQRFGFHPAGPCAIVVGSLTFTEMHCSLRGHAALRRNSDR

SEQ ID NO: 58 - protein, 364 aa, Oryza sativa
MAQNVQENEQVMSTEDLLQAQIELYHHCLAFIKSMALRAATDLRIPDAIHCNGGAATLTDLAAHVGLHPTKLSHLRRLMRVLTLSGIFTVHDGDGEATYTLTRVSRLLLSDGVERTHGLSQMVRVFVNPVAVASQFSLHEWFTVEKAAAVSLFEVAHGCTRWEMIANDSKDGSMFNAGMVEDSSVAMDIILRKSSNVFRGINSLVDVGGGYGAVAAAVVRAFPDIKCTVLDLPHIVAKAPSNNNIQFVGGDLFEFIPAADVVLLKCILHCWQHDDCVKIMRRCKEAISARDAGGKVILIEVVVGIGSNETVPKEMQLLFDVFMMYTDGIEREEHEWKKIFLEAGFSDYKIIPVLGVRSIIEVYP

SEQ ID NO: 59 - protein, 345 aa, Homo sapiens
MGSSEDQAYRLLNDYANGFMVSQVLFAACELGVFDLLAEAPGPLDVAAVAAGVRASAHGTELLLDICVSLKLLKVETRGGKAFYRNTELSSDYLTTVSPTSQCSMLKYMGRTSYRCWGHLADAVREGRNQYLETFGVPAEELFTAIYRSEGERLQFMQALQEVWSVNGRSVLTAFDLSVFPLMCDLGGGAGALAKECMSLYPGCKITVFDIPEVVWTAKQHFSFQEEEQIDFQEGDFFKDPLPEADLYILARVLHDWADGKCSHLLERIYHTCKPGGGILVIESLLDEDRRGPLLTQLYSLNMLVQTEGQERTPTHYHMLLSSAGFRDFQFKKTGAIYDAILARK

SEQ ID NO: 60 - protein, 345 aa, Bos taurus
MCSQEGEGYSLLKEYANGFMVSQVLFAACELGVFELLAEALEPLDSAAVSSHLGSSPQGTELLLNTCVSLKLLQADVRGGKAVYANTELASTYLVRGSPRSQRDMLLYAGRTAYVCWRHLAEAVREGRNQYLKAFGIPSEELFSAIYRSEDERLQFMQGLQDVWRLEGATVLAAFDLSPFPLICDLGGGSGALAKACVSLYPGCRAIVFDIPGVVQIAKRHFSASEDERISFHEGDFFKDALPEADLYILARVLHDWTDAKCSHLLQRVYRACRTGGGILVIESLLDTDGRGPLTTLLYSLNMLVQTEGRERTPAEYRALLGPAGFRDVRCRRTGGTYDAVLARK

SEQ ID NO: 61 - protein, 432 aa, Rattus norwegicus
MAPGREGELDRDFRVLMSLAHGFMVSQVLFAALDLGIFDLAAQGPVAAEAVAQTGGWSPRGTQLLMDACTRLGLLRGAGDGSYTNSALSSTFLVSGSPQSQRCMLLYLAGTTYGCWAHLAAGVREGRNQYSRAVGISAEDPFSAIYRSEPERLLFMRGLQETWSLCGGRVLTAFDLSRFRVICDLGGGSGALAQEAARLYPGSSVCVFDLPDVIAAARTHFLSPGARPSVRFVAGDFFRSRLPRADLFILARVLHDWADGACVELLGRLHRACRPGGALLLVEAVLAKGGAGPLRSLLLSLNMMLQAEGWERQASDYRNLATRAGFPRLQLRRPGGPYHAMLARRGPRPGIITGVGSNTTGTGSFVTGIRRDVPGARSDAAGTGSGTGNTGSGIMLQGETLESEVSAPQAGSDVGGAGNEPRSGTLKQGDWK

SEQ ID NO: 62 - protein, 346 aa, Gallus gallus
MDSTEDLDYPQIIFQYSNGFLVSKVMFTACELGVFDLLLQSGRPLSLDVIAARLGTSIMGMERLLDACVGLKLLAVELRREGAFYRNTEISNIYLTKSSPKSQYHIMMYYSNTVYLCWHYLTDAVREGRNQYERAFGISSKDLFGARYRSEEEMLKFLAGQNSIWSICGRDVLTAFDLSPFTQIYDLGGGGGALAQECVFLYPNCTVTIYDLPKVVQVAKERLVPPEERRIAFHEGDFFKDSIPEADLYILSKILHDWDDKKCRQLLAEVYKACRPGGGVLLVESLLSEDRSGPVETQLYSLNMLVQTEGKERTAVEYSELLGAAGFREVQVRRTGKLYDAVLGRK

SEQ ID NO: 63 - protein, 345 aa, Macaca mulatta
MGSSGDDGYRLLNEYTNGFMVSQVLFAACELGVFDLLAEAPGPLDVAAVAAGVEASSHGTELLLDTCVSLKLLKVETRAGKAFYQNTELSSAYLTRVSPTSQCNLLKYMGRTSYGCWGHLADAVREGKNQYLQTFGVPAEDLFKAIYRSEGERLQFMQALQEVWSVNGRSVLTAFDLSGFPLMCDLGGGPGALAKECLSLYPGCKVTVFDVPEVVRTAKQHFSFPEEEEIHLQEGDFFKDPLPEADLYILARILHDWADGKCSHLLERVYHTCKPGGGILVIESLLDEDRRGPLLTQLYSLNMLVQTEGQERTPTHYHMLLSSAGFRDFQFKKTGAIYDAILVRK

SEQ ID NO: 64 - protein, 384 aa, Eschericia coli
MAKHLFTSESVSEGHPDKIADQISDAVLDAILEQDPKARVACETYVKTGMVLVGGEITTSAWVDIEEITRNTVREIGYVHSDMGFDANSCAVLSAIGKQSPDINQGVDRADPLEQGAGDQGLMFGYATNETDVLMPAPITYAHRLVQRQAEVRKNGTLPWLRPDAKSQVTFQYDDGKIVGIDAVVLSTQHSEEIDQKSLQEAVMEEIIKPILPAEWLTSATKFFINPTGRFVIGGPMGDCGLTGRKIIVDTYGGMARHGGGAFSGKDPSKVDRSAAYAARYVAKNIVAAGLADRCEIQVSYAIGVAEPTSIMVETFGTEKVPSEQLTLLVREFFDLRPYGLIQMLDLLHPIYKETAAYGHFGREHFPWEKTDKAQLLRDAAGLK

SEQ ID NO: 65 - protein, 382 aa, Saccharomyces cerevisiae
MAGTFLFTSESVGEGHPDKICDQVSDAILDACLAEDPHSKVACETAAKTGMIMVFGEITTKAQLDYQKIVRDTIKKIGYDDSAKGFDYKTCNVLVAIEQQSPDIAQGVHEEKDLEDIGAGDQGIMFGYATDETPEGLPLTILLAHKLNMAMADARRDGSLAWLRPDTKTQVTVEYKDDHGRWVPQRIDTVVVSAQHADEITTEDLRAQLKSEIIEKVIPRDMLDENTKYFIQPSGRFVIGGPQGDAGLTGRKIIVDAYGGASSVGGGAFSGKDYSKVDRSAAYAARWVAKSLVAAGLCKRVQVQFSYAIGIAEPLSLHVDTYGTATKSDEEIIDIISKNFDLRPGVLVKELDLARPIYLPTASYGHFTNQEYPWEKPKTLKF

SEQ ID NO: 66 - protein, 384 aa, Saccharomyces cerevisiae
MSKSKTFLFTSESVGEGHPDKICDQVSDAILDACLEQDPFSKVACETAAKTGMIMVFGEITTKARLDYQQIVRDTIKKIGYDDSAKGFDYKTCNVLVAIEQQSPDIAQGLHYEKSLEDLGAGDQGIMFGYATDETPEGLPLTILLAHKLNMAMADARRDGSLPWLRPDTKTQVTVEYEDDNGRWVPKRIDTVVISAQHADEISTADLRTQLQKDIVEKVIPKDMLDENTKYFIQPSGRFVIGGPQGDAGLTGRKIIVDAYGGASSVGGGAFSGKDYSKVDRSAAYAARWVAKSLVAAGLCKRVQVQFSYAIGIAEPLSLHVDTYGTATKSDDEIIEIIKKNFDLRPGVLVKELDLARPIYLPTASYGHFTNQEYSWEKPKKLEF

SEQ ID NO: 67 - protein, 396 aa, Bos taurus
MNGPVDGLCDHSLSEEGAFMFTSESVGEGHPDKICDQISDAVLDAHLKQDPNAKVACETVCKTGMVLLCGEITSMAMVDYQRVVRETIQHIGYDDSAKGFDFKTCNVLVALEQQSPDIAQCVHLDRNEEDVGAGDQGLMFGYATDETEECMPLTIMLAHRLNARMAELRRSGQLPWLQPDSKTQVTVQYTQDNGAVIPMRVHTVVISVQHNEDITLEDMRRALKEQVIRAVVPARYLDEDTIYHLQPSGRFVIGGPQGDAGVTGRKIIVDTYGGWGAHGGGAFSGKDYTKVDRSAAYAARWVAKSLVKAGLCRRVLVQVSYAIGVAEPLSISIFTYGTSQKTERELLDVVNKNFDLRPGVIVRDLDLKKPIYQKTACYGHFGRSEFPWEVPKKLVF

SEQ ID NO: 68 - protein, 395 aa, Homo sapiens
MNGPVDGLCDHSLSEGVFMFTSESVGEGHPDKICDQISDAVLDAHLKQDPNAKVACETVCKTGMVLLCGEITSMAMVDYQRVVRDTIKHIGYDDSAKGFDFKTCNVLVALEQQSPDIAQCVHLDRNEEDVGAGDQGLMFGYATDETEECMPLTIILAHKLNARMADLRRSGLLPWLRPDSKTQVTVQYMQDNGAVIPVRIHTIVISVQHNEDITLEEMRRALKEQVIRAVVPAKYLDEDTVYHLQPSGRFVIGGPQGDAGVTGRKIIVDTYGGWGAHGGGAFSGKDYTKVDRSAAYAARWVAKSLVKAGLCRRVLVQVSYAIGVAEPLSISIFTYGTSQKTERELLDVVHKNFDLRPGVIVRDLDLKKPIYQKTACYGHFGRSEFPWEVPRKLVF

SEQ ID NO: 69 - protein, 400 aa, Bacillus subtilis
MSKNRRLFTSESVTEGHPDKICDQISDSILDEILKKDPNARVACETSVTTGLVLVSGEITTSTYVDIPKTVRQTIKEIGYTRAKYGFDAETCAVLTSIDEQSADIAMGVDQALEAREGTMSDEEIEAIGAGDQGLMFGYACNETKELMPLPISLAHKLARRLSEVRKEDILPYLRPDGKTQVTVEYDENNKPVRIDAIVISTQHHPEITLEQIQRNIKEHVINPVVPEELIDEETKYFINPTGRFVIGGPQGDAGLTGRKIIVDTYGGYARHGGGAFSGKDATKVDRSAAYAARYVAKNIVAAELADSCEVQLAYAIGVAQPVSISINTFGSGKASEEKLIEVVRNNFDLRPAGIIKMLDLRRPIYKQTAAYGHFGRHDVDLPWERTDKAEQLRKEALGE

SEQ ID NO: 70 - protein, 581 aa, Saccharomyces cerevisiae
MSKQFSHTTNDRRSSIIYSTSVGKAGLFTPADYIPQESEENLIEGEEQEGSEEEPSYTGNDDETEREGEYHSLLDANNSRTLQQEAWQQGYDSHDRKRLLDEERDLLIDNKLLSQHGNGGGDIESHGHGQAIGPDEEERPAEIANTWESAIESGQKISTTFKRETQVITMNALPLIFTFILQNSLSLASIFSVSHLGTKELGGVTLGSMTANITGLAAIQGLCTCLDTLCAQAYGAKNYHLVGVLVQRCAVITILAFLPMMYVWFVWSEKILALMIPERELCALAANYLRVTAFGVPGFILFECGKRFLQCQGIFHASTIVLFVCAPLNALMNYLLVWNDKIGIGYLGAPLSVVINYWLMTLGLLIYAMTTKHKERPLKCWNGIIPKEQAFKNWRKMINLAIPGVVMVEAEFLGFEVLTIFASHLGTDALGAQSIVATIASLAYQVPFSISVSTSTRVANFIGASLYDSCMITCRVSLLLSFVCSSMNMFVICRYKEQIASLFSTESAVVKMVVDTLPLLAFMQLFDAFNASTAGCLRGQGRQKNRWVHQPSRILLPRCAHGICVSIPVSSGCRRLMVGYN

SEQ ID NO: 71 - protein, 569 aa, Pichia stipitis
MSSLTSREATPLLAPINSAFSDIDESEIVAYIDQRRVSIASAGPDRIPPSFLTSKSPIPHERDRLTHSISRNSLYVPDISDIISNSDIAPEPETDLATEVKVLLYYSVPLVVTFLLQYSLTVASVFSVGRLGSTELAAVSLSSMTANISGYAIIQGVSTCLDTLCAQSFGRKDYNTVGLHFMRCNYMLLLLFVPIFFLWVFGAEPVLHSIIGKEEAKLCQLAARYLRILSFGMPGFILFENGKHFLQSQGIFHASTYVLVICAPLNALLNYLLVWDKTVGIGFAGAPLSVVITNWLMCVLLFAYIFYVKGYQCWPKQQLSDSVYFTSWTKMINLSVPGVLMVEAEWLAFEIITFTASKFGTEVLAAQSIVSTTCVLMYQIPFAISIAASTRIAWYIGAASIKSAKIATSATIRTSLSLGIVNCCIIYVFREFFASLYTNDKKVIKLASEVLIIGAVYQINDFLSCATGGVLRGQGRQKIGGILNLISYYLIALPVAFLCAFYFKLGLLGLWIGMIIALFFISVSQYYFVATSNWEAVINECINEGIMEEGNITIDAHSTIPSMSSSTYV

SEQ ID NO: 72 - protein, 449 aa, Sacharomyces cerevisiae
MSAPAQNYKIADISLAAFGRKEIELAEHEMPGLMAIRKAYGDVQPLKGARIAGCLHMTIQTAVLIETLVALGAEVTWSSCNIYSTQDHAAAAIAASGVPVFAWKGETEEEYLWCIEQQLFAFKDNKKLNLILDDGGDLTTLVHEKHPEMLEDCFGLSEETTTGVHHLYRMVKEGKLKVPAINVNDSVTKSKFDNLYGCRESLVDGIKRATDVMLAGKVAVVAGYGDVGKGCAAALRGMGARVLVTEIDPINALQAAMEGYQVVTMEDASHIGQVFVTTTGCRDIINGEHFINMPEDAIVCNIGHFDIEIDVAWLKANAKECINIKPQVDRYLLSSGRHVILLANGRLVNLGCATGHSSFVMSCSFSNQVLAQIALFKSNDKSFREKHIEFQKTGPFEVGVHVLPKILDEAVAKFHLGNLGVRLTKLSKVQSEYLGIPEEGPFKADHYRY

SEQ ID NO: 73 - protein, 432 aa, Homo sapiens
MSDKLPYKVADIGLAAWGRKALDIAENEMPGLMRMRERYSASKPLKGARIAGCLHMTVETAVLIETLVTLGAEVQWSSCNIFSTQDHAAAAIAKAGIPVYAWKGETDEEYLWCIEQTLYFKDGPLNMILDDGGDLTNLIHTKYPQLLPGIRGISEETTTGVHNLYKMMANGILKVPAINVNDSVTKSKFDNLYGCRESLIDGIKRATDVMIAGKVAVVAGYGDVGKGCAQALRGFGARVIITEIDPINALQAAMEGYEVTTMDEACQEGNIFVTTTGCIDIILGRHFEQMKDDAIVCNIGHFDVEIDVKWLNENAVEKVNIKPQVDRYRLKNGRRIILLAEGRLVNLGCAMGHPSFVMSNSFTNQVMAQIELWTHPDKYPVGVHFLPKKLDEAVAEAHLGKLNVKLTKLTEKQAQYLGMSCDGPFKPDHYRY

SEQ ID NO: 74 - protein, 432 aa, Bos taurus
MSDKLPYKVADISLAAWGRKALDLAENEMPGLMHMREMYSASKPLKGARIAGCLHMTVETAVLIETLVALGAEVRWSSCNIFSTQDHAAAAIAKAGIPVYAWKGETDEEYLWCIEQTLYFKDGPLNMILDDGGDLTNLIHTKYPQLLSGIRGISEETTTGVHNLYKMMAKGILKVPAINVNDSVTKSKFDNLYGCRESLIDGIKRATDVMIAGKVAVVAGYGDVGKGCAQALRGFGARVIITEIDPINALQAAMEGYEVTTMDEACQEGNIFVTTTGCTDIILGQHFEQMKDDAIVCNIGHFDVEIDVKWLNENAVEKVNIKPQVDRYLLKNGRRIILLAEGRLVNLGCAMGHPSFVMSNSFTNQVLAQIELWTHPDKYPVGVHFLPKKLDEAVAEAHLGKLNVKLTKLTEKQAQYLGVSREGPFKPDHYRY

SEQ ID NO: 75 - protein, 1227 aa, Escherichia coli
MSSKVEQLRAQLNERILVLDGGMGTMIQSYRLNEADFRGERFADWPCDLKGNNDLLVLSKPEVIAAIHNAYFEAGADIIETNTFNSTTIAMADYQMESLSAEINFAAAKLARACADEWTARTPEKPRYVAGVLGPTNRTASISPDVNDPAFRNITFDGLVAAYRESTKALVEGGADLILIETVFDTLNAKAAVFAVKTEFEALGVELPIMISGTITDASGRTLSGQTTEAFYNSLRHAEALTFGLNCALGPDELRQYVQELSRIAECYVTAHPNAGLPNAFGEYDLDADTMAKQIREWAQAGFLNIVGGCCGTTPQHIAAMSRAVEGLAPRKLPEIPVACRLSGLEPLNIGEDSLFVNVGERTNVTGSAKFKRLIKEEKYSEALDVARQQVENGAQIIDINMDEGMLDAEAAMVRFLNLIAGEPDIARVPIMIDSSKWDVIEKGLKCIQGKGIVNSISMKEGVDAFIHHAKLLRRYGAAVVVMAFDEQGQADTRARKIEICRRAYKILTEEVGFPPEDIIFDPNIFAVATGIEEHNNYAQDFIGACEDIKRELPHALISGGVSNVSFSFRGNDPVREAIHAVFLYYAIRNGMDMGIVNAGQLAIYDDLPAELRDAVEDVILNRRDDGTERLLELAEKYRGSKTDDTANAQQAEWRSWEVNKRLEYSLVKGITEFIEQDTEEARQQATRPIEVIEGPLMDGMNVVGDLFGEGKMFLPQVVKSARVMKQAVAYLEPFIEASKEQGKTNGKMVIATVKGDVHDIGKNIVGVVLQCNNYEIVDLGVMVPAEKILRTAKEVNADLIGLSGLITPSLDEMVNVAKEMERQGFTIPLLIGGATTSKAHTAVKIEQNYSGPTVYVQNASRTVGVVAALLSDTQRDDFVARTRKEYETVRIQHGRKKPRTPPVTLEAARDNDFAFDWQAYTPPVAHRLGVQEVEASIETLRNYIDWTPFFMTWSLAGKYPRILEDEVVGVEAQRLFKDANDMLDKLSAEKTLNPRGVVGLFPANRVGDDIEIYRDETRTHVINVSHHLRQQTEKTGFANYCLADFVAPKLSGKADYIGAFAVTGGLEEDALADAFEAQHDDYNKIMVKALADRLAEAFAEYLHERVRKVYWGYAPNENLSNEELIRENYQGIRPAPGYPACPEHTEKATIWELLEVEKHTGMKLTESFAMWPGASVSGWYFSHPDSKYYAVAQIQRDQVEDYARRKGMSVTEVERWLAPNLGYDAD

SEQ ID NO: 76 - protein, 767 aa, Saccharomyces cerevisiae
MVQSAVLGFPRIGPNRELKKATEGYWNGKITVDELFKVGKDLRTQNWKLQKEAGVDIIPSNDFSFYDQVLDLSLLFNVIPDRYTKYDLSPIDTLFAMGRGLQRKATETEKAVDVTALEMVKWFDSNYHYVRPTFSKTTQFKLNGQKPVDEFLEAKELGIHTRPVLLGPVSYLFLGKADKDSLDLEPLSLLEQLLPLYTEILSKLASAGATEVQIDEPVLVLDLPANAQAAIKKAYTYFGEQSNLPKITLATYFGTVVPNLDAIKGLPVAALHVDFVRAPEQFDEVVAAIGNKQTLSVGIVDGRNIWKNDFKKSSAIVNKAIEKLGADRVVVATSSSLLHTPVDLNNETKLDAEIKGFFSFATQKLDEVVVITKNVSGQDVAAALEANAKSVESRGKSKFIHDAAVKARVASIDEKMSTRAAPFEQRLPEQQKVFNLPLFPTTTIGSFPQTKDIRINRNKFNKGTISAEEYEKFINSEIEKVIRFQEEIGLDVLVHGEPERNDMVQYFGEQINGYAFTVNGWVQSYGSRYVRPPIIVGDLSRPKAMSVKESVYAQSITSKPVKGMLTGPITCLRWSFPRDDVDQKTQAMQLALALRDEVNDLEAAGIKVIQVDEPALREGLPLREGTERSAYYTWAAEAFRVATSGVANKTQIHSHFCYSDLDPNHIKALDADVVSIEFSKKDDANYIAEFKNYPNHIGLGLFDIHSPRIPSKDEFIAKISTILKSYPAEKFWVNPDCGLKTRGWEETRLSLTHMVEAAKYFREQYKN

SEQ ID NO: 77 - protein, 1265 aa, Homo sapiens
MSPALQDLSQPEGLKKTLRDEINAILQKRIMVLDGGMGTMIQREKLNEEHFRGQEFKDHARPLKGNNDILSITQPDVIYQIHKEYLLAGADIIETNTFSSTSIAQADYGLEHLAYRMNMCSAGVARKAAEEVTLQTGIKRFVAGALGPTNKTLSVSPSVERPDYRNITFDELVEAYQEQAKGLLDGGVDILLIETIFDTANAKAALFALQNLFEEKYAPRPIFISGTIVDKSGRTLSGQTGEGFVISVSHGEPLCIGLNCALGAAEMRPFIEIIGKCTTAYVLCYPNAGLPNTFGDYDETPSMMAKHLKDFAMDGLVNIVGGCCGSTPDHIREIAEAVKNCKPRVPPATAFEGHMLLSGLEPFRIGPYTNFVNIGERCNVAGSRKFAKLIMAGNYEEALCVAKVQVEMGAQVLDVNMDDGMLDGPSAMTRFCNLIASEPDIAKVPLCIDSSNFAVIEAGLKCCQGKCIVNSISLKEGEDDFLEKARKIKKYGAAMVVMAFDEEGQATETDTKIRVCTRAYHLLVKKLGFNPNDIIFDPNILTIGTGMEEHNLYAINFIHATKVIKETLPGARISGGLSNLSFSFRGMEAIREAMHGVFLYHAIKSGMDMGIVNAGNLPVYDDIHKELLQLCEDLIWNKDPEATEKLLRYAQTQGTGGKKVIQTDEWRNGPVEERLEYALVKGIEKHIIEDTEEARLNQKKYPRPLNIIEGPLMNGMKIVGDLFGAGKMFLPQVIKSARVMKKAVGHLIPFMEKEREETRVLNGTVEEEDPYQGTIVLATVKGDVHDIGKNIVGVVLGCNNFRVIDLGVMTPCDKILKAALDHKADIIGLSGLITPSLDEMIFVAKEMERLAIRIPLLIGGATTSKTHTAVKIAPRYSAPVIHVLDASKSVVVCSQLLDENLKDEYFEEIMEEYEDIRQDHYESLKERRYLPLSQARKSGFQMDWLSEPHPVKPTFIGTQVFEDYDLQKLVDYIDWKPFFDVWQLRGKYPNRGFPKIFNDKTVGGEARKVYDDAHNMLNTLISQKKLRARGVVGFWPAQSIQDDIHLYAEAAVPQAAEPIATFYGLRQQAEKDSASTEPYYCLSDFIAPLHSGIRDYLGLFAVACFGVEELSKAYEDDGDDYSSIMVKALGDRLAEAFAEELHERVRRELWAYCGSEQLDVADLRRLRYKGIRPAPGYPSQPDHTEKLTMWRLADIEQSTGIRLTESLAMAPASAVSGLYFSNLKSKYFAVGKISKDQVEDYALRKNISVAEVEKWLGPILGYDTD

SEQ ID NO: 78 - protein, 1265 aa, Bos taurus
MAPTLQDLTPSAGMKKTLQDEIEAILQERIMVLDGGMGTMIQRHKLSEEDFRGQEFKDHARPLKGNNDILSITQPNVIYQIHKEYLLAGADIIETNTFSSTSIAQADYGLEHLAYRMNMCSAGVARKAAEDISLQTGIKRYVAGALGPTNKTLSVSPSVERPDYRNITFDELVEAYKEQAKGLLDGGVDILLIETIFDTANAKAALFAVQKLFEEEYVPRPVFISGTIVDKSGRTLSGQTGEAFVISVSHADPLCIGLNCALGAAEMRPFIETIGKCTTAYVLCYPNAGLPNTFGDYDETPHVMAMHLKDFAVDGLVNIVGGCCGTTPDHIREIAEAVKNCKPRVPPATVFEGHMLLSGLEPFRIGPYTNFVNIGERCNVAGSRRFAKLIMAGNYEEALSVAKMQVEMGAQVLDINMDDGMLDGPSAMTRFCNFIASEPDIAKVPLCIDSSNFAVIEAGLKCCQGKCIVNSISLKEGEDDFLEKARKIKKFGAAVVVMAFDEEGQATETDPKIRVCTRAYHLLLKKLGFNPNDIIFDPNILTIGTGMEEHNLYAVNFINATKVIKETLPGAKVSGGLSNLSFSFRGMEAIREAMHGVFLYHAIKFGMDMGIVNAGSLPVYDDIHKELLQLCEDLIWNRDPEATEKLLHYAQTQGKGGKKVIQTDEWRNGPLEERLEYALVKGIEKYIIEDTEEARLNQEKYPRPLNIIEGPLMNGMKIVGDLFGAGKMFLPQVIKSARVMKKAVGHLIPFMEKEREETKVLTGKIEDEDPYQGTIVLATVKGDVHDIGKNIVGVVLGCNNFRVIDLGVMTPCDKILKAALDHKADIIGLSGLITPSLDEMIFVAKEMERLAIKIPLLIGGATTSRTHTAVKIAPRYSAPVIHVLDASKSVVVCSQLLDENLKDEYFEEILEEYEDIRQDHYESLKERRYLTLRQARENGFHIDWLSEPPPVKPTFLGTRVFEDYDLQKLVDYIDWKPFFDVWQLRGKYPNRGFPKIFDDKTVGEEAKKVYDDAQNMLQALISQKKLQARGVVGFWPAQSIQDDIHLYAEGAVPQASEPIATFYGLRQQAEKDSASSDPYLCLSDFIAPLHSGIPDYLGLFAVACFGVEELSKAYEEECDDYSSIMVKALGDRLAEAFAEELHERARRELWGYCSGEQLAVADLRRLRYEGIRPAPGYPSQPDHTEKLTVWRLADVEQRTGIRLTESLAMAPASAVSGLYFSNLKSKYFAVGKISKDQIEDYASRKNMSVAEVEKWLGPILGYDTD

SEQ ID NO: 79 - protein, 652 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 80 - protein, 572 aa, Bacillus subtilis
MNLKALPAIEGDHNLKNYEETYRHFDWAEAEKHFSWHETGKLNAAYEAIDRHAESFRKNKVALYYKDAKRDEKYTFKEMKEESNRAGNVLRRYGNVEKGDRVFIFMPRSPELYFIMLGAIKIGAIAGPLFEAFMEGAVKDRLENSEAKVVVTTPELLERIPVDKLPHLQHVFVVGGEAESGTNIINYDEAAKQESTRLDIEWMDKKDGFLLHYTSGSTGTPKGVLHVHEAMIQQYQTGKWVLDLKEEDIYWCTADPGWVTGTVYGIFAPWLNGATNVIVGGRFSPESWYGTIEQLGVNVWYSAPTAFRMLMGAGDEMAAKYDLTSLRHVLSVGEPLNPEVIRWGHKVFNKRIHDTWWMTETGSQLICNYPCMDIKPGSMGKPIPGVEAAIVDNQGNELPPYRMGNLAIKKGWPSMMHTIWNNPEKYESYFMPGGWYVSGDSAYMDEEGYFWFQGRVDDVIMTSGERVGPFEVESKLVEHPAIAEAGVIGKPDPVRGEIIKAFIALREGFEPSDKLKEEIRLFVKQGLAAHAAPREIEFKDKLPKTRSGKIMRRVLKAWELNLPAGDLSTMED

SEQ ID NO: 81 - protein, 652 aa, Salmonella enterica
MSQTHKHAIPANIADRCLINPEQYETKYKQSINDPDTFWGEQGKILDWITPYQKVKNTSFAPGNVSIKWYEDGTLNLAANCLDRHLQENGDRTAIIWEGDDTSQSKHISYRELHRDVCRFANTLLDLGIKKGDVVAIYMPMVPEAAVAMLACARIGAVHSVIFGGFSPEAVAGRIIDSSSRLVITADEGVRAGRSIPLKKNVDDALKNPNVTSVEHVIVLKRTGSDIDWQEGRDLWWRDLIEKASPEHQPEAMNAEDPLFILYTSGSTGKPKGVLHTTGGYLVYAATTFKYVFDYHPGDIYWCTADVGWVTGHSYLLYGPLACGATTLMFEGVPNWPTPARMCQVVDKHQVNILYTAPTAIRALMAEGDKAIEGTDRSSLRILGSVGEPINPEAWEWYWKKIGKEKCPVVDTWWQTETGGFMITPLPGAIELKAGSATRPFFGVQPALVDNEGHPQEGATEGNLVITDSWPGQARTLFGDHERFEQTYFSTFKNMYFSGDGARRDEDGYYWITGRVDDVLNVSGHRLGTAEIESALVAHPKIAEAAVVGIPHAIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 82 - protein, 316 aa, Escherichia coli
MSKRKVAIIGSGNIGTDLMIKILRHGQHLEMAVMVGIDPQSDGLARARRMGVATTHEGVIGLMNMPEFADIDIVFDATSAGAHVKNDAALREAKPDIRLIDLTPAAIGPYCVPVVNLEANVDQLNVNMVTCGGQATIPMVAAVSRVARVHYAEIIASIASKSAGPGTRANIDEFTETTSRAIEVVGGAAKGKAIIVLNPAEPPLMMRDTVYVLSDEASQDDIEASINEMAEAVQAYVPGYRLKQRVQFEVIPQDKPVNLPGVGQFSGLKTAVWLEVEGAAHYLPAYAGNLDIMTSSALATAEKMAQSLARKAGEAA

SEQ ID NO: 83 - protein, 302 aa, Bacillus sp.
MNRTVKVAILGSGNIGTDLMYKILKKRWVLELSMIAGIDPQSEGLARARAEGVYATAGGIDAILEDPEIKIVFDATSAKAHLKHAKRLKEAGKVAIDLTPAAVGPYVVPPVNLMEHVDKDNVNLITCGGQATIPLVYAVSRVANVKYAEMVSTVSSSSAGPGTRQNIDEFTFTTSRGLEVIGGAEKGKAIIILNPAKPPILMRNTVYIAYEDGDDHQIRHSIGQMIHDVQQYVPGYRLKGEPIFDRRETPKGRLDVVILLLEVEGAGDFLPVSAGNLDIMTASAKQVGEVIAKRLIEMTSTA

SEQ ID NO: 84 - protein, 316 aa, Klebsiella pneumoniae
MRKRKVAIIGSGNIGTDLMIKILRHGQHLEMAVMVGIDPQSDGLARARRMGVATTHEGVGGLMQMAEFADIDFVFDATSAGAHIKNDAALREAKPGIRVIDLTPAAIGPYCVPVVNLAANLHQGNVNMVTCGGQATIPMVAAVSRVAKVHYAEIVASIASQSAGPGTRANIDEFTETTSQAIEKVGGAGKGKAIIVLNPAEPPLMMRDTVYVLSELASQEAIAASIAEMAAAVQAYVPGYRLKQQVQFEVIPEDKPVNLPGIGCFSGLKTAVYLEVEGAAHYLPAYAGNLDIMTSAALATAEQMAGAMHSAAGATA

SEQ ID NO: 85 - protein, 350 aa, Escherichia coli
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLDMITPQYLADLMSWGAIGARTTESQVHRELASGLFCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 86 - protein, 663 aa, Escherichia coli
MSSRKELANAIRALSMDAVQKAKSGHPGAPMGMADIAEVLWRDFLKHNPQNPSWADRDRFVLSNGHGSMLIYSLLHLTGYDLPMEELKNFRQLHSKTPGHPEVGYTAGVETTTGPLGQGIANAVGMAIAEKTLAAQFNRPGHDIVDHYTYAFMGDGCMMEGISHEVCSLAGTLKLGKLIAFYDDNGISIDGHVEGWFTDDTAMRFEAYGWHVIRDIDGHDAASIKRAVEEARAVTDKPSLLMCKTIIGFGSPNKAGTHDSHGAPLGDAEIALTREQLGWKYAPFEIPSEIYAQWDAKEAGQAKESAWNEKFAAYAKAYPQEAAEFTRRMKGEMPSDFDAKAKEFIAKLQANPAKIASRKASQNAIEAFGPLLPEFLGGSADLAPSNLTLWSGSKAINEDAAGNYIHYGVREFGMTAIANGISLHGGFLPYTSTFLMFVEYARNAVRMAALMKQRQVMVYTHDSIGLGEDGPTHQPVEQVASLRVTPNMSTWRPCDQVESAVAWKYGVERQDGPTALILSRQNLAQQERTEEQLANIARGGYVLKDCAGQPELIFIATGSEVELAVAAYEKLTAEGVKARVVSMPSTDAFDKQDAAYRESVLPKAVTARVAVEAGIADYWYKYVGLNGAIVGMTTFGESAPAELLFEEFGFTVDNVVAKAKELL

SEQ ID NO: 87 - protein, 680 aa, Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 88 - protein, 679 aa, Kluyveromyces lactis
MSQYTDIDRLAVSTIRLLAVDQVSAANSGHPGAPLGLAPAAHVIWKQMRLNPKNPEWINRDRFVLSNGHACALLYSLLHLFGYDMSIEDLKHFRHLGSKTPGHPEFELPGVEVTTGPLGQGISNAVGMAIAQANFAATYNKPDYELSDSYTYVFLGDGCLQEGVSSEASSLAGHLRLKNLIAFYDDNQITIDGNINVSFDEDVSKRYEAYGWEVLHVENGNDDLDAISKALEQAKLSDRPTLIKLTTTIGFGSLNAGSHSVHGAPLKADDVKQLKVKFGFNPEESFVVPQEVYDLYNKSTIEPGIEANKQWDALLDAYVGQFPELGAEVKRRLAGEFPEGWESKLPTYTPEDSAVASRKLSEIVLDNVFDTLPELLGGSADLTPSNLTRSKGAVDFQPPITGLGDYSGRYIRYGVREHGMGAIMNGISAFGANYRPYGGTFLNFVSYASGAVRLSALSGHPVIWVATHDSIGLGEDGPTHQPIETLAHFRAIPNLQVWRPADGNEVTAAYKVALTNKHTPAIIALSRQNLPQLQGSSVEKAVKGGYILQDVDQPDLAIVSTGSEVGIAVEAAKVLAEKNIKARIVSLPDFHSFGQQSKEYQLSVFPDGVPILSVEVLATSGWSKYAHQSFGLDRFGASGKGPAVYEKFEFTPQGIATRAEKTVEFYKGKQVISPLNTAF

SEQ ID NO: 89 - protein, 792 aa, Escherichia coli
MSNNGSSPLVLWYNQLGMNDVDRVGGKNASLGEMITNLSGMGVSVPNGFATTADAFNQFLDQSGVNQRIYELLDKTDIDDVTQLAKAGAQIRQWIIDTPFQPELENAIREAYAQLSADDENASFAVRSSATAEDMPDASFAGQQETFLNVQGFDAVLVAVKHVFASLFNDRAISYRVHQGYDHRGVALSAGVQRMVRSDLASSGVMFSIDTESGFDQVVFITSAWGLGEMVVQGAVNPDEFYVHKPTLAANRPAIVRRTMGSKKIRMVYAPTQEHGKQVKIEDVPQEQRDIFSLTNEEVQELAKQAVQIEKHYGRPMDIEWAKDGHTGKLFIVQARPETVRSRGQVMERYTLHSQGKIIAEGRAIGHRIGAGPVKVIHDISEMNRIEPGDVLVTDMTDPDWEPIMKKASAIVTNRGGRTCHAAIIARELGIPAVVGCGDATERMKDGENVTVSCAEGDTGYVYAELLEFSVKSSSVETMPDLPLKVMMNVGNPDRAFDFACLPNEGVGLARLEFIINRMIGVHPRALLEFDDQEPQLQNEIREMMKGFDSPREFYVGRLTEGIATLGAAFYPKRVIVRLSDFKSNEYANLVGGERYEPDEENPMLGFRGAGRYVSDSFRDCFALECEAVKRVRNDMGLTNVEIMIPFVRTVDQAKAVVEELARQGLKRGENGLKIIMMCEIPSNALLAEQFLEYFDGFSIGSNDMTQLALGLDRDSGVVSELFDERNDAVKALLSMAIRAAKKQGKYVGICGQGPSDHEDFAAWLMEEGIDSLSLNPDTVVQTWLSLAELKK

SEQ ID NO: 90 - protein, 61 aa, Enterobacter agglomerans
MSSKGEQPLVLWYNQLGMHDVDRVGGKNPSLGEMITNLSSLGVSVPNGFATTSYAFNLFLD

SEQ ID NO: 91 - protein, 485 aa, Saccharomyces cerevisiae
MVHLGPKKPQARKGSMADVPKELMDEIHQLEDMFTVDSETLRKVVKHFIDELNKGLTKKGGNIPMIPGWVMEFPTGKESGNYLAIDLGGTNLRVVLVKLSGNHTFDTTQSKYKLPHDMRTTKHQEELWSFIADSLKDFMVEQELLNTKDTLPLGFTFSYPASQNKINEGILQRWTKGFDIPNVEGHDVVPLLQNEISKRELPIEIVALINDTVGTLIASYYTDPETKMGVIFGTGVNGAFYDVVSDIEKLEGKLADDIPSNSPMAINCEYGSFDNEHLVLPRTKYDVAVDEQSPRPGQQAFEKMTSGYYLGELLRLVLLELNEKGLMLKDQDLSKLKQPYIMDTSYPARIEDDPFENLEDTDDIFQKDFGVKTTLPERKLIRRLCELIGTRAARLAVCGIAAICQKRGYKTGHIAADGSVYNKYPGFKEAAAKGLRDIYGWTGDASKDPITIVPAEDGSGAGAAVIAALSEKRIAEGKSLGIIGA

SEQ ID NO: 92 - protein, 485 aa, Kluyveromyces lactis
MVRLGPKKPPARKGSMADVPANLMEQIHGLETLFTVSSEKMRSIVKHFISELDKGLSKKGGNIPMIPGWVVEYPTGKETGDFLALDLGGTNLRVVLVKLGGNHDFDTTQNKYRLPDHLRTGTSEQLWSFIAKCLKEFVDEWYPDGVSEPLPLGFTFSYPASQKKINSGVLQRWTKGFDIEGVEGHDVVPMLQEQIEKLNIPINVVALINDTTGTLVASLYTDPQTKMGIIIGTGVNGAYYDVVSGIEKLEGLLPEDIGPDSPMAINCEYGSFDNEHLVLPRTKYDVIIDEESPRPGQQAFEKMTSGYYLGEIMRLVLLDLYDSGFIFKDQDISKLKEAYVMDTSYPSKIEDDPFENLEDTDDLFKTNLNIETTVVERKLIRKLAELVGTRAARLTVCGVSAICDKRGYKTAHIAADGSVFNRYPGYKEKAAQALKDIYNWDVEKMEDHPIQLVAAEDGSGVGAAIIACLTQKRLAAGKSVGIKGE

SEQ ID NO: 93 - protein, 490 aa, Aspergillus oryzae
MVGIGPKRPPSRKGSMHELPQNLLEQIKQFEDIFTVDGAKLKQIADHFVKELEKGLSVEGGNIPMNVTWVMGFPDGDEQGTFLALDMGGTNLRVCEITLTEEKGAFDITQSKYRMPEELKTGTAEELWEYIADCLQQFIESHHENEKLSKLPLGFTFSYPATQEYIDHGVLQRWTKGFDIDGVEGQDVVPPLEAILKKRGLPIKVAALINDTTGTLIASSYTDSDMKIGCIFGTGVNAAYMEHCGSVPKLAHKNLPPDMPVAINCEYGAFDNEHVVLPLTKYDIIIDRDSPRPGQQAFEKMTAGLYLGEIFRLALLDLLETRPGLIFQGQDTSQLRKPYLLDASFLAAIEDDPYENLQETQELMERKLNIKATQQELEMIRRLAELIGTRAARLSACGVAAICKKKNIESCHVGADGSVFTKYPHFKARGAQALREILDWAPNEKDKVVIMAAEDGSGVGAALIAALTLKRVKAGISCGIRDMADMQSLI

SEQ ID NO: 94 - protein, 473 aa, Zymomonas mobilis
MSSESSQGLVTRLALIAAIGGLLFGYDSAVIAAIGTPVDIHFIAPRHLSATAAASLSGMVVVAVLVGCVTGSLLSGWIGIRFGRRGGLLMSSICFVAAGFGAALTEKLFGTGGSALQIFCFFRFLAGLGIGVVSTLTPTYIAEIAPPDKRGQMVSGQQMAIVTGALTGYIFTWLLAHFGSIDWVNASGWCWSPASEGLIGIAFLLLLLTAPDTPHWLVMKGRHSEASKILARLEPQADPNLTIQKIKAGFDKAMDKSSAGLFAFGITVVFAGVSVAAFQQLVGINAVLYYAPQMFQNLGFGADTALLQTISIGVVNFIFTMIASRVVDRFGRKPLLIWGALGMAAMMAVLGCCFWFKVGGVLPLASVLLYIAVFGMSWGPVCWVVLSEMFPSSIKGAAMPIAVTGQWLANILVNFLFKVADGSPALNQTFNHGFSYLVFAALSILGGLIVARFVPETKGRSLDEIEEMWRSQK

SEQ ID NO: 95 - DNA, 39 bp, Artificial
  Primer
atctgtcauaaaacaatgcacatcgagtcacggaaatcc

SEQ ID NO: 96 - DNA, 29 bp, Artificial
  Primer
cacgcgauttaaacctccagctgcttgcc

SEQ ID NO: 97 - DNA, 34 bp, Artificial
  Primer
agtgcagguaaaacaatggcaggtaaagcacatc

SEQ ID NO: 98 - DNA, 26 bp, Artificial
  Primer
cgtgcgauttagcagccggatcaaac

SEQ ID NO: 99 - DNA, 34 bp, Artificial
  Primer
agtgcagguaaaacaatggatatcatttctgtcg

SEQ ID NO: 100 - DNA, 26 bp, Artificial
  Primer
cgtgcgauttacacttcggttaaggt

SEQ ID NO: 101 - DNA, 32 bp, Artificial
  Primer
cacgcgauttaaatgtcatagaagtccacgtg

SEQ ID NO: 102 - DNA, 32 bp, Artificial
  Primer
agtgcagguaaaacaatgaacgcggcggttgg

SEQ ID NO: 103 - DNA, 34 bp, Artificial
  Primer
atctgtcauaaaacaatggaaggaggcaggctag

SEQ ID NO: 104 - DNA, 32 bp, Artificial
  Primer
cgtgcgauttattcacgttcggcacgcagcac

SEQ ID NO: 105 - DNA, 38 bp, Artificial
  Primer
agtgcagguaaaacaatgaatgcaagcgaatttcgtcg

SEQ ID NO: 106 - DNA, 38 bp, Artificial
  Primer
atctgtcauaaaacaatgagcaccccgagcattcattg

SEQ ID NO: 107 - DNA, 34 bp, Artificial
  Primer
cacgcgauttaacgatcgctattacgacgcagtg

SEQ ID NO: 108 - DNA, 37 bp, Artificial
  Primer
agtgcagguaaaacaatggatagcaccgaagatctgg

SEQ ID NO: 109 - DNA, 32 bp, Artificial
  Primer
cgtgcgauttatttacgacccagaactgcatc

SEQ ID NO: 110 - DNA, 60 bp, Artificial
  Primer
atggaaaactttaaacatctccctgaaccgttccgcattcgtgtaggctggagctgcttc

SEQ ID NO: 111 - DNA, 59 bp, Artificial
  Primer
tcggttcgtacgtaaaggttaatcctttaatattcgccgcatatgaatatcctccttag

SEQ ID NO: 112 - DNA, 20 bp, Artificial
  Primer
cagtcatagccgaatagcct

SEQ ID NO: 113 - DNA, 25 bp, Artificial
  Primer
ggcgaattaatcggtatagcagatg

SEQ ID NO: 114 - DNA, 35 bp, Artificial
  Primer
agtgcagguaaaacaatgtcgccctctgccgtaca

SEQ ID NO: 115 - DNA, 28 bp, Artificial
  Primer
cgtgcgauttacaacttgaccgaatcaa

SEQ ID NO: 116 - DNA, 34 bp, Artificial
  Primer
agtgcagguaaaacaatgactaagctacactttg

SEQ ID NO: 117 - DNA, 24 bp, Artificial
  Primer
cgtgcgauttcagtgtatgcatgg

SEQ ID NO: 118 - DNA, 26 bp, Artificial
  Primer
acctgcacuttgtaattaaaacttag

SEQ ID NO: 119 - DNA, 25 bp, Artificial
  Primer
cacgcgaugcacacaccatagcttc

SEQ ID NO: 120 - DNA, 25 bp, Artificial
  Primer
cgtgcgauggaagtaccttcaaaga

SEQ ID NO: 121 - DNA, 26 bp, Artificial
  Primer
atgacagauttgttttatatttgttg

SEQ ID NO: 122 - DNA, 32 bp, Artificial
  Primer
agtgcagguaaaacaatgtccaagagcaaaac

SEQ ID NO: 123 - DNA, 24 bp, Artificial
  Primer
cacgcgauttaaaattccaatttc

SEQ ID NO: 124 - DNA, 31 bp, Artificial
  Primer
atctgtcauaaaacaatgtctgctccagctc

SEQ ID NO: 125 - DNA, 23 bp, Artificial
  Primer
cacgcgautcaatatctgtagtg

SEQ ID NO: 126 - DNA, 28 bp, Artificial
  Primer
agtgcagguaaaacaatggttcaatctg

SEQ ID NO: 127 - DNA, 26 bp, Artificial
  Primer
cgtgcgauttaattcttgtattgttc

SEQ ID NO: 128 - DNA, 34 bp, Artificial
  Primer
atctgtcauaaaacaatgtctaaacaatttagtc

SEQ ID NO: 129 - DNA, 29 bp, Artificial
  Primer
cacgcgauctagttatacccaaccataag

SEQ ID NO: 130 - DNA, 60 bp, Artificial
  Primer
atggcccaacaatcaccctattcagcagcgatggcagaacgtgtaggctggagctgcttc

SEQ ID NO: 131 - DNA, 58 bp, Artificial
  Primer
tcaatcgcttttcagcaacacctcttccagccactggccatatgaatatcctccttag

SEQ ID NO: 132 - DNA, 15 bp, Artificial
  Primer
gagcgccacggaatg

SEQ ID NO: 133 - DNA, 57 bp, Artificial
  Primer
atgttccagcaagaagttaccattaccgctccgaacggtgtaggctggagctgcttc

SEQ ID NO: 134 - DNA, 60 bp, Artificial
  Primer
ttactcgagttccgccatcagtttaaccagatgttcaacccatatgaatatcctccttag

SEQ ID NO: 135 - DNA, 21 bp, Artificial
  Primer
actggccgtcgttttacaacg

SEQ ID NO: 136 - DNA, 23 bp, Artificial
  Primer
catggtcatagctgtttcctgtg

SEQ ID NO: 137 - DNA, 36 bp, Artificial
  Primer
caggaaacagctatgaccatgaattatcagaacgac

SEQ ID NO: 138 - DNA, 41 bp, Artificial
  Primer
tgtaaaacgacggccagtttacccgcgacgcgcttttactg

SEQ ID NO: 139 - DNA, 16 bp, Artificial
  Primer
gactgggaaaaccctg

SEQ ID NO: 140 - DNA, 22 bp, Artificial
  Primer
tgtaaaacgacggccagtttac

SEQ ID NO: 141 - DNA, 30 bp, Artificial
  Primer
gccgtcgttttacaatgtcctcacgtaaag

SEQ ID NO: 142 - DNA, 39 bp, Artificial
  Primer
gagccattgttggacatacgacgttacagcagttctttt

SEQ ID NO: 143 - DNA, 39 bp, Artificial
  Primer
aaaagaactgctgtaacgtcgtatgtccaacaatggctc

SEQ ID NO: 144 - DNA, 34 bp, Artificial
  Primer
gcgattaagttgggtaacgccagggttttcccag

SEQ ID NO: 145 - DNA, 6979 bp, Artificial
  pGAL1-cre
cggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgacaattccaaccttacccaagagttcgccaaactcagacatcactttangcaaaaccgcgccgtgcttcttcctcggtggcattcatcacgaaatgttcagcactacgcatacttttgacaggaaacgcaacggatattgagtcaatatcaggcattctatcgctcagctttacagtgacaatgacggctggcgactgaatattagtgcttacagacagcactacatattttccgtcgatgttgaaatcctttctcatatgtcaccataaatatcaaataattatagcaatcatttacgcgttaatggctaatcgccatcttccagcaggcgcaccattgcccctgtttcactatccagggtacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatttaacactcagataatggttttaagtaaagtgtacaggatcggctctgcccctcgacggtatcgataagcttgatatcgaattcctgcagcccgggggatccactagttctagaatccggggttttttctccttgacgttaaagtatagaggtatattaacaattttttgttgatacttttattacatttgaataagaagtaatacaaaccgaaaatgttgaaagtattagttaaagtggttatgcagtttttgcatttatatatctgttaatagatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtactagagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatgcatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgg

SEQ ID NO: 146 - DNA, 2417 bp, Artificial
  TDP operon
tttacactttatgcttccggctcgtatgttgtgtggaattgtgagcggataacaatttcacacaggaaacagctatgaccatggatgacaaaggcaacaaaggcagcagcaaacgtgaagcggccaccgaaagcggcaaaaccgccgtggtttttagcctgaaaaacgaagtgggcggtctggtgaaagcgctgcgtctgtttcaggaaaaacgtgtgaacatggtgcatattgaaagccgtaaaagccgtcgccgtagcagcgaagtggaaatttttgtggattgcgaatgcggcaaaaccgaatttaacgaactgattcagctgctgaaatttcagaccaccattgtgaccctgaacccgccggaaaacatttggaccgaagaggaagagctggaagatgtgccgtggtttccgcgtaaaattagcgaactggataaatgcagccatcgtgtgctgatgtatggcagcgaactggatgcggatcatccgggctttaaagataacgtgtatcgtcagcgtcgcaaatattttgtggatgtggcgatgggctataaatatggccagccgattccgcgtgtggaatataccgaagaggaaaccaaaacctggggcgtggtttttcgtgaactgagcaaactgtatccgacccatgcgtgccgtgaatatctgaaaaactttccgctgctgaccaaatattgcggctatcgtgaagataacgtgccgcagctggaagatgtgagcatgtttctgaaagaacgtagcggctttaccgtgcgtccggtggcgggctatctgagcccgcgtgattttctggcgggcctggcgtatcgtgtgtttcattgcacccagtatattcgtcatggcagcgatccgctgtataccccggaaccggatacctgccatgaactgctgggccatgttccgctgctggccgatccgaaatttgcgcagtttagccaggaaattggcctggcgagcctgggcgcgagcgatgaagatgtgcagaaactggcgacctgctatttctttaccattgaatttggcctgtgcaaacaggaaggccagctgcgtgcctatggtgcgggcctgctgagcagcattggcgaactgaaacatgcgctgagcgataaagcgtgcgtgaaagcgtttgatccgaaaaccacctgcctgcaggaatgcctgattaccacctttcaggaagcgtattttgtgagcgaaagctttgaagaggcgaaagaaaaaatgcgaaaagcattacccgtccgtttagcgtgtattttaacccgtatacccagagcattgaaattctgaaagatacccgtagcattgaaaacgtggttcaggatctgcgtataataagccgcggaggattacactatggatatcatttctgtcgccttaaagcgtcattccactaaggcatttgatgccagcaaaaaacttaccccggaacaggccgagcagatcaaaacgctactgcaatacagcccatccagcaccaactcccagccgtggcattttattgttgccagcacggaagaaggtaaagcgcgtgttgccaaatccgctgccggtaattacgtgttcaacgagcgtaaaatgcttgatgcctcgcacgtcgtggtgttctgtgcaaaaaccgcgatggacgatgtctggctgaagctggttgttgaccaggaagatgccgatggccgctttgccacgccggaagcgaaagccgcgaacgataaaggtcgcaagttcttcgctgatatgcaccgtaaagatctgcatgatgatgcagagtggatggcaaaacaggtttatctcaacgtcggtaacttcctgctcggcgtggcggctctgggtctggacgcggtacccatcgaaggttttgacgccgccatcctcgatgcagaatttggtctgaaagagaaaggctacaccagtctggtggttgttccggtaggtcatcacagcgttgaagattttaacgctacgctgccgaaatctcgtctgccgcaaaacatcaccttaaccgaagtgtaataagccgcggaggattacactgtgtaataagccgcggaggattacactatggcaggtaaagcacatcgtctgagcgcagaagaacgcgatcagctgctgccgaatctgcgtgcagttggttggaatgaactggaaggtcgtgatgcaatctttaaacagttccacttcaaagatttcaaccgtgcctttggttttatgacccgtgttgcactgcaggcagaaaaactggatcatcatccggaatggtttaacgtgtataacaaagtgcatattaccctgagcacccatgaatgtgcaggtctgagcgaacgtgatattaatctggcaagctttattgaacaggttgcagttagcatgacctaatgagtttgatccggctgctaa

SEQ ID NO: 147 - DNA, 2008 bp, Artificial
  GPS operon
tttacactttatgcttccggctcgtatgttgtgtggaattgtgagcggataacaatttcacacaggaaaacatatgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtgatcggtctgtcaaaaattaaccgcattgtgcagttctttgcccagcgtccgcaggtgcaggaacgtctgacgcagcaaattcttattgcgctacaaacgctgctgggcaccaataacgtggctgtctcgatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactaataagccgcggaggattacactatgaacgcggcggttggccttcggcgccgcgcgcgattgtcgcgcctcgtgtccttcagcgcgagccaccggctgcacagcccatctctgagtgctgaggagaacttgaaagtgtttgggaaatgcaacaatccgaatggccatgggcacaactataaagttgtggtgacaattcatggagagatcgatccggttacaggaatggttatgaatttgactgacctcaaagaatacatggaggaggccattatgaagccccttgatcacaagaacctggatctggatgtgccatactttgcagatgttgtaagcacgacagaaaatgtagctgtctatatctgggagaacctgcagagacttcttccagtgggagctctctataaagtaaaagtgtatgaaactgacaacaacattgtggtctacaaaggagaataataagccgcggaggattacactatggaaggaggcaggctaggttgcgctgtctgcgtgctgaccggggcttcccggggcttcggccgcgccctggccccgcagctggccgggttgctgtcgcccggttcggtgttgcttctaagcgcacgcagtgactcgatgctgcggcaactgaaggaggagctctgtacgcagcagccgggcctgcaagtggtgctggcagccgccgatttgggcaccgagtccggcgtgcaacagttgctgagcgcggtgcgcgagctccctaggcccgagaggctgcagcgcctcctgctcatcaacaatgcaggcactcttggggatgtttccaaaggcttcctgaacatcaatgacctagctgaggtgaacaactactgggccctgaacctaacctccatgctctgcttgaccaccggcaccttgaatgccttctccaatagccctggcctgagcaagactgtagttaacatctcatctctgtgtgccctgcagcccttcaagggctggggactctactgtgcagggaaggctgcccgagacatgttataccaggtcctggctgttgaggaacccagtgtgagggtgctgagctatgccccaggtcccctggacaccaacatgcagcagttggcccgggaaacctccatggacccagagttgaggagcagactgcagaagttgaattctgagggggagctggtggactgtgggacttcagcccagaaactgctgagcttgctgcaaagggacaccttccaatctggagcccacgtggacttctatgacatttaa

SEQ ID NO: 148 - DNA, 11909 bp, Artificial
  Plasmid 380
cattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtcagctgaagcttcgtacgctgcaggtcgacaacccttaatataacttcgtataatgtatgctatacgaagttattaggtctagagatccgcaggctaaccggaacctgtattatttagtttatgctacgttaaataaagacctttcgttcacataactgaatgtgtaatggccttgagatttcaagcataccaagttggtggagacggggtcgttacaaaagactctttaagccaagatttccttgacagccttggcgatagcatcgccaacctcagtggtagagttagaaccaccaaggtcaccggttctgacacctgcatccaagacatttctaacagcttcttcaagagccctaccttcttcaaccaaatccaaggataacttcaacatcatagctgcagataagatggtagcaattgggttaaccttgtttgctggtaaatctggggcagaaccatgacatggttcgtacaaaccgaatgccttgttagtgtcaggtagggaagctagagatgcagaaggtaataaacccaaagaacctggaataacagaggcttcatcggagataatatcaccaaacatgttgttggtaataacaacaccgtttagcttagttggtgatttaaccaaaatcatagcagcagagtcgatcaattggtgctgaacagttaattgtgggaactcagtcttgatggtttcttcaacagtctttctccacaatctggaagaggcaagcacgttagccttgtcaagagaccagattggtaatggtgggttttgttgcaatgccaagaaagcagccattcttgtaattctttgaacttcaggaacactgtatttctcagagtcccaagcaactccgtcaccttcatcttcttttctttcaccaaagtagataccaccaaccaattctctaacgacgacgaaatcggtaccctttgcatattcaggcttcaaaggagaaagatctagtaaagaatcagaagcaaagttacatggtctcaagttggcgtatagacccaattcctttctgatcttcaatagaccttgttctggtctaactgcgcccgtaccccattttggaccaccaacagcacctagtaagacagcatcggctttcttagaggcttctagagcttcatctggtaaaggagtgccagtggcatcgatggcagcacccccgatcaagtgatgttggaaattgaacttaatttctggacggacttcagcaatggcattcaagaccttaatagcttcgtcagtaacttctttaccgacgtgatcacccggtaggacaacgatattcttagacatggtataatctgtgtagtgtgggatactttttacttcttcaaataggtatcaacaataaaaataaatcgaagcaaatgtaggaatgcgttaaagcagatgtacttacttagagtacatagatatatttatataattcaatatataaaagtatatgaagcatctgatgttgaacctgtcatgactctaaactggaatgggtagttatgcttctgaaggttttccgttagaaaatgagtgcagggatcaggcccggaaccggttttagcctgaaaggaaaagaaaaaaatgcggtgattactagcacgtgactgcgctgaattggaatccaccacaagtacagcgaatggcaagagggaaggggaatatattgtgcctggcggtagtctgctatatcaaatgttaaacataactaaatgcagtagtgaatagagtaatgtatttctacatatgtgggtagggccaatgggagcccgatgtctattgtacagatattttccttttatagttaatcaattcaacttggaaatcattgagtcgatatctacttggataactggagccgtggcagcgtttgaaatttgacgagtcttcttgttttgttcgcgcattttcagtttattcaaacgggagatggcaccgaatccactgacttctctgttcaatttgaatggtaataggaagaaagtacatttcgagaaattttcgcccaacgtcgaaatctcatggatgatatcctcgatacaaatgacaccttcatcacctaatttctcttcaacgatattgttgtcattcaagattatctcttttggttctttatcttctggcctttgccacattattctagatctcttttggattaaagaacggatagatgcaagtgaaggttgtccgataacaatatatggtgatacaagtttcaacaatgggtacacgtccttggtaagtttgatgaaaacaccagtgttcaactcaacaagtctcaaaagagataacactttgtaagcctttgctgggatcttcacagcgagctctcgagaacccttaatataacttcgtataatgtatgctatacgaagttattaggtaggtgatatcagatccactagtggcctatgcacccaattcgccctatagtgagtcgtattacgcgcgctcactggccgtcgttttacaacgtcgtgactgggaaaaccctggcgttacccctgcaggactagtgctgaggcattaattgtgtccgcgtttctaaggccacttttcaatgaaacggatattgatatgctagtaaaaggacgagctcaagagcgaaaatataagtaaagaattcgagtgcacttgtctccatgcagcaagatttcatatgagtcttttttatctttttactttttacattacacgatatgcactttatgaaaatttaacgaggttggaagccggataatcaaccaaaatcaggcacgaaggcacactcgtatatgcatgttgttgaaactctgttacgctgaactaacaatcacacatgtagaggtcaccgggaaaagttgcgaccccatggaaggtcgatctcttcgtttggctttgcttggctggcggcattgcgcttcttcgcttatacccgtctcttgacgctcgagctcgttcattgagatacctttattcttgcacattttctggcttttttcgctactcgggtacatgtaatcatgcacacagaaggtgctgtagggtgaaagttcctttgtgctgtcgtttgtttttaatgccaaactttccggtgatcaataaccacctcattaagacctcagcgcggccgcaaatttaaataaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtggggatttaaatgcggccgcgctgagggtttaatcgagatctttgtgttcggttacccggctcagatcctaacttggtatgtttattcgtataagttactgttgtccacaggcaatactctgcagaaaattaaaacggcattaatgctaggacaaccagaattgttactactgtatgtgcgatagttgataactgcaacattatgcccggtatattctcaaaaaaccctattactgcatacgaagaaatcgcaagagaaatctttcggtttggaaaagctcactgtgaggttccttggagccaatagtaatacagcacaatccaaggaaaaatctggcctatatgcaaggaaggagagatagtcaaaagcattctttcccctagaagttggtgcatatatggcatcgttaaaacatattacccccaaaatttcttctctaaacgatgtgcttggcctttgttttggtttttgatgtcggtcgtttgaggccccttgcggaaaatcgagatcgccgaatggcacgcgagggaagggaaataaggtttaaaggcactgaaacaataggcaagaagtaggcgagagccgacatacgagactattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattcatgatggcatagcaatagcttgtttttcctctaaaggtttttcgaccacgcctggatcggcaagagtagaagtatcacctaagttagacgtatctccggcggcgatctttctaagtatacgtctcataatctttccagatcttgtcttaggcaatgaatctgtccaatgcaaaacatctggtgtggctaaaggccctatttcctttcgaacccaatttctgacctctgcgtatagctctggagatggttcctcaccatggttgagtgtaacatatgcgtatatggcttgacccttgatagcgtgtgggatgccaacgactgcagcttcagcaatcttaggatgagcaactaaagctgactcaatctcagctgtacccagacgatgtccagatacgttaaggacgtcatctactctgccggtaatccagtagtacccatcttcgtctcttctcgcaccgtcaccgctgaaatacatgtttttgaaagttgagaaataggtttgttcgaatctttcgtgatcaccaaacaatgttctagcctgaccaggccaggaatcagttatcactagattgccttctgttgccccctcttgtggatgcccttcgttgtctactaaagcaggttggacgccgaagaaaggcctagtagcagacccagccttcaattctattgcacctggaagaggtgtaatcatgaatccacctgtttcagtttgccaccatgtatccactactggacacttttccttaccaatctttttccaataccattcccaagcttctggattgattggctcgccaacggatcctaagatacgtaggctacttctatctgttccctcaatcgccttatctccttcggccattagcgctctaatggcggttggggctgtgtacaatatgttcacttggtgtttatcgaccacttgacacatcctggctggggttggccagtttggtactccttcaaacatcaaagtggttgcgccgcatgccagtggaccgtaaagcaggtaactatgaccagtaacccaaccgacatcagcggtacaccagtagatatcacctggatggtaatcaaaaacgtacttaaaggttgttgcggcgtatacgagataaccacctgttgtatgcaaaacaccttttggtttgccagttgacccagatgtatacaagatgaacaatggatcttccgcattcattgcttctggttggtgttctggacttgctttctcaataagatctctccaccacaaatccctaccttcctgccagtctatatcagagccggtacgctttagtacaattacatgctccacactcgtaacgtttgggtttttcaacgcgtcatcaacgtttttcttcaatgggatggatctacctgctctaacaccttcgtcggcagtgataactaatctacttgatgagtcaatgattctcccagcaacggcttcaggggagaatccgccaaagataacgctatggactgctcctattctggcacaggccaacattgccacagctgcctctgggaccataggcatgtagattgccacaacatcgccctttttgatacccaggtccagtaaagtattcgcgaatctgcaaacatctctgtgtaactctctgtaggagatgtgcttagattgagaagtatcatcaccttcccaaatgattgccgttctgtcaccattttcctggaggtgtcgatccaagcaattagcagctaggtttagagtgccgtcctcgtaccacttgattgacacatttccaggagcaaaggatgtgtttttgaccttttgatatggagtaatccaatcgagtatcttaccttgctcgccccagaaagtatcaggatcgttgatagactgcttgtacttcgtttcgtattgttcagggttgattaagcacctgtcagcgatattcgcaggaatagcatgtttgtgtgtttgtgacattgttttacctgcactacctgcactttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctatggtgtgtgcggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaatctgtcatatctgtcataaaacaatgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtgaatcgcgtg

SEQ ID NO: 149 - DNA, 2661 bp, Artificial
  PK18
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg

SEQ ID NO: 150 - DNA, 435 bp, Artificial
  Codon optimized sequence
atgaacgcggcggttggccttcggcgccgcgcgcgattgtcgcgcctcgtgtccttcagcgcgagccaccggctgcacagcccatctctgagtgctgaggagaacttgaaagtgtttgggaaatgcaacaatccgaatggccatgggcacaactataaagttgtggtgacaattcatggagagatcgatccggttacaggaatggttatgaatttgactgacctcaaagaatacatggaggaggccattatgaagccccttgatcacaagaacctggatctggatgtgccatactttgcagatgttgtaagcacgacagaaaatgtagctgtctatatctgggagaacctgcagagacttcttccagtgggagctctctataaagtaaaagtgtatgaaactgacaacaacattgtggtctacaaaggagaataa

SEQ ID NO: 151 - DNA, 789 bp, Artificial
  Codon-optimized sequence
atggaaggaggcaggctaggttgcgctgtctgcgtgctgaccggggcttcccggggcttcggccgcgccctggccccgcagctggccgggttgctgtcgcccggttcggtgttgcttctaagcgcacgcagtgactcgatgctgcggcaactgaaggaggagctctgtacgcagcagccgggcctgcaagtggtgctggcagccgccgatttgggcaccgagtccggcgtgcaacagttgctgagcgcggtgcgcgagctccctaggcccgagaggctgcagcgcctcctgctcatcaacaatgcaggcactcttggggatgtttccaaaggcttcctgaacatcaatgacctagctgaggtgaacaactactgggccctgaacctaacctccatgctctgcttgaccaccggcaccttgaatgccttctccaatagccctggcctgagcaagactgtagttaacatctcatctctgtgtgccctgcagcccttcaagggctggggactctactgtgcagggaaggctgcccgagacatgttataccaggtcctggctgttgaggaacccagtgtgagggtgctgagctatgccccaggtcccctggacaccaacatgcagcagttggcccgggaaacctccatggacccagagttgaggagcagactgcagaagttgaattctgagggggagctggtggactgtgggacttcagcccagaaactgctgagcttgctgcaaagggacaccttccaatctggagcccacgtggacttctatgacatttaa

SEQ ID NO: 152 - DNA, 735 bp, Artificial
  Codon-optimized sequence
atggctgctgctgcagctgctggtgaagctagaagagttttggtttatggtggtagaggtgctttgggttctagatgtgttcaagcttttagagctagaaattggtgggttgcttctgttgatgttgtcgaaaatgaagaagcctccgcttctattatcgttaagatgactgattccttcaccgaacaagctgatcaagttactgctgaagtaggtaagttgttgggtgaagaaaaggttgatgctattttgtgtgttgctggtggttgggctggtggtaatgctaaatctaaatctttgttcaagaactgcgacttgatgtggaagcaatctatttggacttccaccatctcttctcatttggctaccaaacatttgaaagaaggtggtttgttgactttggctggtgctaaagctgctttggatggtactccaggtatgattggttatggtatggctaaaggtgctgttcatcaattgtgtcaatcattggctggtaagaattctggtatgccaccaggtgctgctgctattgctgttttgccagttactttggatactccaatgaacagaaagtctatgccagaagctgatttctcttcttggactccattggaattcttggttgaaactttccatgattggatcaccggtaagaatagaccatcttctggttccttgattcaagttgttactactgaaggtagaaccgaattgactccagcttacttctaa

SEQ ID NO: 153 - DNA, 315 bp, Artificial
  Codon-optimized sequence
atggctggtaaagctcatagattgtctgctgaagaaagagatcaattattgccaaacttgagagctgttggttggaatgaattggaaggtagagatgccattttcaagcaattccacttcaaggatttcaacagagctttcggtttcatgactagagttgctttacaagccgaaaagttggatcatcatccagaatggtttaacgtctacaacaaggttcatatcaccttgtctactcatgaatgtgctggtttgtctgaaagagatattaacttggcctccttcattgaacaagttgctgtttctatgacttaa

SEQ ID NO: 154 - DNA, 1476 bp, Artificial
  Codon-optimized sequence
atgcagcctgcaatgatgatgtttagcagcaaatattgggcacgtcgtggttttagcctggatagcgcagttccggaagaacatcagctgctgggtaatctgaccgtgaataaaagcaatagcggcaaaaacgatgataaaaaaggcaataaaggtagcagccgtagcgaaaccgcaccggatagcggtaaaaccgcagttgtgtttagcctgcgtaatgaagttggtggtctggttaaagcactgaaactgtttcaagaaaaacatgtgaacatggtgcatatcgaaagccgtaaaagccgtcgtcgtagcagcgaagttgaaatttttgttgattgcgaatgcggcaaaaccgaatttaatgaactgatccagctgctgaaattccagaccacaattgttaccctgaatccgcctgaaaacatttggaccgaagaagaagaactggaagacgttccgtggtttccgcgtaaaattagcgaactggataaatgtagccatcgtgttctgatgtatggtagtgaactggatgcagatcatccgggttttaaagataatgtttatcgtcagcgtcgcaaatatttcgttgatgttgcaatgagctataaatacggtcagccgattccgcgtgttgaatatacggaagaagaaaccaaaacctggggtgttgtttttcgtgaactgagccgtctgtatccgacccatgcatgtcaagaatatctgaaaaactttccgctgctgaccaaatattgtggttatcgtgaagataacgttccgcagctggaagatgttagcatgtttctgaaagaacgtagcggttttgcagttcgtccggttgcaggttatctgagtccgcgtgattttctggcaggtctggcatatcgtgtttttcattgtacccagtatgttcgccatagcagcgatccgctgtatactccggaaccggatacctgtcatgaactgctgggtcatgtgccgctgctggcagatccgaaatttgcacagtttagccaagaaattggtctggcaagcctgggtgcaagtgatgaagatgttcagaaactggcaacctgctattttttcaccattgaatttggcctgtgcaaacaagagggtcagctgcgtgcatatggtgcaggtctgctgagcagcattggtgaactgaaacatgcactgagcgataaagcatgtgttaaagcatttgatccgaaaaccacctgtctgcaagaatgtctgattaccacctttcaagaagcctatttcgttagcgagagctttgaagaggcaaaagaaaaaatgcgcgaatttgccaaaagcattacccgtccgtttagcgttcattttaatccgtatacccagagcgttgaagtgctgaaagatagccgtagcattgaaagcgttgttcaggatctgcgtagcgatctgaataccgtttgtgatgcactgaacaaaatgaatcagtatctgggctgataa

SEQ ID NO: 155 - DNA, 435 bp, Artificial; identical to SEQ ID NO: 150
  Codon-optimized sequence

SEQ ID NO: 156 - DNA, 789 bp, Artificial; identical to SEQ ID NO: 151
  Codon-optimized sequence

SEQ ID NO: 157 - DNA, 735 bp, Artificial
  Codon-optimized sequence
atggcagctgcagcagcagccggtgaagcacgtcgtgttctggtttatggtggtcgtggtgcactgggtagccgttgtgttcaggcatttcgtgcacgtaattggtgggttgcaagcgttgatgttgttgaaaatgaagaagcaagcgccagcattattgtgaaaatgaccgatagctttaccgaacaggcagatcaggttaccgcagaagttggtaaactgctgggtgaagaaaaagttgatgcaattctgtgtgttgccggtggttgggcaggcggtaatgcaaaaagcaaaagcctgtttaaaaactgcgacctgatgtggaaacagagcatttggaccagcaccattagcagccatctggcaaccaaacatctgaaagaaggtggtctgctgaccctggcaggcgcaaaagcagcactggatggtacaccgggtatgattggttatggtatggcaaaaggtgcagttcatcagctgtgtcagagcctggcaggtaaaaatagcggtatgcctccgggtgcagcagccattgcagttctgccggttaccctggataccccgatgaatcgtaaaagcatgccggaagcagattttagcagctggacaccgctggaatttctggttgaaacctttcatgattggatcaccggtaaaaatcgtccgagcagcggtagcctgattcaggttgttaccaccgaaggtcgtaccgaactgacaccggcatatttttga

SEQ ID NO: 158 - DNA, 336 bp, Artificial
  Codon-optimized sequence
atggcaggtaaagcacatcgtctgagcgcagaagaacgcgatcagctgctgccgaatctgcgtgcagttggttggaatgaactggaaggtcgtgatgcaatctttaaacagttccacttcaaagatttcaaccgtgcctttggttttatgacccgtgttgcactgcaggcagaaaaactggatcatcatccggaatggtttaacgtgtataacaaagtgcatattaccctgagcacccatgaatgtgcaggtctgagcgaacgtgatattaatctggcaagctttattgaacaggttgcagttagcatgacctaatgagtttgatccggctgctaa

SEQ ID NO: 159 - DNA, 1461 bp, Artificial
  Codon-optimized sequence
atgaacgcaagcgattttcgtcgtcgtggtaaagaaatggttgattatatggccgattatctggaaggtattgaaggtcgtcaggtttatccggatgttcagcctggttatctgcgtccgctgattccggcaaccgcaccgcaagaaccggatacctttgaagatattctgcaggatgtggaaaaaatcattatgcctggtgttacccattggcatagcccgtatttctttgcatattttccgaccgcaagcagctatccggcaatgctggcagatatgctgtgtggtgcaattggttgtattggttttagctgggcagcaagtccggcatgtaccgaactggaaaccgttatgatggattggctgggtaaaatgctgcagctgccggaagcatttctggcaggcgaagccggtgaaggtggtggtgttattcagggtagcgcaagcgaagcaaccctggttgcactgctggcagcacgtaccaaagttgttcgtcgtctgcaggcagcatcaccgggtctgacccagggtgcagttctggaaaaactggttgcatatgccagcgatcaggcacatagcagcgttgaacgtgcaggtctgattggtggtgtgaaactgaaagcaattccgagtgatggtaaatttgcaatgcgtgcaagtgcactgcaagaagcactggaacgcgataaagcagccggtctgattccgttttttgttgttgcgaccctgggtacaaccagctgttgcagctttgataatctgctggaagttggtccgatttgccatgaagaggatatttggctgcatgttgatgcagcatatgcaggtagcgcatttatttgtccggaatttcgtcatctgctgaatggtgttgaatttgccgatagctttaacttcaatccgcataaatggctgctggtgaattttgattgtagcgcaatgtgggttaaacgtcgtaccgatctgaccggtgcatttaaactggatccggtgtatctgaaacatagccatcagggtagtggtctgattaccgattatcgtcattggcaactgccgctgggtcgtcgttttcgtagcctgaaaatgtggtttgtgtttcgtatgtatggtgttaaaggactgcaggcctatattcgtaaacatgttcagctgagccatgaatttgaagcatttgtgctgcaggatccgcgttttgaagtttgtgcagaagttaccctgggcctggtttgctttcgtctgaaaggtagtgatggtctgaatgaagccctgctggaacgtattaatagcgcacgtaaaattcatctggttccgtgtcgtctgcgtggtcagtttgttctgcgttttgcaatttgtagccgtaaagttgaaagcggtcatgttcgtctggcatgggaacatattcgtggtctggcagcagagctgctggctgcagaagaaggtaaagcagaaatcaaaagctaa

SEQ ID NO: 160 - DNA, 1038 bp, Artificial
  Codon-optimized sequence
atgtgtagccaagaaggtgaaggttatagcctgctgaaagaatatgccaatggttttatggttagccaggttctgtttgcagcatgtgaactgggtgtttttgaactgctggcagaagcactggaaccgctggatagcgcagcagttagcagccatctgggtagcagtccgcagggcaccgaactgctgctgaatacctgtgttagcctgaaactgctgcaggcagatgttcgtggtggtaaagcagtttatgcaaataccgaactggcaagcacctatctggttcgtggtagtccgcgtagccagcgtgatatgctgctgtatgcaggtcgtaccgcatatgtttgttggcgtcatctggccgaagcagttcgtgaaggtcgtaatcagtatctgaaagcatttggtattccgagcgaagaactgtttagcgcaatttatcgtagcgaagatgaacgtctgcagttcatgcagggtctgcaggatgtttggcgtctggaaggtgcaaccgttctggcagcatttgatctgagcccgtttccgctgatttgtgatctgggtggtggtagcggtgcactggcaaaagcatgcgttagcctgtatccgggttgtcgtgcaattgtttttgatattccgggtgttgttcagattgccaaacgtcattttagcgcatcagaagatgagcgcattagctttcatgaaggcgattttttcaaagatgcactgccggaagcagatctgtatattctggcacgtgttctgcatgattggaccgatgcaaaatgtagccatctgctgcaacgtgtttatcgtgcctgtcgtaccggtggtggcattctggttattgaaagcctgctggataccgatggtcgtggtccgctgaccaccctgctgtatagtctgaatatgctggttcagaccgaaggtcgtgaacgtacaccggcagaatatcgtgcactgctgggtccggcaggttttcgtgatgttcgttgtcgtcgcaccggtggcacctatgatgcagtgctggcacgtaaataa

SEQ ID NO: 161 - DNA, 1041 bp, Artificial
  Codon-optimized sequence
atggatagcaccgaagatctggattatccgcagattatctttcagtatagcaatggttttctggtgagcaaagttatgtttaccgcatgtgaactgggtgtttttgatctgctgctgcagagcggtcgtccgctgagcctggatgttattgcagcacgtctgggcaccagcattatgggtatggaacgtctgctggatgcatgtgttggtctgaaactgctggcagttgaactgcgtcgtgaaggtgcattttatcgtaataccgaaatcagcaacatctatctgaccaaaagcagcccgaaaagccagtatcatatcatgatgtattatagcaacaccgtgtatctgtgctggcattatctgaccgatgcagtgcgtgaaggtcgtaatcagtatgaacgtgcatttggtattagcagcaaagacctgtttggtgcacgttatcgtagcgaagaagaaatgctgaaatttctggcaggtcagaatagcatttggagcatttgtggtcgtgatgttctgaccgcatttgatctgagcccgtttacccagatttatgatctgggtggtggtggcggtgcactggcacaagaatgtgtttttctgtatccgaattgcaccgtgaccatctatgatctgccgaaagttgttcaggttgcaaaagaacgtctggttccgcctgaagaacgtcgtattgcatttcatgaaggcgatttcttcaaagatagcattccggaagcagatctgtatatcctgagcaaaattctgcatgattgggatgacaaaaaatgtcgtcagctgctggccgaagtttataaagcatgtcgtcctggcggtggtgttctgctggttgaaagcctgctgagcgaagatcgtagcggtccggttgaaacccagctgtatagcctgaatatgctggttcagaccgaaggtaaagaacgtaccgcagttgaatatagcgaactgctgggtgcagcaggttttcgtgaagttcaggttcgtcgtaccggtaaactgtatgatgcagttctgggtcgtaaataa

SEQ ID NO: 162 - DNA, 39 bp, Artificial
  Primer
atctgtcauaaaacaatggatgacaaaggcaacaaaggc

SEQ ID NO: 163 - DNA, 30 bp, Artificial
  Primer
cacgcgauttatacgcagatcctgaaccac

SEQ ID NO: 164 - DNA, 34 bp, Artificial
  Primer
atctgtcauaaaacaatgattagcaccgaaagcg

SEQ ID NO: 165 - DNA, 27 bp, Artificial
  Primer
cacgcgauttagctgctgcgattttcg

SEQ ID NO: 166 - DNA, 36 bp, Artificial
  Primer
atctgtcauaaaacaatggaactggaagatgttccg

SEQ ID NO: 167 - DNA, 33 bp, Artificial
  Primer
cacgcgauttaggtatctttcaggatctcgatg

SEQ ID NO: 168 - DNA, 35 bp, Artificial
  Primer
agtgcaggtaaaacaatggtcaagttgttcccatc

SEQ ID NO: 169 - DNA, 32 bp, Artificial
  Primer
cgtgcgautcaaattctggcatcttgaatttc

SEQ ID NO: 170 - DNA, 29 bp, Artificial
  Primer
atctgtcauaaaacaatggctgctgctgc

SEQ ID NO: 171 - DNA, 27 bp, Artificial
  Primer
cacgcgauttagaagtaagctggagtc

SEQ ID NO: 172 - DNA, 32 bp, Artificial
  Primer
cgtgcgauttattctcctttgtagaccacaat

SEQ ID NO: 173 - DNA, 34 bp, Artificial
  Primer
agtgcagguaaaacaatgggtagcagcgaagatc

SEQ ID NO: 174 - DNA, 32 bp, Artificial
  Primer
cgtgcgauttatttacgtgccaggattgcatc

SEQ ID NO: 175 - protein, 448 aa, Homo sapiens
MDDKGNKGSSKREAATESGKTAVVFSLKNEVGGLVKALRLFQEKRVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRINRVHSSALTEKEGVRQPEV

SEQ ID NO: 176 - protein, 315 aa, Homo sapiens
MELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDT

SEQ ID NO: 177 - protein, 497 aa, Schistosoma mansoni
MISTESDLRRQLDENVRSEADESTKEECPYINAVQSHHQNVQEMSIIISLVKNMNDMKSIISIFTDRNINILHIESRLGRLNMKKHTEKSEFEPLELLVHVEVPCIEVERLLEELKSFSSYRIVQNPLMNLPEAKNPTLDDKVPWFPRHISDLDKVSNSVLMYGKELDADHPGFKDKEYRKRRMMFADIALNYKWGQQIPIVEYTEIEKTTWGRIYRELTRLYKTSACHEFQKNLGLLQDKAGYNEFDLPQLQVVSDFLKARTGFCLRPVAGYLSARDFLSGLAFRVFYCTQYIRHQADPFYTPEPDCCHELLGHVPMLADPKFARFSQEIGLASLGTSDEEIKKLATCYFFTIEFGLCRQDNQLKAYGAGLLSSVAELQHALSDKAVIKPFIPMKVINEECLVTTFQNGYFETSSFEDATRQMREFVRTIKRPFDVHYNPYTQSIEIIKTPKSVAKLVQDLQFELTAINESLLKMNKEIRSQQFTTNKIVTENRSS

SEQ ID NO: 178 - protein, 207 aa, Bos taurus
MSTPSIHCLKPSPLHLPSGIPGSPGRQRRHTLPANEFRCLTPEDAAGVFEIEREPFISVSGNCPLNLDEVRHFLTLCPELSLGWFVEGRLVAFIIGSLWDEERLTQESLTLHRPGGRTAHLHALAVHHSFRQQGKGSVLLWRYLQHAGGQPAVRRAVLMCEDALVPFYQRFGFHPAGPCAVVVGSLTFTEMHCSLRGHAALRRNSDR

SEQ ID NO: 179 - DNA, 948 bp, Artificial
  Codon-optimized sequence
atggaactggaagatgttccgtggtttccgcgtaaaattagcgaactggataaatgtagccatcgtgttctgatgtatggtagtgaactggatgcagatcatccgggttttaaagataatgtttatcgtcagcgtcgcaaatatttcgttgatgttgcaatgggttacaaatatggtcagccgattccgcgtgttgaatataccgaagaagaaaccaaaacctggggtgttgtttttcgtgaactgagcaaactgtatccgacccatgcatgtcgtgaatatctgaaaaactttccgctgctgaccaaatattgtggttatcgtgaagataacgttccgcagctggaagatgtgagcatgtttctgaaagaacgtagcggttttaccgttcgtccggttgcaggttatctgagtccgcgtgattttctggcaggtctggcatatcgtgtttttcattgtacccagtatattcgccatggtagcgatccgctgtatactccggaaccggatacctgtcatgaactgctgggtcatgtgccgctgctggcagatccgaaatttgcacagtttagccaagaaattggtctggcaagcctgggtgcaagtgatgaagatgtgcagaaactggcaacctgttatttctttaccattgaatttggcctgtgcaaacaagagggtcagctgcgtgcatatggtgcaggtctgctgagcagcattggtgaactgaaacatgcactgagcgataaagcatgtgttaaagcatttgatccgaaaaccacctgtctgcaagaatgtctgattaccacctttcaagaagcctatttcgttagcgaaagctttgaagaggccaaagaaaaaatgcgcgattttgccaaaagcattacccgtccgtttagcgtttatttcaatccgtatacccagagcatcgagatcctgaaagatacctaa

SEQ ID NO: 180 - DNA, 1494 bp, Artificial
  Codon-optimized sequence
atgattagcaccgaaagcgatctgcgtcgtcagctggatgaaaatgttcgtagcgaagcagatgaaagcaccaaagaagaatgtccgtatattaacgcagttcagagccatcatcagaacgttcaagaaatgagcattattatcagcctggtgaaaaacatgaacgatatgaaaagcatcatcagcatctttaccgatcgcaacattaacatcctgcatattgaaagccgtctgggtcgtctgaatatgaaaaaacataccgaaaaaagcgaatttgagccgctggaactgctggttcatgttgaagttccgtgtattgaagttgaacgtctgctggaagaactgaaaagctttagcagctatcgtattgttcagaatccgctgatgaatctgccggaagcaaaaaatccgaccctggatgataaagtgccgtggtttccgcgtcatatttcagatctggataaagttagcaatagcgtgctgatgtatggcaaagaactggatgcagatcatccgggttttaaagataaagaatatcgcaaacgtcgcatgatgtttgcagatattgcactgaactataaatggggtcagcagattccgattgtggaatataccgaaattgaaaaaaccacctggggtcgtatttatcgtgaactgacccgtctgtataaaaccagcgcatgtcatgaatttcagaaaaatctgggtctgctgcaggataaagcaggctataatgaatttgatctgccgcagctgcaggttgttagcgatttcctgaaagcacgtaccggtttttgtctgcgtccggttgcaggttatctgagcgcacgtgattttctgagcggtctggcatttcgtgtgttttattgtacccagtatattcgtcatcaggccgatccgttttatactccggaaccggattgttgtcatgagctgctgggccatgttccgatgctggcagatccgaaatttgcacgttttagccaagaaattggtctggcaagcctgggcaccagtgatgaagaaatcaaaaaactggcaacctgctacttctttaccattgaatttggtctgtgccgtcaggataatcagctgaaagcatatggtgcaggtctgctgagcagcgttgcagaactgcagcatgcactgagcgataaagccgttattaaaccgtttattccgatgaaagtgatcaacgaagaatgcctggttaccacctttcagaatggttatttcgaaaccagcagctttgaagatgcaacccgtcagatgcgtgaatttgttcgtaccattaaacgtccgtttgatgtgcattataatccgtatacccagagcatcgaaattatcaaaaccccgaaaagcgttgccaaactggttcaggatctgcagtttgaactgacagcaattaatgaaagcctgctgaaaatgaacaaagaaattcgtagccagcagtttaccaccaacaaaattgttaccgaaaatcgcagcagctaa

SEQ ID NO: 181 - DNA, 735 bp, Artificial; identical to SEQ ID NO: 152
  Codon-optimized sequence

SEQ ID NO: 182 - DNA, 192 bp, Artificial
  Codon-optimized sequence
atggtcaagttgttcccatctgaaaatgctagaagatggcatagatggaaccacgaagttttgttgttggtcaacatccaatgctcattgaaacaaccattgtggtctgctgaaggtaaggttgataagaacagagaaaagtgtgctgccttcgtttacagattggttgaaattcaagatgccagaatttga

SEQ ID NO: 183 - DNA, 624 bp, Artificial
  Codon-optimized sequence
atgagcaccccgagcattcattgtctgaaaccgagtccgctgcatctgccgagcggtattccgggtagtccgggtcgtcagcgtcgccataccctgcctgcaaatgaatttcgttgtctgacaccggaagatgcagccggtgtttttgaaattgaacgtgaaccatttattagcgtgagcggtaattgtccgctgaacctggatgaagttcgtcattttctgaccctgtgtccggaactgagcctgggttggtttgttgaaggtcgtctggttgcatttatcattggtagcctgtgggatgaagaacgtctgacccaagaaagcctgaccctgcatcgtccgggtggtcgtaccgcacatctgcatgcactggcagttcatcattcatttcgtcagcagggcaaaggtagcgttctgctgtggcgttatctgcaacatgccggtggtcagcctgccgttcgtcgtgcagttctgatgtgtgaagatgcactggttccgttttatcagcgttttggttttcatccggcaggtccgtgtgcagttgttgtgggtagcctgacctttaccgaaatgcattgtagcctgcgtggtcatgcagcactgcgtcgtaatagcgatcgttaa

SEQ ID NO: 184 - DNA, 1038 bp, Artificial
  Codon-optimized sequence
atgggtagcagcgaagatcaggcatatcgtctgctgaatgattatgccaatggttttatggttagccaggttctgtttgcagcatgtgaactgggtgtttttgatctgctggcagaagcaccgggtccgctggatgttgcagcagttgcagccggtgttcgtgcaagcgcacatggcaccgaactgctgctggatatttgtgttagcctgaaactgctgaaagttgaaacccgtggtggtaaagcattttatcgtaataccgaactgagcagcgattatctgaccaccgttagcccgaccagccagtgtagcatgctgaaatatatgggtcgtaccagctatcgttgttggggtcatctggcagatgcagttcgtgaaggtcgtaatcagtatctggaaacctttggtgttccggcagaagaactgtttaccgcaatttatcgtagcgaaggtgaacgtctgcagtttatgcaggcactgcaagaggtttggagcgttaatggtcgtagcgttctgaccgcatttgatctgagcgtttttccgctgatgtgtgatctgggtggtggtgccggtgcactggcaaaagaatgtatgagcctgtatccgggttgtaaaattaccgtttttgatattccggaagttgtgtggaccgcaaaacagcattttagctttcaagaagaggaacaaatcgactttcaagagggcgattttttcaaagatccgctgccggaagcagatctgtatattctggcacgtgttctgcatgattgggcagatggtaaatgcagccatctgctggaacgtatttatcatacctgtaaaccgggtggtggcattctggttattgaaagcctgctggatgaagatcgtcgtggtccgctgctgacccagctgtatagcctgaatatgctggttcagaccgaaggtcaagaacgtaccccgacccattatcatatgctgctgagcagtgcaggttttcgtgattttcagttcaaaaaaaccggtgccatttatgatgcaatcctggcacgtaaataa

SEQ ID NO: 185 - protein, 428 aa, Homo sapiens
MDDKGNKGSSKREAATESGKTAVVFSLKNEVGGLVKALRLFQEKRVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLR

(19) United States
(12) Patent Application Publication
(10) Pub. No.: US 2016/0230200 A1
(43) Pub. Date: Aug. 11, 2016

(54) MICROORGANISMS FOR EFFICIENT PRODUCTION OF MELATONIN AND RELATED COMPOUNDS

(71) Applicant: DANMARKS TEKNISKE UNIVERSITET, KGS. Lyngby (DK)

(72) Inventors: JIANGFENG ZHU, NIVÅ (DK); NIELS BJERG JENSEN, COPENHAGEN S (DK); XIAO CHEN, VIRUM (DK); JOCHEN FÖRSTER, COPENHAGEN V (DK); IRINA BORODINA, NIVÅ (DK)

(21) Appl. No.: 14/916,205

(22) PCT Filed: Sep. 5, 2014
(86) PCT No.: PCT/EP2014/068967

§ 371 Date: Mar. 3, 2016

(30) Foreign Application Priority Data
Sep. 5, 2013 (EP) 13183120.8

(51) Int. Cl. C12P 17/10; C12N 9/88; C12N 9/06; C12N 9/12; C12N 9/04; C12N 9/78; C12N 9/02; C12N 9/10

(52) U.S. Cl. CPC C12P 17/10; C12N 9/0071; C12N 9/88; C12N 9/1029; C12N 9/1007; C12N 9/1085; C12N 9/1022; C12N 9/1294; C12N 9/0006; C12N 9/78; C12N 9/0028; C12N 9/1096; C12N 9/1205; C12Y 114/16004; C12Y 401/01028; C12Y 203/01087; C12Y 203/01005; C12Y 201/01004; C12Y 205/01054; C12Y 202/01001; C12Y 207/09002; C12Y 402/03012; C12Y 101/01153; C12Y 305/04016; C12Y 402/01096; C12Y 105/01034; C12Y 206/01057; C12Y 207/01001

(57) ABSTRACT

Recombinant microbial cells and methods for producing 5HTP, melatonin and related compounds using such cells are described. More specifically, the recombinant microbial cell may comprise exogenous genes encoding one or more of an L-tryptophan hydroxylase, a 5-hydroxy-L-tryptophan decarboxylyase, a serotonin acetyltransferase, an acetylserotonin O-methyltransferase; and means for providing tetrahydrobiopterin (THB), and can be further genetically modified to enrich one or more of tryptophan, S-adenosyl-L-methinonine and acetyl coenzyme A. Related sequences and vectors for use in preparing such recombinant microbial cells are also described.

### FIELD OF THE INVENTION

The present invention relates to recombinant microorganisms and methods for producing melatonin and related compounds, such as 5-hydroxytryptophan, serotonin and N-acetylserotonin. More specifically, the present invention relates to a recombinant microorganism comprising a heterologous gene encoding at least an L-tryptophan hydroxylase and means for providing tetrahydrobiopterin (THB). The invention also relates to methods of producing melatonin and related compounds using such microorganisms.

### BACKGROUND OF THE INVENTION

5-hydroxy-L-tryptophan (5HTP) is a naturally occurring amino acid and chemical precursor as well as metabolic intermediate in the biosynthesis of the neurotransmitters serotonin and melatonin from tryptophan. In animals and plants, 5HTP can be produced from the native metabolite L-tryptophan in one enzymatic step. In animals, the enzyme that catalyzes this reaction is tryptophan hydroxylase, which requires both oxygen and tetrahydropterin (THB, a.k.a. BH4) as cofactors. Specifically, tryptophan hydroxylase catalyzes the conversion of L-tryptophan (Schramek et al., 2001) and THB into 5-Hydroxy-L-tryptophan and 4a-hydroxytetra-hydrobiopterin (HTHB). 5HTP is then converted to serotonin by 5-hydroxy-L-tryptophan decarboxylyase. The conversion of serotonin to melatonin is catalyzed by serotonin acetyltransferase and acetylserotonin O-methyltransferase, with N-acetylserotonin as the metabolic intermediate.

Dietary supplements based on 5HTP for overcoming serotonin deficiency are available. Serotonin deficiency has been associated with a range of conditions, such as depression, obesity and insomnia. For such supplements, the primary source of 5HTP is typically seeds of Griffonia simplicifolia, but the extraction process can be costly and associated with low yields. Melatonin maintains the body's circadian rhythm and is a powerful anti-oxidant, and over-the-counter dietary supplements based on melatonin have been available for many years in the U.S. Typically, the melatonin is chemically synthesized. Thus, there is a need for a simplified and more cost-effective procedure.

U.S. Pat. No. 7,807,421 B2 and Yamamoto et al. (2003) describe cells transformed with enzymes participating in the biosynthesis of THB and a process for the production of a biopterin compound using the same.

WO 2012/135389 describes methods of producing oxidation products of an aromatic amino acid such as L-DOPA, 5HTP, serotonin and/or melatonin in a host cell which can, e.g., be capable of biosynthesizing BH4 (i.e., THB) or MH4 (tetrahydromonapterin) from GTP. BH4 biosynthesis in E. coli for the purpose of driving tyrosine hydroxylase-mediated tyrosine hydroxylation was not, however, successful, but endogenous MH4 was reportedly capable of replacing THB as cofactor for tyrosine hydroxylase.

### SUMMARY OF THE INVENTION

It has been found by the present inventors that 5HTP and melatonin, as well as related compounds such as, but not limited to, serotonin and N-acetylserotonin, can be produced in a recombinant microbial cell. Advantageously, these can be produced from an inexpensive carbon source, providing for a cost-efficient production.

Accordingly, the invention relates to a recombinant microbial cell comprising exogenous nucleic acid sequences encoding an L-tryptophan hydroxylase and enzymes providing at least one pathway for producing THB. Optionally, the microbial cell further comprises exogenous nucleic acid sequences encoding one, two or all of a 5-hydroxy-L-tryptophan decarboxylase, a serotonin acetyltransferase and an acetylserotonin O-methyltransferase. The microbial cell of the invention can further be genetically modified to provide for a more efficient production of the desired compound. Non-limiting examples include modifications in one or more endogenous metabolic pathways, thereby increasing or decreasing specific enzyme activities in the cell, and modifications increasing the supply of specific substrates or metabolites in such pathways, thereby improving the production of 5HTP, melatonin and related compounds. For example, in separate and specific embodiments, the microbial cell can comprise one or more genetic modifications providing for

an increase in the production of S-adenosyl-L-methinonine, the cofactor for acetylserotonin O-methyltransferase

an increase in acetyl coenzyme A production,

a decrease in 5HTP degradation,

an increase in tryptophan production, or

a combination of any two or more thereof.

The invention also relates to methods of producing 5HTP, melatonin or related compounds using such recombinant microbial cells, as well as for compositions comprising melatonin or a related compound produced by such recombinant microbial cells.

These and other aspects and embodiments are described in more detail in the following sections.

### LEGENDS TO THE FIGURES

FIG. 1 illustrates the metabolic pathways for the production of melatonin according to the invention.

FIG. 2 shows (A) 5-hydroxytryptophan production using the engineered S. cerevisiae strain ST783, and (B) comparative production of 5HTP in S. cerevisiae strains with and without the THB production and regeneration pathways (S. cerevisiae strain ST783 and 133-B8, respectively).

FIG. 3 shows melatonin production using S. cerevisiae ST892 strain.

FIG. 4 shows serotonin and N-acetylserotonin production from tryptophan using the S. cerevisiae ST925 strain.

FIG. 5 shows that the aro9 gene is partially responsible for the degradation of 5-hydroxytryptophan in S. cerevisiae.

FIG. 6 shows that tryptophanase is responsible for degrading 5-hydoxytryptophan into 5-hydroxyindole in the E. coli MG1655 wild type strain. (A): 5-hydroxytryptophan standard, (B): 5-hydroxyindole standard, (C): Culture of E. coli MG1655 tnaA-mutant strain, (D): Culture of E. coli MG1655 wild type strain.

FIG. 7 shows 5HTP production in E. coli strain MGT (pTHB, pTDP).

FIG. 8 shows detection of melatonin in SCE-iL3-HM-26 and SCE-iL3-27 by LC-MS compared to a standard. See Example 7 for details. (Both the Total Ion Chromatogram (TIC) and the Extracted Ion Chromatogram (XIC) are shown). (A) Standard (B) SCE-iL3-HM-26, (C) SCE-iL3-27.

### DETAILED DISCLOSURE OF THE INVENTION

The present invention provides for a recombinant microbial cell capable of efficiently producing 5HTP, melatonin or a related compound, including, but not limited to, serotonin or N-acetyl-serotonin, from an exogenously added carbon source.

In a first aspect, the invention relates to a recombinant microbial cell comprising exogenous nucleic acid sequences encoding

an L-tryptophan hydroxylase (TPH) (EC 1.14.16.4), a

5-hydroxy-L-tryptophan decarboxylase (DDC) (EC 4.1.1.28),

a serotonin acetyltransferase (AANAT) (EC 2.3.1.87 or EC 2.3.1.5),

an acetylserotonin O-methyltransferase (ASMT) (EC 2.1.1.4), and

one or more enzymes providing at least one exogenous pathway for producing THB.

Optionally, the microbial cell comprises a genetic modification providing for

(a) an increase in S-adenosyl-L-methinonine (SAM) production,
(b) an increase in acetyl coenzyme A (AcCoA) production,
(c) a decrease in 5-hydroxy-L-tryptophan (5HTP) degradation,
(d) an increase in tryptophan production, or
(e) a combination of any thereof, such as (a) and (b); (a) and (c); (a) and (d); (a), (b) and (c); (a), (b) and (d); (b) and (c); (b) and (d); (b), (c) and (d); and all of (a) to (d).

In this and other aspects and embodiments, an enzyme activity can be increased, e.g., by providing one or more exogenous nucleic acids and/or by upregulating the transcription or translation of one or more endogenous nucleic acids encoding enzymes having such an activity or activities.

In one embodiment, the microbial cell is genetically modified to increase the production of SAM. Genetic modifications increasing the production of SAM in the microbial cell include those increasing

(a) S-adenosylmethionine synthetase (EC 2.5.1.6) activity,
(b) ethionine resistance protein activity,
(c) S-adenosylhomocysteine hydrolase (EC 3.3.1.1) activity,
(d) methionine synthase (EC 2.1.1.-) activity, or
(e) a combination of any two or more of (a) to (d), such as (a) and (b); (a) and (c); (a) and (d); (b) and (c); (b) and (d); (c) and (d); (a), (b) and (c); (a), (b) and (d); (b), (c) and (d), and all of (a) to (d).

In one embodiment, the microbial cell of the preceding aspect or embodiment is genetically modified to provide for an increase in acetyl coenzyme A (AcCoA) production. Genetic modifications increasing the production of AcCoA in the microbial cell include those increasing

(a) AcCoA synthetase (EC 6.2.1.1) activity,
(b) acetylaldehyde dehydrogenase (EC 1.2.1.3) activity, and
(c) a combination of (a) and (b).

In a second aspect, the invention relates to a recombinant microbial cell comprising exogenous nucleic acid sequences encoding a TPH and one or more enzymes providing at least one exogenous pathway for producing THB.

Optionally, the microbial cell of this aspect is genetically modified to provide for a decrease in 5-hydroxy-L-tryptophan (5HTP) degradation, an increase in tryptophan production, or a combination thereof.

In one embodiment, the recombinant cell of any preceding aspect or embodiment is genetically modified to decrease 5HTP degradation. Genetic modifications decreasing 5HTP degradation include those decreasing

(a) aromatic amino acid aminotransferase (EC 2.6.1.57) activity,
(b) tryptophanase (EC 4.1.99.1) activity, and
(c) a combination of (a) and (b).

In this and other aspects and embodiments, decreasing an enzyme activity can be achieved, e.g., by deleting or downregulating one or more endogenous genes encoding enzymes having such an activity or activities. For example, as shown herein, in yeast cells such as Saccharomyces cerevisiae, aromatic amino acid transferase activity can be decreased by downregulating or deleting the aro9 gene or an ortholog thereof. In bacterial cells such as Escherichia coli cells, tryptophonase activity can be decreased by downregulating or deleting the tnaA gene or an ortholog thereof.

In one embodiment, the recombinant cell of any preceding aspect or embodiment is genetically modified to increase tryptophan production. Genetic modifications increasing tryptophan production include those

(a) decreasing tryptophan repressor transcription regulator activity,
(b) increasing 3-deoxy-d-heptulosonate-7-phosphate (DAHP) synthase (EC 2.5.1.54) activity;
(c) increasing transketolase (EC 2.2.1.1) and PEP synthase (EC 2.7.9.2) activity,
(d) decreasing the activity of one or more components of the phosphotransferase system (typically in bacteria),
(e) increasing hexokinase (EC 2.7.1.1) and, optionally, glucose facilitated diffusion protein (TC 2.A.1.1) activity, and
(f) a combination of any two or more of (a) to (e), such as (a) and (b); (a) and (c); (a) and (d); (a) and (e); (a), (b) and (c); (a), (b), and (d); (a), (b) and (e); (a), (c) and (d); (a), (c) and (e); (a), (d) and (e); (b) and (c); (b) and (d); (b) and (e); (c) and (d); (c) and (e); and (d) and (e), or more. In one embodiment, the recombinant cell comprises genetic modifications providing for (a), (b) and (c).

Pathways for producing THB include, but are not limited to, a pathway producing THB from guanosin triphosphate (GTP) and a pathway regenerating THB from 4a-hydroxytetrahydrobiopterin (HTHB). Accordingly, in one embodiment, the recombinant microbial cell of any preceding aspect or embodiment comprises exogenous nucleic acid sequences encoding a 6-pyruvoyl-tetrahydropterin synthase (PTPS) (EC 4.2.3.12), a sepiapterin reductase (SPR) (EC 1.1.1.153) and, optionally, a GTP cyclohydrolase I (GCH1) (EC 3.5.4.16). In one embodiment, the recombinant microbial cell of any preceding aspect or embodiment comprises exogenous nucleic acid sequences encoding a pterin-4-alpha-carbinolamine dehydratase (PCBD1) (EC 4.2.1.96); and, optionally, a dihydropteridine reductase (DHPR) (EC 1.5.1.34). In a preferred embodiment, the recombinant microbial cell of any preceding aspect or embodiment comprises exogenous nucleic acid sequences encoding a PTPS, an SPR, a GCH1, a PCBD1, and a DHPR, thereby providing exogenous enzymatic pathways producing THB from endogenous GTP and regenerating THB from HTHB.

In a third aspect, the invention relates to a method of producing melatonin, comprising culturing a recombinant microbial cell of the first aspect or any embodiment thereof in a medium comprising at least one carbon source and, optionally, isolating melatonin.

In one embodiment, the medium comprises at least 0.1 g/L methionine, at least 0.1 g/L SAM, or both. In another embodiment, the medium has not been supplemented with methionine, SAM, or any of methionine or SAM.

In a fourth aspect, the invention relates to a method of producing 5HTP, comprising culturing the recombinant microbial cell of the second aspect or any embodiment thereof in a medium comprising at least one carbon source and, optionally, isolating 5HTP.

In a fifth aspect, the invention relates to a method of producing a recombinant microbial cell, comprising transforming a microbial host cell with one or more vectors comprising nucleic acid sequences encoding a TPH, a DDC, an AANAT, an ASMT, a PTPS, an SPR, a GCH1, a PCBD1, and a DHPR, wherein the microbial cell is genetically modified to increase the production of at least one of SAM, AcCoA and tryptophan from a carbon source. In one embodiment, the microbial cell is genetically modified to increase SAM production. In one embodiment, the cell is genetically modified to increase AcCoA production. In one embodiment, the cell is genetically modified to increase tryptophan production. In one embodiment, the cell is genetically modified to increase SAM and AcCoA production. In one embodiment, the cell is genetically modified to increase SAM and tryptophan production. In one embodiment, the cell is genetically modified to increase AcCoA and tryptophan production. In one embodiment, the cell is genetically modified to increase SAM, AcCoA and tryptophan production. In any one of the preceding embodiments, the cell may further be genetically modified to decrease 5HTP degradation.

In a sixth aspect, the invention relates to a strain comprising recombinant microbial cells according to any preceding aspect or embodiment.

In a seventh aspect, the invention relates to a composition comprising melatonin obtainable by culturing a recombinant microbial cell according to any of the first and fifth aspects in a medium comprising a carbon source.

In an eighth aspect, the invention relates to a composition comprising 5HTP obtainable by culturing a recombinant microbial cell according to the second aspect in a medium comprising a carbon source.

In an ninth aspect, the present invention relates to a use of a composition comprising 5HTP or melatonin produced by a recombinant microbial cell or method described in any preceding aspect, in preparing a product such as, e.g., a dietary supplement, a pharmaceutical, a cosmeceutical, a nutraceutical, a feed ingredient or a food ingredient.

For any of the preceding aspects or embodiments, non-limiting examples of carbon sources include glucose, fructose, sucrose, xylose, mannose, galactose, rhamnose, arabinose, fatty acids, glycerol, acetate, starch, glycogen, amylopectin, amylose, cellulose, cellulose acetate, cellulose nitrate, hemicellulose, xylan, glucuronoxylan, arabinoxylan, glucomannan, xyloglucan, lignin, and lignocellulose. Preferably, the carbon source is glucose, xylose, or glycerol, or a mixture of any thereof. In one embodiment, the carbon source comprises, or consists essentially of, glucose. In one embodiment, the medium has not been supplemented with tryptophan, THB, or any of tryptophan and THB.

While the above aspects can be practiced in a microbial cell or strain of any suitable origin, such as bacterial, yeast, filamentous fungal, or algeal cells, for commercial applications, bacterial cells derived from Escherichia, Corynebacteria, Lactobaccillus, Bacillus or Pseudomonas cells, such as, e.g., E. coli, and yeast cells derived from Saccharomyces, Pichia or Yarrowia cells, such as e.g., S. cerevisiae, are particularly contemplated.

### DEFINITIONS

As used herein, “exogenous” means that the referenced item, such as a molecule, activity or pathway, is added to or introduced into the host cell or microorganism. For example, an exogenous molecule can be added to or introduced into the host cell or microorganism, e.g., via adding the molecule to the media in or on which the host cell or microorganism resides. An exogenous nucleic acid sequence can, for example, be introduced either as chromosomal genetic material by integration into a host chromosome or as non-chromosomal genetic material such as a plasmid. For such an exogenous nucleic acid, the source can be, for example, a homologous or heterologous coding nucleic acid that expresses a referenced enzyme activity following introduction into the host cell or organism. Similarly, when used in reference to a metabolic activity or pathway, the term refers to a metabolic activity or pathway that is introduced into the host cell or organism, where the source of the activity or pathway (or portions thereof) can be homologous or heterologous. Typically, an exogenous pathway comprises at least one heterologous enzyme.

As used herein, an “orthologous” gene of another gene is a gene inferred to be descended from the same ancestral sequence separated by a speciation event (i.e., when a species diverges into two separate species). Typically, orthologous genes encode proteins with a moderate to high sequence identity (e.g., at least about 15%, 20%, 30% or more) and/or can at least partially substitute for the other gene in terms of function, when transferred from one species into another. Orthologs of a particular gene can be identified using publicly available and specialized biological databases, e.g., by the eggNOG, InParanoid, OrthoDB, OrthoMCL, OMA, Roundup, TreeFam, LOFT, Ortholuge, EnsemblCompara GeneTrees and HomoloGene.

In the present context the term “heterologous” means that the referenced item, such as a molecule, activity or pathway, does not normally appear in the host cell or microorganism species in question.

As used herein, the terms “native” and “endogenous” means that the referenced item is normally present in or native to the host cell or microbal species in question.

As used herein, “upregulating” an endogenous gene means increasing the transcription and/or translation of a gene present in the native host cell genome relative to a control, such as e.g. the unmodified host cell. Methods of upregulating genes are known in the art and include, e.g., introducing a non-native promoter increasing transcription, modifying the native promoter, deleting genes encoding repressor protein, introducing multiple copies of the gene of interest, etc. “Downregulating” an endogenous gene as used herein means to reduce, optionally eliminate, the transcription or translation of an endogenous gene relative to a control, such as, e.g., the unmodified host cell. Methods of down-regulating, disrupting and deleting genes are known to those of skill in the art, and include, e.g., site-directed mutagenesis, genomic modifications based on homologous recombination, RNA degradation based on CAS9, etc.

As used herein, “vector” refers to any genetic element capable of serving as a vehicle of genetic transfer, expression, or replication for a exogenous nucleic acid sequence in a host cell. For example, a vector may be an artificial chromosome or a plasmid, and may be capable of stable integration into a host cell genome, or it may exist as an independent genetic element (e.g., episome, plasmid). A vector may exist as a single nucleic acid sequence or as two or more separate nucleic acid sequences. Vectors may be single copy vectors or multicopy vectors when present in a host cell. Preferred vectors for use in the present invention are expression vector molecules in which one or more functional genes can be inserted into the vector molecule, in proper orientation and proximity to expression control elements resident in the expression vector molecule so as to direct expression of one or more proteins when the vector molecule resides in an appropriate host cell.

The term “host cell” or “microbial” host cell refers to any microbial cell into which an exogenous nucleic acid sequence can be introduced and expressed, typically via an expression vector. The host cell may, for example, be a wild-type cell isolated from its natural environment, a mutant cell identified by screening, a cell of a commercially available strain, or a genetically engineered cell or mutant cell, comprising one or more other exogenous and/or heterologous nucleic acids than those of the invention.

A “recombinant cell” or “recombinant microbial cell” as used herein refers to a host cell into which one or more exogenous nucleic acid sequences of the invention have been introduced, typically via transformation of a host cell with a vector.

Unless otherwise stated, the term “sequence identity” for amino acid sequences as used herein refers to the sequence identity calculated as (n_{ref}−n_{dif})·100/n_{ref}, wherein n_{dif} is the total number of non-identical residues in the two sequences when aligned and wherein n_{ref} is the number of residues in one of the sequences. Hence, the amino acid sequence GSTDYTQNWA will have a sequence identity of 80% with the sequence GSTGYTQAWA (n_{dif}=2 and n_{ref}=10). The sequence identity can be determined by conventional methods, e.g., Smith and Waterman, (1981), Adv. Appl. Math. 2:482, by the ‘search for similarity’ method of Pearson & Lipman, (1988), Proc. Natl. Acad. Sci. USA 85:2444, using the CLUSTAL W algorithm of Thompson et al., (1994), Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., (1990), Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

Enzymes referred to herein can be classified on the basis of the handbook Enzyme Nomenclature from NC-IUBMB, 1992), see also the ENZYME site at the internet: http://www.expasy.ch/enzyme/. This is a repository of information relative to the nomenclature of enzymes, and is primarily based on the recommendations of the Nomenclature Committee of the International Union of Biochemistry and Molecular Biology (IUB-MB). It describes each type of characterized enzyme for which an EC (Enzyme Commission) number has been provided (Bairoch A. The ENZYME database, 2000, Nucleic Acids Res 28:304-305). The IUBMB Enzyme nomenclature is based on the substrate specificity and occasionally on their molecular mechanism.

In the present disclosure, tryptophan is of L-configuration, unless otherwise noted.

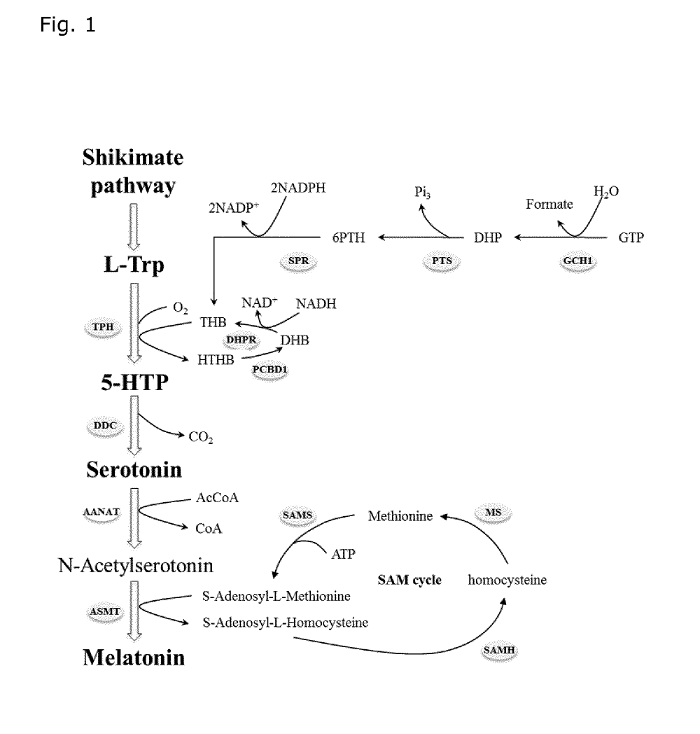

The term “substrate”, as used herein in relation to a specific enzyme, refers to a molecule upon which the enzyme acts to form a product. When used in relation to an exogenous biometabolic pathway, the term “substrate” refers to the molecule upon which the first enzyme of the referenced pathway acts, such as, e.g., GTP in the pathway shown in FIG. 1 which produces THB from GTP (see FIG. 1). When referring to an enzyme-catalyzed reaction in a microbial cell, an “endogenous” substrate or precursor is a molecule which is native to or biosynthesized by the microbial cell, whereas an “exogenous” substrate or precursor is a molecule which is added to the microbial cell, via a medium or the like.

Construction of appropriate expression vectors and other recombinant or genetic modification techniques for practising the invention are well known in the art (see, e.g., Green and Sambrook, Molecular Cloning, A Laboratory Manual, Cold Spring Harbor Laboratory Press (Cold Spring Harbor, N.Y.) (2012), and Ausubel et al., Short Protocols in Molecular Biology, Current Protocols John Wiley and Sons (New Jersey) (2002), and references cited herein). Appropriate microbial cells and vectors are available commercially through, for example, the American Type Culture Collection (ATCC), Rockville, Md.

The following are abbreviations and the corresponding EC numbers for enzymes referred to herein and in the Figures.

| Abbreviation | Enzyme | EC# |
|---|---|---|
| GCH1 | GTP cyclohydrolase I | EC 3.5.4.16 |
| PTPS | 6-pyruvoyl-tetrahydropterin synthase | EC 4.2.3.12 |
| SPR | sepiapterin reductase | EC 1.1.1.153 |
| DHPR | dihydropteridine reductase | EC 1.5.1.34 |
| PCBD1 | pterin-4-alpha-carbinolamine dehydratase, | EC 4.2.1.96 |
| | a.k.a. 4a-hydroxytetrahydrobiopterin | |
| | dehydratase | |
| TPH2 | L-tryptophan hydroxylase 2 | EC 1.14.16.4 |
| TPH1 | L-tryptophan hydroxylase 1 | EC 1.14.16.4 |
| T5H | tryptamine 5-hydroxylase | EC 1.14.16.4 |
| TDC | L-Tryptophan decarboxy-lyase | EC 4.1.1.28 |
| DDC | 5-Hydroxy-L-tryptophan decarboxylase, | EC 4.1.1.28 |
| | a.k.a., 5-hydroxy-L-tryptophan | |
| | decarboxy-lyase | |
| AANAT | serotonin acetyltransferase | EC 2.3.1.87 or |
| | | EC 2.3.1.5 |
| ASMT | acetylserotonin O-methyltransferase | EC 2.1.1.4 |
| SAMS | S-adenosylmethionine synthetase | EC 2.5.1.6 |
| SAMH | S-adenosylhomocysteine hydrolase | EC 3.3.1.1 |
| MS | Methionine synthase | EC 2.1.1.— |
| ACS | AcCoA synthetase | EC 6.2.1.1 |
| ADH | Acetylaldehyde dehydrogenase | EC 1.2.1.3 |
| | Aromatic amino acid aminotransferase | EC 2.6.1.57 |
| | Tryptophanase | EC 4.1.99.1 |
| | 3-Deoxy-d-heptulosonate-7-phosphate | EC 2.5.1.54 |
| | (DAHP) synthase | |
| | Transketolase | EC 2.2.1.1 |
| PPS | Phosphoenolpyruvate (PEP) synthase | EC 2.7.9.2 |
| | Hexokinase | EC 2.7.1.1 |
| | Glucose facilitated diffusion protein | TC 2.A.1.1 |

The following are abbreviations and the corresponding PubChem numbers for metabolites referred to herein and in the Figures.

| Metabolite | | |
|---|---|---|
| Abbreviation | Metabolite | PubChem CID |
| GTP | guanosine triphosphate | 6830 |
| DHP | 7,8-dihydroneopterin 3′-triphosphate | 121885 |
| 6PTH | 6-pyruvoyltetrahydropterin | 128973 |
| THB | Tetrahydrobiopterin | 1125 |
| HTHB | 4a-hydroxytetrahydrobiopterin | 129803 |
| DHB | Dihydrobiopterin | 119055 |
| SAM | S-adenosyl-L-methionine | 34755 |
| SAH | S-adenosyl-L-homocysteine | 439155 |
| PEP | Phosphoenolpyruvate | 1005 |
| E4P | Erythrose 4-phosphate | 122357 |
| DHAP | 3-Deoxy-d-heptulosonate-7-phosphate | 5460215 |

### Specific Embodiments of the Invention

As shown in the present Examples, 5HTP, melatonin and related compounds such as serotonin and N-acetylserotonin, can be produced in a microbial cell transformed with enzymes of a THB-dependent pathway, outlined in FIG. 1.

The first reaction (TPH) in this pathway converts tryptophan into 5HTP. The reaction requires a metabolic cofactor, tetrahydrobiopterin (THB), which is not natively present in wild-type microbial cells such as wild-type Saccharomyces cerevisiae or Escherichia coli. In order to produce 5HTP and melatonin in such cells, two exogenous pathways for the synthesis and regeneration of the THB cofactor, respectively, should therefore be introduced to avoid having to add chemically synthesized THB.

The pathway for THB synthesis comprises a GCH1, a PTS, and a SPR and converts GTP into THB (Yamamoto et al., 2003). Among these three enzymes, the GCH1 is natively present in some wild-type microbial cells, e.g., S. cerevisiae and E. coli, while the PTS and SPR must be introduced in order to generate THB in these microorganisms.

The pathway for THB regeneration comprises a PCBD1 and a DHPR. These two enzymes reduce the 4-alpha-hydroxy-tetrahydrobiopterin (HTHB) produced through the tryptophan hydroxylase reaction into THB. E. coli has a native protein (NfsB) functioning as a DHPR (Crabtree and Channon, 2011).

As shown in Examples 1 and 2, S. cerevisiae and E. coli cells in which these exogenous pathways have been constructed are capable of efficiently producing 5HTP from a carbon source and tryptophan substrate but without adding THB cofactor.

Accordingly, in one embodiment, the invention provides a recombinant microbial cell comprising exogenous nucleic acid sequences encoding a TPH (EC 1.14.16.4) and enzymes providing at least one pathway for producing THB. In one preferred embodiment, the TPH is a Homo sapiens THP2 or a Schistosoma mansoni TPH, or a functionally active variant or fragment of any thereof, as described in more detail below, In a preferred embodiment, the microbial cell further comprises a genetic modification providing for an increase in tryptophan production, a decrease in 5HTP degradation, or a combination of both.

The production of melatonin from tryptophan requires four enzymatic reactions, namely TPH, DDC, AANAT, and ASMT. In addition to THB, the melatonin pathway also requires acetyl coenzyme A (AcCoA) for the AANAT reaction, and S-adenosyl-methionine (SAM) for the ASMT reaction, respectively. AcCoA serves as a metabolic cofactor in the AANAT reaction, but is also part of other, endogenous pathways in microbial cells. SAM is a principal methyl donor in various intracellular transmethylation reactions. It is synthesized in the cell through SAM synthetase from methionine and ATP, and natively generated through the SAM cycle, which consists of a methyl transferase, an S-adenosyl-L-homocysteine hydrolase, a folate transferase, and an S-adenosyl-methionine synthetase (Lee et al., 2010).

As shown in Example 1, S. cerevisiae cells in which the required exogenous enzymes have been recombinantly introduced to form these pathways are capable of efficiently producing 5HTP from a carbon source and tryptophan substrate but without adding THB cofactor.

Accordingly, in one embodiment, the invention provides a recombinant microbial cell comprising exogenous nucleic acid sequences encoding a TPH, a DDC, an ASMT and enzymes providing at least one pathway for producing THB.

In a preferred embodiment, the microbial cell comprises a genetic modification providing for an increase in S-adenosyl-L-methinonine (SAM) production, an increase in acetyl coenzyme A (AcCoA) production, an increase in tryptophan production, a decrease in 5HTP degradation, or a combination of any thereof. These are further described below.

In the present context, “overexpressing” refers to introducing an exogenous nucleic acid encoding an enzyme which is either heterologous or native to the microbial host cell, or is a functionally active fragment or variant thereof, and expressing the exogenous nucleic acid to increase the enzyme activity in the microbial cell as compared to the microbial host cell without the introduced exogenous nucleic acid, e.g., a native microbial host cell. In case of a microbial host cell which does not normally contain the enzymatic activity referred to, or where the native enzymatic activity is insufficient, or the native enzyme is subjected to unwanted regulation, an exogenous nucleic acid encoding an enzyme which is heterologous to the microbial host cell and which has the desired activity and regulation patterns can be introduced. Overexpression of an exogenous, e.g., a heterologous, nucleic acid can be achieved by placing the nucleic acid under the control of a strong promoter. Non-limiting examples of strong promoters suitable for, e.g., yeast cells are TEF1, PGK1, HXT7 and TDH3.

(1) Tryptophan Enrichment:

Tryptophan is the precursor of the 5HTP and melatonin production pathways according to the current inventions. Tryptophan can optionally be supplemented into the culture medium, and another carbon source such as glucose or glycerol are added in order to generate energy or metabolic cofactors for the 5HTP or melatonin pathways. Avoiding tryptophan supplementation would be advantageous, however, reducing the cost of the process as well as unwanted physiological effects of tryptophan on the cell such as feedback inhibitions.

Thus, in one embodiment, in order to produce 5HTP or melatonin from simple carbon sources such as glucose, fructose, xylose, glycerol, and others mentioned below, the recombinant microbial cell is genetically modified to enrich the generation of tryptophan. This can be achieved either by releasing feedback inhibitions in the shikimate pathway, enriching precursors for the shikimate pathway, or a combination of both. Both strategies can be implemented in several different ways, outlined below. See also Example 5 for further details.

Releasing Feedback Inhibitions in the Shikimate Pathway:

(a) In one embodiment, an endogenous gene encoding a tryptophan repressor transcription regulator in the microbial cell is deleted or downregulated. For example, in E. coli, the trpR gene encodes a tryptophan transcriptional regulator protein, which forms a complex with tryptophan molecule to negatively regulate the expression of trpABCDE genes. Deactivating TrpR by knocking out the trpR gene (ΔtrpR) improves tryptophan production in the cell. In other microbial cells, orthologous genes to E. coli trpR such as TrpR (Klebsiella pneumonia), TrpR (Mannheimia succiniciproducens), and BirA (Bacillus subtilis) can likewise be downregulated or deleted to improve tryptophan production.

(b) In one embodiment, the microbial cell is genetically modified to overexpress a feedback resistant 3-deoxy-d-heptulosonate-7-phosphate (DAHP) synthase (EC 2.5.1.54). The DAHP synthase is a key enzyme in the pathway for aromatic amino acid synthesis in E. coli, and is subject to feedback inhibition by tryptophan, tyrosine, and phenylalanine. Overexpressing a feedback resistant version of DAHP synthase such as AroG* in E. coli, corresponding to ARO4 in S. cerevisiae improves tryptophan production in the cell. Exemplary DAHP synthases for overexpression include those listed in Table 1, as well as functionally active variants, homologs and fragments thereof.

### Enriching Precursors for Shikimate Synthesis:

Phosphoenolpyruvate (PEP) and erythrose 4-phosphate (E4P) are the two major precursors for the synthesis of aromatic amino acids. In order to efficiently produce tryptophan from simple carbon sources, the cell is engineered to enrich intracellular PEP and E4P.

(a) In one embodiment, the microbial cell is genetically modified to overexpress transketolase (EC 2.2.1.1) and PEP synthase (EC 2.7.9.2). This enriches PEP and E4P concentrations in the microbial cells and thereby also tryptophan. Exemplary transketolases and PEP synthases include those shown in Table 1, as well as functionally active variants, homologs and fragments thereof.

(b) In one embodiment, the microbial cell comprises a deletion or downregulation of endogenous genes encoding one or more components of the phosphotransferase system (PTS), thereby disrupting it. The PTS is a glucose uptake system in bacteria using PEP as the energy source. For example, in E. coli, the PTS can be disrupted by introducing a point-mutation in or knocking out one or more of the genes in the ptsHIcrr operon, which encodes the Enzyme I (EI), Histidine protein (HPr), and Enzyme II (EII) (Meadow et al., 1990). Optionally, this can be made i combination with overexpressing a hexokinase (EC 2.7.1.1) and a glucose facilitated diffusion protein (TC 2.A.1.1). Exemplary hexokinases (e.g., ATP dependent hexokinase) and glucose facilitated diffusion proteins (e.g., phosphoenolpyruvate-dependent glucose transporter) include those shown in Table 1, as well as functionally active variants, homologs and fragments thereof.

(2) SAM Enrichment

SAM is an essential metabolic cofactor required for the ASMT reaction to convert N-acetylserotonin into melatonin. Enriching SAM in the cell shifts the reaction equilibrium towards the generation of melatonin, thereby increasing the production of melatonin by the recombinant melatonin-producing microbial cells described herein. This can be achieved according to one or more of the following:

(a) In one embodiment, the recombinant microbial cell is genetically modified to comprise an exogenous nucleic acid sequence (e.g. an artificial operon) encoding SAM synthetase (EC 2.5.1.6) for improving the supply of SAM. Exemplary SAM synthetases for this embodiment include those shown in Table 1, as well as functionally active variants, homologs and fragments thereof. In S. cerevisiae, two isozymes exist; SAM1 and SAM2. In one embodiment, the microbial cell is an S. cerevisiae cell genetically modified to comprise an exogenous sequence encoding SAM2. Optionally, for melatonin production, the culture medium of the recombinant microbial cell is supplemented with methionine, e.g., at about 0.01 to about 10 g/L, such as about 0.02 to about 5 g/L, such as about 0.03 to about 3 g/L, such as about 0.1 to 1 g/L, or at least about 0.05 g/L, or at least about 0.1 g/L.

(b) In one embodiment, the recombinant microbial cell overexpresses SAM synthetase with the ethionine resistance gene ERC1 product. Exemplary SAM synthetases and ethionine resistance proteins include those listed in Table 1, as well as functionally active variants, homologs and fragments thereof.

(c) In one embodiment, the recombinant microbial cell overexpresses SAM cycle enzymes. For example, the recombinant microbial can comprise overexpressed SAM synthetase (EC 2.5.1.6), S-adenosylhomocysteine hydrolase (EC 3.3.1.1), and methionine synthases (EC 2.1.1.-) for the continuous supply of SAM for melatonin production. Exemplary SAM synthetases, S-adenosylhomocysteine hydrolases and methionine synthases include those listed in Table 1, as well as functionally active variants, homologs and fragments thereof.

(d) In one embodiment, the culture medium of the recombinant microbial cell is directly supplemented with SAM, e.g., at about 0.01 to about 1.5 g/L, such as about 0.1 to about 1 g/L, or at least about 0.05 g/L, or at least about 0.1 g/L. Optionally, the culture medium is also supplemented with methionine, as described above.

(3) Enriching AcCoA Supply

Acetyl coenzyme A (AcCoA) is a cofactor used by the AANAT reaction. Enriching AcCoA in the cell, e.g., by feeding carbon sources such as glucose during the cell culture, can improve AcCoA availability and thereby improve the production of melatonin. Endogenous pathways compete for use of AcCoA as a substrate, however, but it has been shown that engineering the metabolic pathways can improve the supply of AcCoA (Kocharin et al., 2012). Therefore, to promote the ASMT reaction, a cytosolic acetaldehyde dehydrogenase (EC 1.2.1.4) and an acetyl coenzyme A synthetase (EC 6.2.1.1) can be introduced in order to convert acetaldehyde into AcCoA.

Accordingly, in one embodiment, the recombinant microbial cell comprises one or more exogenous nucleic acid sequences encoding an AcCoA synthetase (EC 6.2.1.1), an acetylaldehyde dehydrogenase (EC 1.2.1.3), or a combination thereof. Exemplary AcCoA synthetases and acetylaldehyde dehydrogenases include those shown in Table 1, as well as functionally active variants, homologs and fragments thereof.

(4) Decreasing 5HTP Degradation.

In one embodiment, the recombinant microbial cell of the invention comprises a deletion or downregulation of an endogenous gene encoding an aromatic amino acid aminotransferase (EC 2.6.1.57), and/or a deletion or downregulation of an endogenous gene encoding a tryptophanase (EC 4.1.99.1).

In one embodiment, the microbial cell is genetically modified, typically mutated, to downregulate or delete tryptophanase activity. Tryptophanase or tryptophan indole-lyase (EC 4.1.99.1), encoded by the tnaA gene in E. coli, catalyzes the hydrolytic cleavage of L-tryptophan to indole, pyruvate and NH4+. Active tryptophanase consists of four identical subunits, and enables utilization of L-tryptophan as sole source of nitrogen or carbon for growth together with a tryptophan transporter encoded by tnaC gene. Tryptophanase is a major contributor towards the cellular L-cysteine desulfhydrase (CD) activity. In vitro, tryptophanase also catalyzes a, β elimination, β replacement, and a hydrogen exchange reactions with a variety of L-amino acids (Watanabe, 1977). As shown in Example 2, E. coli tryptophanase can degrade also 5HTP, thus reducing the yield of 5HTP.

Tryptophan degradation mechanisms are known to also exist in other microorganisms. For instance, in S. cerevisiae, there are two different pathways for the degradation of tryptophan (The Erlich pathway and the kynurenine pathway, respectively), involving in their first step the aromatic amino acid aminotransferase ARO8, ARO9, ARO10, and/or BNA2 genes. Reducing tryptophan degradation, such as by reducing tryptophanase activity, can be achieved by, e.g., a site-directed mutation in or deletion of a gene encoding a tryptophanase, such as the tnaA gene (in E. coli or other organisms such as Enterobacter aerogenes) (Uniprot P0A853), or the kynA gene (in Bacillus species) (Uniprot Q736W5), or one or more of the ARO8 (Uniprot P53090), ARO9 (Uniprot P38840), ARO10 (Uniprot Q06408) and BNA2 (Uniprot P47125) genes (in S. cerevisiae). In one embodiment, the ARO9 gene is downregulated, optionally deleted. Alternatively, tryptophanase activity can be reduced reducing the expression of the gene by introducing a mutation in, e.g., a native promoter element, or by adding an inhibitor of the tryptophanase.

### Combinations of Genetic Modifications According to (1) to (4)

Various combinations of the above-mentioned genetic modifications are also contemplated. For example, in one embodiment, the recombinant microbial cell is a cell where a feedback-resistant DAHP synthase, transketolase, and phosphoenolpyruvate synthase are overexpressed. In another embodiment, the recombinant microbial cell is a cell where SAM synthetase, ethionine resistance gene ERC1, feedback resistant DAHP synthase, transketolase, and phosphoenolpyruvate synthase are overexpressed. In another embodiment, the recombinant microbial cell is a cell where SAM synthetase, acetaldehyde dehydrogenase, and AcCoA synthetase are overexpressed. In one preferred embodiment, the microbial cell is an S. cerevisiae cell. In one preferred embodiment, the microbial cell is an E. coli cell.

### Tryptophan Hydroxylase (TPH)

Sources of nucleic acid sequences encoding an L-tryptophan hydroxylase include any species where the encoded gene product is capable of catalyzing the referenced reaction, including humans, mammals such as, e.g., mouse, cow, horse, chicken and pig, as well as other animals such as, e.g., the parasite Schistosoma mansoni. In humans and, it is believed, in other mammals, there are two distinct TPH alleles, referred to herein as TPH1 and TPH2, respectively.

Exemplary nucleic acids encoding L-tryptophan hydroxylase for use in aspects and embodiments of the present invention include, but are not limited to, those encoding the TPHs listed in Table 1, as well as functionally active variants, homologs and fragments thereof. Functional fragments of TPH enzymes are known in the art. For example, to increase heterologous expression in E. coli and the enzyme stability, SEQ ID NO:1 can be double truncated to remove the regulatory and interface domains of the full-length enzyme (SEQ ID NO:1) so that only the catalytic core of the enzyme remains, corresponding to amino acids Met102 to Ser416, (Moran, Daubner, & Fitzpatrick, 1998). Other TPH sequences can be similarly truncated to create functionally active fragments comprising the catalytic core, optionally comprising the segment corresponding to Met102 to Ser416 of any one of SEQ ID NOS:2 to 8 or a variant or homolog thereof, when aligned with SEQ ID NO:1. For example, SEQ ID NOS:9, 175 and 176 all represent truncated versions of Homo sapiens TPH2 (SEQ ID NO:3), although SEQ ID NOS:9 and 175 further comprise heterologous 20-amino acid polypeptides at their C-terminal. In a preferred embodiment of any aspect of the invention, the TPH comprises or consists essentially of a truncated Homo sapiens TPH2 or Schistosoma mansoni TPH, the latter having advantageous properties with respect to, e.g., solubility and thus enabling no or less truncation of the enzyme sequence. In one embodiment of any aspect of the invention, the TPH comprises or consists essentially of SEQ ID NO:176. In one embodiment of any aspect of the invention, the TPH comprises or consists essentially of SEQ ID NO:177.

Assays for measuring L-tryptophan hydroxylase activity in vitro are well-known in the art (see, e.g., Winge et al. (2008), Biochem. J., 410, 195-204 and Moran, Daubner, & Fitzpatrick, 1998) and described in the present Examples. In the recombinant host cell, the L-tryptophan hydroxylase is typically sufficiently expressed so that an increased level of 5HTP production from L-tryptophan can be detected as compared to the microbial host cell prior to transformation with the TPH, optionally in the presence of added THB cofactor and/or tryptophan substrate.

### THB Pathways

In one embodiment, the recombinant cell comprises an exogenous pathway producing THB from GTP and herein referred to as “first THB pathway”, comprising a GTP cyclohydrolase I (GCH1), a 6-pyruvoyl-tetrahydropterin synthase (PTPS), and a sepiapterin reductase (SPR) (see FIG. 1). The addition of such a pathway to microbial cells such as E. coli (JM101 strain), S. cerevisiae (KA31 strain) and Bacillus subtilis (1A1 strain (TrpC2)) has been described, see, e.g., Yamamoto (2003) and U.S. Pat. No. 7,807,421, which are hereby incorporated by reference in their entireties.

In the recombinant host cell, the enzymes of the first THB pathway are typically sufficiently expressed in sufficient amounts to detect an increased level of 5HTP production from L-tryptophan as compared to the recombinant microbial cell without transformation with these enzymes (i.e., the recombinant cell comprising only L-tryptophan hydroxylase), or to another suitable control. Exemplary assays for measuring the level of 5HTP production from L-tryptophan is provided in Examples 1 and 2. Alternatively, the expression and activity of the enzymes of the first THB pathway, i.e., production of THB or related products, can be tested according to methods described in Yamamoto (2003), U.S. Pat. No. 7,807,421, or Woo et al. (2002), Appl. Environ. Microbiol. 68, 3138, or other methods known in the art.

The GCH1 is typically classified as EC 3.5.4.16, and converts GTP to DHP in the presence of its cofactor, water, as shown in FIG. 1. Exemplary nucleic acids encoding GCH1 enzymes for use in aspects and embodiments of the present invention include, but are not limited to, those encoding the GCH1s listed in Table 1, as well as functionally active variants, homologs and fragments thereof.

In some embodiments, the microbial host cell endogenously comprises sufficient amounts of a native GCH1. In these cases transformation of the host cell with an exogenous nucleic acid encoding a GCH1 is optional. In other embodiments, the exogenous nucleic acid encoding a GCH1 can encode a GCH1 which is endogenous to the microbial host cell, e.g., in the case of host cells such as E. coli, S. cerevisiae, Bacillus subtilis and Streptomyces avermitilis. In E. coli, for example, the expression of the GCH1 gene is regulated by the SoxS system. Should higher levels of GCH1 be needed, GCH1 from E. coli or another suitable source can be provided exogenously.

The PTPS is typically classified as EC 4.2.3.12, and converts DHP to 6PTH, as shown in FIG. 1. Sources of nucleic acid sequences encoding a PTPS include any species where the encoded gene product is capable of catalyzing the referenced reaction, including human, mammalian and microbial species. Non-limiting and exemplary nucleic acids encoding PTPS enzymes for use in aspects and embodiments of the present invention include those encoding the PTPSs shown in Table 1, as well as functionally active variants, homologs and fragments thereof.

In some embodiments, the microbial host cell endogenously comprises a sufficient amount of a native PTPS. In these cases transformation of the host cell with an exogenous nucleic acid encoding a PTPS is optional. In other embodiments, the exogenous nucleic acid encoding a PTPS can encode a PTPS which is endogenous to the microbial host cell, e.g., in the case of host cells such as Streptococcus thermophilus.

The SPR is typically classified as EC 1.1.1.153, and converts 6PTH to THB in the presence of its cofactor NADPH, as shown in FIG. 1. Exemplary nucleic acids encoding SPR enzymes for use in aspects and embodiments of the present invention include, but are not limited to, those encoding the SPRs shown in Table 1, as well as functionally active variants, homologs and fragments thereof.

In one embodiment, the recombinant cell comprises a pathway producing THB by regenerating THB from HTHB, herein referred to as “second THB pathway”, comprising a 4a-hydroxytetrahydrobiopterin dehydratase (PCBD1) and a 6-pyruvoyl-tetrahydropterin synthase (DHPR). As shown in FIG. 1, the second THB pathway converts the HTHB formed by the L-tryptophan hydroxylase-catalyzed hydroxylation of L-tryptophan back to THB, thus allowing for a more cost-efficient 5HTP production.

In the recombinant host cell, the enzymes of the second THB pathway are typically sufficiently expressed so that an increased level of 5HTP production from L-tryptophan can be detected as compared to the recombinant microbial cell without transformation with these enzymes (i.e., the recombinant cell comprising only L-tryptophan hydroxylase) in the presence of a THB source, or to another suitable control.

The PCBD1 is typically classified as EC 4.2.1.96, and converts HTHB to DHB in the presence of water, as shown in FIG. 1. Exemplary nucleic acids encoding GCH1 enzymes for use in aspects and embodiments of the present invention include, but are not limited to, those encoding the PCBD1s shown in Table 1, as well as functionally active variants, homologs and fragments thereof. In some embodiments, the microbial host cell endogenously comprises a sufficient amount of a native PCBD1. In these cases, transformation of the host cell with an exogenous nucleic acid encoding a PCBD1 is optional. In other embodiments, the exogenous nucleic acid encoding a PCBD1 can encode a PCBD1 which is endogenous to the microbial host cell, e.g., in the case of host cells from Bacillus cereus, Corynebacterium genitalium, Lactobacillus ruminis or Rhodobacteraceae bacterium.

The DHPR is typically classified as EC 1.5.1.34, and converts DHB to THB in the presence of cofactor NADH, as shown in FIG. 1. Exemplary nucleic acids encoding DHPR enzymes for use in aspects and embodiments of the present invention include, but are not limited to, those encoding DHPRs shown in Table 1, as well as functionally active variants, homologs or catalytically active fragments thereof.

### Combination of First and Second THB Pathway

As shown in FIG. 1, a successful combination of both the first and second THB pathways in the recombinant cell, introducing pathways for producing THB from GTP and for regenerating THB consumed by L-tryptophan hydroxylase, is especially advantageous, since the addition of THB, as well as the addition of L-tryptophan, can be avoided, allowing for 5HTP production from an inexpensive carbon source. As shown in Example 5, 5HTP production was obtained in a recombinant E. coli strain (comprising both the first and second THB pathways) in LB medium supplemented with glucose and/or L-tryptophan. In M9 medium, supplementation with tryptophan produced the highest 5HTP measurements. Accordingly, in one embodiment, the invention provides for recombinant microbial cells, processes and methods where the recombinant host cell comprises both the first and second THB pathways of any preceding aspect or embodiment.

### 5-Hydroxy-L-Tryptophan Decarboxy-Lyase

The last step in the serotonin biosynthesis via a 5HTP intermediate, the conversion of 5HTP to serotonin, is in animal cells catalyzed by a 5-hydroxy-L-tryptophan decarboxy-lyase (DDC), which is an aromatic L-amino acid decarboxylase typically classified as EC 4.1.1.28. See FIG. 1. Suitable DDCs include any tryptophan decarboxylase (TDC) capable of catalyzing the referenced reaction. TDC participates in the plant serotonin biosynthesis pathway, where tryptophan decarboxylase (TDC) catalyzes the conversion of tryptophan to tryptamine, which is then converted into serotonin in a reaction catalyzed by tryptamine 5-hydroxylase (T5H). TDC likewise belongs to the aromatic amino acid decarboxylases categorized in EC 4.1.1.28, and can be able to convert 5HTP to serotonin and carbon dioxide (see, e.g., Park et al., 2008, and Gibson et al., J. Exp. Bot. 1972; 23(3):775-786), and thus function as a DDC. Exemplary nucleic acids encoding DDC enzymes for use in aspects and embodiments of the present invention include, but are not limited to, those encoding the DDCs listed in Table 1, as well as functionally active variants, homologs and fragments thereof. In some embodiments, particularly where it is desired to also promote serotonin formation from a tryptamine substrate in the same recombinant cell, an enzyme capable of catalyzing both the conversion of tryptophan to tryptamine and the conversion of 5HTP to serotonin can be used. For example, rice TDC and tomato TDC can function also as a DDC, an activity which can be promoted by the presence of pyridoxal phosphate (e.g., at a concentration of about 0.1 mM) (Park et al., 2008; and Gibson et al., 1972).

Suitable assays for testing serotonin production by a DDC in a recombinant microbial host cell are provided in, or can be adapted from, e.g., Park et al. (2008) and (2011). For example, these assays can be adapted to test serotonin production by a TDC or DDC, either from 5HTP or, in case the microbial cell comprises an L-tryptophan hydroxylase, from L-tryptophan (or simply a carbon source). In one exemplary embodiment, the recombinant microbial cell produces at least 5%, such as at least 10%, such as at least 20%, such as at least 50%, such as at least 100% or more serotonin than the recombinant cell without transformation with DDC/TDC enzymes, i.e., a background value.

### Serotonin Acetyltransferase

In one aspect, the recombinant microbial cell further comprises an exogenous nucleic acid sequence encoding a serotonin acetyltransferase, also known as serotonin-N-acetyltransferase, arylalkylamine N-acetyltransferase and AANAT, and typically classified as EC 2.3.1.87. AANAT catalyzes the conversion of acetyl-CoA and serotonin to CoA and N-Acetyl-serotonin (FIG. 1). Exemplary nucleic acids encoding AANAT enzymes for use in aspects and embodiments of the present invention include, but are not limited to, those encoding the AANATs shown in Table 1, as well as functionally active variants, homologs or fragments thereof. Suitable assays for testing N-acetylserotonin production by an AANAT in a recombinant microbial host cell are described in, e.g., Thomas et al., Analytical Biochemistry 1990; 184:228-34.

### Acetylserotonin O-Methyltransferase

In one aspect, the recombinant cell further comprises an exogenous nucleic acid encoding an acetylserotonin O-methyltransferase or ASMT, typically classified as EC 2.1.1.4. ASMT catalyzes the last reaction in the production of melatonin from L-tryptophan, the conversion of N-acetyl-serotonin and S-adenosyl-L-methionine (SAM) to Melatonin and S-adenosyl-L-homocysteine (SAH) (FIG. 1). As described herein, SAH can then be recycled back to SAM via the S-adenosyl-L-methionine cycle in microbial cells where the S-adenosyl-L-methionine cycle is native (or exogenously added) and constitutively expressed, such as, e.g., in E. coli. Exemplary nucleic acids encoding ASMT enzymes for use in aspects and embodiments of the present invention include, but are not limited to, those encoding ASMTs shown in Table 1, as well as functionally active variants, homologs or fragments thereof. Suitable assays for testing melatonin production by an ASMT in a recombinant microbial host cell have been described in, e.g., Kang et al. (2011), which is hereby incorporated by reference in its entirety.

TABLE 1
Exemplary enzymes and amino acid sequences

| Name (EC #) | Species | SEQ ID # |
|---|---|---|
| L-tryptophan hydroxylase (EC | Oryctolagus cuniculus TPH1 | 1 |
| 1.14.16.4) (TPH) | Homo sapiens TPH1 | 2 |
| | Homo sapiens TPH2 | 3 |
| | Bos taurus | 4 |
| | Sus scrofa | 5 |
| | Gallus gallus | 6 |
| | Mus musculus | 7 |
| | Equus caballus | 8 |
| | Homo sapiens TPH2, truncated ((45-471) + 20) | 175 |
| | Homo sapiens TPH2, truncated (45-471) | 185 |
| | Homo sapiens TPH2, truncated (146-460) | 176 |
| | Schistosoma mansoni | 177 |
| GTP cyclohydrolase I (EC | Homo sapiens | 10 |
| 3.5.4.16) (GCH1) | Mus musculus | 11 |
| | E. coli | 12 |
| | S. cerevisiae | 13 |
| | Bacillus subtilis | 14 |
| | Streptomyces avermitilis | 15 |
| | Salmonella typhii | 16 |
| 6-pyruvoyl-tetrahydropterin | Homo sapiens | 17 |
| synthase (EC 4.2.3.12) (PTPS) | Rattus norwegicus | 18 |
| | Bacteroides thetaiotaomicron | 19 |
| | Thermosynechococcus elongates | 20 |
| | Streptococcus thermophilus | 21 |
| | Acaryochloris marina | 22 |
| sepiapterin reductase (EC | Homo sapiens | 23 |
| 1.1.1.153) (SPR) | Rattus norwegicus | 24 |
| | Mus musculus | 25 |
| | Bos taurus | 26 |
| | Danio rerio | 27 |
| | Xenopus laevis | 28 |
| pterin-4-alpha-carbinolamine | Pseudomonas aeruginosa | 29 |
| dehydratase (EC 4.2.1.96) | Bacillus cereus var. anthracis | 30 |
| (PCBD1) | Corynebacterium genitalium | 31 |
| | Lactobacillus ruminis ATCC 25644 | 32 |
| | Rhodobacteraceae bacterium HTCC2083 | 33 |
| | Homo sapiens | 34 |
| dihydropteridine reductase (EC | Homo sapiens | 35 |
| 1.5.1.34) (DHPR) | Rattus norwegicus | 36 |
| | Sus scrofa | 37 |
| | Bos taurus | 38 |
| | E. coli | 39 |
| | Dictyostelium discoideum | 40 |
| 5-Hydroxy-L-tryptophan | Acidobacterium capsulatum | 41 |
| decarboxylase (EC 4.1.1.28) | Rattus norwegicus | 42 |
| (DDC) | Sus scrofa | 43 |
| | Homo sapiens | 44 |
| | Capsicum annuum | 45 |
| | Drosophila caribiana | 46 |
| | Maricaulis maris (strain MCS10) | 47 |
| | Oryza sativa subsp. Japonica | 48 |
| | Pseudomonas putida S16 | 49 |
| | Catharanthus roseus | 50 |
| serotonin acetyltransferase (EC | Chlamydomonas reinhardtii | 51 |
| 2.3.1.87 or 2.3.1.5) (AANAT) | Bos Taurus | 52 |
| | Bos Taurus A55P | 178 |
| | Gallus gallus | 53 |
| | Homo sapiens | 54 |
| | Mus musculus | 55 |
| | Oryctolagus cuniculus | 56 |
| | Ovis aries | 57 |
| acetylserotonin O- | Oryza sativa | 58 |
| methyltransferase (EC 2.1.1.4) | Homo sapiens | 59 |
| (ASMT) | Bos Taurus | 60 |
| | Rattus norvegicus | 61 |
| | Gallus gallus | 62 |
| | Macaca mulatta | 63 |
| S-adenosylmethionine synthetase | Escherichia coli | 64 |
| (EC 2.5.1.6) | Saccharomyces cerevisiae (SAM1) | 65 |
| | Saccharomyces cerevisiae (SAM2) | 66 |
| | Bos taurus | 67 |
| | Homo Sapiens | 68 |
| | Bacillus substilis | 69 |
| Ethionine resistance protein | Saccharomyces cerevisiae | 70 |
| | Pichia stipitis | 71 |
| S-adenosylhomocysteine | Saccharomyces cerevisiae | 72 |
| hydrolase (EC 3.3.1.1) | Bos taurus | 73 |
| | Homo Sapiens | 74 |
| Methionine synthase (EC 2.1.1.—) | Escherichia coli | 75 |
| | Saccharomyces cerevisiae | 76 |
| | Bos taurus | 77 |
| | Homo Sapiens | 78 |
| AcCoA synthetase (EC 6.2.1.1) | Escherichia coli | 79 |
| | Salmonella enterica | 80 |
| | Bacillus substilis | 81 |
| Acetylaldehyde dehydrogenase | Klebsiella pneumonia | 82 |
| (EC 1.2.1.3) | Bacillus sp. | 83 |
| | Escherichia coli | 84 |
| Feedback-resistant DAHP | E. coli (AroG-fbr) | 85 |
| synthase (EC 2.5.1.54) | | |
| Transketolase (EC 2.2.1.1) | Escherichia coli | 86 |
| | Saccharomyces cerevisiae | 87 |
| | Kluyveromyces lactis | 88 |
| PEP synthase (EC 2.7.9.2) | Escherichia coli | 89 |
| | Enterobacter agglomerans | 90 |
| Hexokinase (EC 2.7.1.1) | Saccharomyces cerevisiae | 91 |
| | Kluyveromyces lactis | 92 |
| | Aspergillus oryzae | 93 |
| Glucose facilitated diffusion | Zymomonas mobilis subsp. mobilis (strain ATCC | 94 |
| protein (TC 2.A.1.1) | 31821/ZM4/CP4) | |

Variants or homologs of any one or more of the enzymes and other proteins listed in Table 1, having the referenced activity and a sequence identity of at least 30%, such as at least 50%, such as at least 60%, such as at least 70%, such as at least 80%, such as at least 90%, such as at least 95%, such as at least 99%, over at least the catalytically active portion, optionally over the full length, of the reference amino acid sequence, are also contemplated. The variant or homolog may comprise, for example, 2, 3, 4, 5 or more, such as 10 or more, amino acid substitutions, insertions or deletions as compared to the reference amino acid sequence. In particular conservative substitutions are considered. These are typically within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In: The Proteins, Academic Press, New York. The most commonly occurring exchanges are Ala to Ser, Val to Ile, Asp to Glu, Thr to Ser, Ala to Gly, Ala to Thr, Ser to Asn, Ala to Val, Ser to Gly, Tyr to Phe, Ala to Pro, Lys to Arg, Asp to Asn, Leu to Ile, Leu to Val, Ala to Glu, and Asp to Gly. Homologs, such as orthologs or paralogs, having the desired activity can be identified in the same or a related animal or microbial species using the reference sequences provided and appropriate activity testing.

An exogenously added nucleic acid sequence encoding an enzyme or other protein activity listed in Table 1 may encode an amino acid sequence that is homologous (i.e., native) or heterologous to the recombinant host cell in question.

In one embodiment, the recombinant microbial cell comprises exogenous nucleic acid sequences encoding at least one, two, three, four, five, six, seven, eight or more or the amino acid sequences listed in Table 1. In one specific embodiment, the recombinant microbial cell comprises exogenous nucleic acids encoding a TPH, a PTS, an SPR, a DDC, an AANAT, an ASMT, and, optionally, a DHPR, a PCBD1 and/or a GCH1, selected from those in Table 1. In one embodiment, the recombinant microbial cell further comprises an S-adenosyl-methionine synthetase, an ethionine resistance protein, an S-adenosylhomocysteine hydrolase, a methionine synthase, an AcCoA synthetase, an acetylaldehyde dehydrogenase, a 3-Deoxy-d-heptulosonate-7-phosphate (DAHP) synthase, a transketolase, a PEP synthase, a hexokinase and glucose facilitated diffusion protein selected from those in Table 1, or a combination of two or more of different protein or enzyme activities.

In a specific embodiment, the recombinant cell is a yeast cell such as, e.g., a S. cerevisiae cell, comprising exogenous nucleic acids encoding a TPH comprising SEQ ID NO:176 or 177 (TPH), or a functional truncated or mutated version of any thereof, as well as exogenous nucleic acids encoding a PTS, an SPR, a DDC, an AANAT and an ASMT, each optionally separately selected from the relevant group in Table 1. The cell may optionally also comprise exogenous nucleic acids encoding a DHPR, a PCBD1, and/or a GCH1. In a preferred embodiment the recombinant yeast cell further comprises a deletion of the aro9 gene or ortholog thereof. In another specific embodiment, the recombinant cell comprises exogenous nucleic acids encoding SEQ ID NOS:175 (TPH), 18 (PTS), 24 (SPR), 39 (DHPR), 34 (PCBD1), 44 (DDC), 52 (AANAT), 62 (ASMT), and, optionally, 12 (GCH1). In another specific embodiment, the recombinant cell is a yeast cell such as, e.g., a S. cerevisiae cell, comprising comprises exogenous nucleic acids encoding SEQ ID NOS:176 (TPH), 18 (PTS), 24 (SPR), optionally one of 35 and 39 (DHPR), optionally one of 32 and 34 (PCBD1), 44 (DDC), 178 (AANAT), one of 62 or 59 (ASMT), and, optionally, 12 (GCH1). In another specific embodiment, the recombinant cell is a yeast cell such as, e.g., a S. cerevisiae cell, comprising exogenous nucleic acids encoding SEQ ID NOS:177 (TPH), 18 (PTS), 24 (SPR), optionally one of 35 and 39 (DHPR), optionally one of 32 and 34 (PCBD1), 44 (DDC), 178 (AANAT), one of 59 or 62 (ASMT), and, optionally, 12 (GCH1). In a preferred embodiment, one or more, preferably all of the exogenous nucleic acids is each under the control of a strong promoter, e.g., each separately selected from PGK1, TEF1, HXT7 and TDH3.

### Vectors

The invention also provides one or more vectors comprising nucleic acid sequences according to the above embodiments, e.g., encoding one, two, three, four, five, six or more enzymes selected from TPH, DDC, AANAT, ASMT, PTPS, SPR, GCH1, PCBD1 and DHPR. Optionally, the vector or vectors further comprise nucleic acid sequence encoding one or more enzymes or other proteins having activities selected from

S-adenosylmethionine synthetase (EC 2.5.1.6)

ethionine resistance gene product,

S-adenosylhomocysteine hydrolase (EC 3.3.1.1),

methionine synthase (EC 2.1.1.-),

AcCoA synthetase (EC 6.2.1.1),

acetylaldehyde dehydrogenase (EC 1.2.1.3)

3-deoxy-d-heptulosonate-7-phosphate (DAHP) synthase (EC 2.5.1.54)

transketolase (EC 2.2.1.1)

PEP synthase (EC 2.7.9.2),

hexokinase (EC 2.7.1.1),

glucose facilitated diffusion protein (TC 2.A.1.1), or

a combination of any two or more thereof, optionally wherein the enzymes or other proteins comprise amino acid sequences selected from those in Table 1.

The specific design of the vector depends on whether the intended microbial host cell is to be provided with one or both THB pathways, as well as on whether host cell endogenously produces sufficient amounts of one or more of the enzymes of the THB pathways. For example, for an S. cerevisiae host cell, it may not be necessary to include a nucleic acid sequence encoding a GCH1, since the enzyme is native to S. cerevisiae at a sufficient activity (see Example 1). Additionally, for transformation of a particular host cell, two or more vectors with different combinations of the enzymes used in the present invention can be applied. The vector can be a plasmid, phage vector, viral vector, episome, an artificial chromosome or other polynucleotide construct, and may, for example, include one or more selectable marker genes and appropriate regulatory control sequences.

Generally, regulatory control sequences are operably linked to the encoding nucleic acid sequences, and include constitutive, regulatory and inducible promoters, transcription enhancers, transcription terminators, and the like which are well known in the art. The encoding nucleic acid sequences can be operationally linked to one common expression control sequence or linked to different expression control sequences, such as one inducible promoter and one constitutive promoter.

The procedures used to ligate the various regulatory control and marker elements with the encoding nucleic acid sequences to construct the vectors of the present invention are well known to one skilled in the art (see, e.g., Sambrook et al., 2012, supra). In addition, methods have recently been developed for assembling of multiple overlapping DNA molecules (Gibson et al., 2008) (Gibson et al., 2009) (Li & Elledge, 2007), allowing, e.g., for the assembly multiple overlapping DNA fragments by the concerted action of an exonuclease, a DNA polymerase and a DNA ligase.

The promoter sequence is typically one that is recognized by the intended host cell. For an E. coli host cell, suitable promoters include, but are not limited to, the lac promoter, the T7 promoter, pBAD, the tet promoter, the Lac promoter, the Trc promoter, the Trp promoter, the recA promoter, the λ (lambda) promoter, and the PL promoter. For Streptomyces host cells, suitable promoters include that of Streptomyces coelicolor agarase (dagA). For a Bacillus host cell, suitable promoters include the sacB, amyL, amyM, amyQ, penP, xylA and xylB. Other promoters for bacterial cells include prokaryotic beta-lactamase (Villa-Kamaroff et al., 1978, Proceedings of the National Academy of Sciences USA 75: 3727-3731), and the tac promoter (DeBoer et al., 1983, Proceedings of the National Academy of Sciences USA 80: 21-25). For an S. cerevisiae host cell, useful promoters include the TEF1, HXT7, TDH3, ENO-1, GAL1, ADH1, ADH2, GAP, TPI, CUP1, PHO5 and PGK, such as PGK1 promoters. Other useful promoters for yeast host cells are described by Romanos et al., 1992, Yeast 8: 423-488. Still other useful promoters for various host cells are described in “Useful proteins from recombinant bacteria” in Scientific American, 1980, 242: 74-94; and in Sambrook et al., 2012, supra.

A transcription terminator sequence is a sequence recognized by a host cell to terminate transcription, and is typically operably linked to the 3′ terminus of an encoding nucleic acid sequence. Suitable terminator sequences for E. coli host cells include the T7 terminator region. Suitable terminator sequences for yeast host cells such as S. cerevisiae include CYC1, PGK, GAL, ADH, AOX1 and GAPDH. Other useful terminators for yeast host cells are described by Romanos et al., 1992, supra.

A leader sequence is a non-translated region of an mRNA which is important for translation by the host cell. The leader sequence is typically operably linked to the 5′ terminus of a coding nucleic acid sequence. Suitable leaders for yeast host cells include S. cerevisiae ENO-1, PGK, alpha-factor, ADH2/GAP, TEF, and Kozak sequence.

A polyadenylation sequence is a sequence operably linked to the 3′ terminus of a coding nucleic acid sequence which, when transcribed, is recognized by the host cell as a signal to add polyadenosine residues to transcribed mRNA. Useful polyadenylation sequences for yeast host cells are described by Guo and Sherman, 1995, Mol Cell Biol 15: 5983-5990.

A signal peptide sequence encodes an amino acid sequence linked to the amino terminus of an encoded amino acid sequence, and directs the encoded amino acid sequence into the cell's secretory pathway. In some cases, the 5′ end of the coding nucleic acid sequence may inherently contain a signal peptide coding region naturally linked in translation reading frame, while a foreign signal peptide coding region may be required in other cases. Useful signal peptides for yeast host cells can be obtained from the genes for S. cerevisiae alpha-factor and invertase. Other useful signal peptide coding regions are described by Romanos et al., 1992, supra. An exemplary signal peptide for an E. coli host cell can be obtained from alkaline phosphatase. For a Bacillus host cell, suitable signal peptide sequences can be obtained from alpha-amylase and subtilisin. Further signal peptides are described by Simonen and Palva, 1993, Microbiological Reviews 57: 109-137.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound.

Regulatory systems in prokaryotic systems include the lac, tec, and tip operator systems. For example, one or more promoter sequences can be under the control of an IPTG inducer, initiating expression of the gene once IPTG is added. In yeast, the ADH2 system or GAL1 system may be used. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the respective encoding nucleic acid sequence would be operably linked with the regulatory sequence.

The choice of the vector will typically depend on the compatibility of the vector with the host cell into which the vector is to be introduced. The vectors may be linear or closed circular plasmids. The vector may also be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. The selectable marker genes can, for example, provide resistance to antibiotics or toxins, complement auxotrophic deficiencies, or supply critical nutrients not in the culture media, and/or provide for control of chromosomal integration. Examples of bacterial selectable markers are the dal genes from Bacillus subtilis or Bacillus licheniformis, or markers which confer antibiotic resistance such as ampicillin, kanamycin, chloramphenicol, or tetracycline resistance. Suitable markers for yeast host cells are ADE2, HIS3, LEU2, LYS2, MET3, TRP1, and URA3.

The vectors of the present invention may also contain one or more elements that permit integration of the vector into the host cell genome or autonomous replication of the vector in the cell independent of the genome. For integration into the host cell genome, the vector may rely on an encoding nucleic acid sequence or other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination. The integrational elements may be any sequence that is homologous with the target sequence in the genome of the host cell. The integrational elements may, for example, non-encoding or encoding nucleotide sequences. The vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo. Examples of bacterial origins of replication are the origins of replication of plasmids pBR322, pUC19, pACYC177, and pACYC184 permitting replication in E. coli, and pUB1 10, pE194, pTA1060, and pAMβi permitting replication in Bacillus. Examples of origins of replication for use in a yeast host cell are the 2 micron origin of replication, ARS1, ARS4, the combination of ARS1 and CEN3, and the combination of ARS4 and CEN6.

More than one copy of the nucleic acid sequence encoding the L-tryptophane hydroxylase, DDC, TDC, TSH, AANAT, ASMT, SPR, DHPR, GCH1, PTPS, PCBD1, S-adenosylmethionine synthetase, ethionine resistance gene product, S-adenosylhomocysteine hydrolase, methionine synthase, AcCoA synthetase, acetylaldehyde dehydrogenase, 3-deoxy-d-heptulosonate-7-phosphate (DAHP) synthase, transketolase, PEP synthase, hexokinase or glucose facilitated diffusion protein may be inserted into the host cell to increase production of the gene product. An increase in the copy number of the encoding nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the sequence, can be selected for by cultivating the cells in the presence of the appropriate selectable agent.

### Recombinant Host Cells

The present invention also provides a recombinant host cell, into which one or more vectors according to any preceding embodiment is introduced, typically via transformation, using standard methods known in the art (see, e.g., Sambrook et al., 2012, supra. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizen, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thome, 1987, Journal of Bacteriology 169: 5771-5278).

As described above, the vector, once introduced, may be maintained as a chromosomal integrant or as a self-replicating extra-chromosomal vector.

Preferably, for transformation of an E. coli or other bacterial host cell, the vectors are designed as follows: A lac promoter is used to control the expressions of a gene or an artificial operon containing up to three genes connected with a linker sequence, in order to express the genes at a suitable level so that the introduction of heterologous genes/pathways do not overdraw substrates or energy in the host cell. In one particular embodiment, the recombinant microbial cell, preferably a bacterial cell, is transformed according to a strategy outlined in the Examples.

Preferably, for transformation of a yeast host cell such as S. cerevisiae, the heterologous genes are integrated onto chromosome using a homologous recombination based method (Mikkelsen et al., 2012). As compared with gene expression based on plasmids, the chromosomal integrated genes can be expressed with higher fidelity and resulted in better protein translation, in particular for multiple gene co-expression systems. In one particular embodiment, the recombinant microbial cell, preferably a yeast cell, is transformed according to a strategy outlined in the Examples.

The transformation can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product, including those referred to above and relating to measurement of 5HTP production. Expression levels can further be optimized to obtain sufficient expression using methods well known in the art and as disclosed herein.

Tryptophan production takes place in all known microorganisms by a single metabolic pathway (Somerville, R. L., Herrmann, R. M., 1983, Amino acids, Biosynthesis and Genetic Regulation, Addison-Wesley Publishing Company, U.S.A.: 301-322 and 351-378; Aida et al., 1986, Bio-technology of amino acid production, progress in industrial microbiology, Vol. 24, Elsevier Science Publishers, Amsterdam: 188-206). The recombinant microbial cell of the invention can thus be prepared from any microbial host cell, using recombinant techniques well known in the art (see, e.g., Sambrook et al., 2012, supra, and Ausubel et al. (1991), supra. Preferably, the host cell is tryptophan autotrophic (i.e., capable of endogenous biosynthesis of L-tryptophan), grows on synthetic medium with suitable carbon sources, and expresses a suitable RNA polymerase (such as, e.g., T7 polymerase).

The microbial host cell for use in the present invention is typically unicellular and can be, for example, a bacterial cell, a yeast host cell, a filamentous fungal cell, or an algeal cell. Examples of suitable host cell genera include, but are not limited to, Acinetobacter, Agrobacterium, Alcaligenes, Anabaena, Aspergillus, Bacillus, Bifidobacterium, Brevibacterium, Candida, Chlorobium, Chromatium, Corynebacteria, Cytophaga, Deinococcus, Enterococcus, Erwinia, Erythrobacter, Escherichia, Flavobacterium, Hansenula, Klebsiella, Lactobacillus, Methanobacterium, Methylobacter, Methylococcus, Methylocystis, Methylomicrobium, Methylomonas, Methylosinus, Mycobacterium, Myxococcus, Pantoea, Phaffia, Pichia, Pseudomonas, Rhodobacter, Rhodococcus, Saccharomyces, Salmonella, Sphingomonas, Streptococcus, Streptomyces, Synechococcus, Synechocystis, Thiobacillus, Trichoderma, Yarrowia and Zymomonas.

In one embodiment, the host cell is bacterial cell, e.g., an Escherichia cell such as an Escherichia coli cell; a Bacillus cell such as a Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, or a Bacillus thuringiensis cell; or a Streptomyces cell such as a Streptomyces lividans or Streptomyces murinus cell. In a particular embodiment, the host cell is an E. coli cell. In another particular embodiment, the host cell is of an E. coli strain selected from the group consisting of K12.DH1 (Proc. Natl. Acad. Sci. USA, volume 60, 160 (1968)), JM101, 3M103 (Nucleic Acids Research (1981), 9, 309), 3A221 (3. Mol. Biol. (1978), 120, 517), HB101 (3. Mol. Biol. (1969), 41, 459) and C600 (Genetics, (1954), 39, 440).

In one embodiment, the host cell is a fungal cell, such as, e.g., a yeast cell. Exemplary yeast cells include Candida, Hansenula, Kluyveromyces, Pichia, Saccharomyces, Schizosaccharomyces and Yarrowia cells. In a particular embodiment, the host cell is an S. cerevisiae cell. In another particular embodiment, the host cell is of an S. cerevisiae strain selected from the group consisting of S. cerevisiae KA31, AH22, AH22R-, NA87-11A, DKD-5D and 20B-12, S. pombe NCYC1913 and NCYC2036 and Pichia pastoris KM71.

### Production of Melatonin or Related Compounds

The invention also provides a method of producing melatonin, serotonin and/or N-acetyl-serotonin, comprising culturing the recombinant microbial cell of any preceding aspect or embodiment in a medium comprising a carbon source. The desired compound can then optionally be isolated or retrieved from the medium, and optionally further purified. Importantly, using a recombinant microbial cell according to the invention, the method can be carried out without adding L-tryptophan, THB, or both, to the medium.

Also provided is a method of preparing a composition comprising one or more compounds selected from 5HTP, serotonin and/or N-acetyl-serotonin, comprising culturing the recombinant microbial cell of any preceding aspect or embodiment, isolating and purifying the compound(s), and adding any excipients to obtain the composition.

Suitable carbon sources include carbohydrates such as monosaccharides, oligosaccharides and polysaccharides. As used herein, “monosaccharide” denotes a single unit of the general chemical formula Cx(H2O)y, without glycosidic connection to other such units, and includes glucose, fructose, xylose, arabinose, galactose and mannose. “Oligosaccharides” are compounds in which monosaccharide units are joined by glycosidic linkages, and include sucrose and lactose. According to the number of units, oligosaccharides are called disaccharides, trisaccharides, tetrasaccharides, pentasaccharides etc. The borderline with polysaccharides cannot be drawn strictly; however the term “oligosaccharide” is commonly used to refer to a defined structure as opposed to a polymer of unspecified length or a homologous mixture. “Polysaccharides” is the name given to a macromolecule consisting of a large number of monosaccharide residues joined to each other by glycosidic linkages, and includes starch, lignocellulose, cellulose, hemicellulose, glycogen, xylan, glucuronoxylan, arabinoxylan, arabinogalactan, glucomannan, xyloglucan, and galactomannan. Other suitable carbon sources include acetate, glycerol, pyruvate and gluconate. In one embodiment, the carbon source is selected from the group consisting of glucose, fructose, sucrose, xylose, mannose, galactose, rhamnose, arabinose, fatty acids, glycerine, glycerol, acetate, pyruvate, gluconate, starch, glycogen, amylopectin, amylose, cellulose, cellulose acetate, cellulose nitrate, hemicellulose, xylan, glucuronoxylan, arabinoxylan, glucomannan, xyloglucan, lignin, and lignocellulose. In one embodiment, the carbon source comprises one or more of lignocellulose and glycerol. In one embodiment, the carbon source is a simple carbon source such as glucose, xylose, fructose, arabinose, galactose, mannose, glycerol, acetate, or a mixture of any thereof.

The culture conditions are adapted to the recombinant microbial host cell, and can be optimized to maximize production or melatonin or a related compound by varying culture conditions and media components as is well-known in the art.

For a recombinant Escherichia coli cell, exemplary media include LB medium and M9 medium (Miller, Journal of Experiments in Molecular Genetics, 431-433, Cold Spring Harbor Laboratory, New York, 1972), optionally supplemented with one or more amino acids. When an inducible promoter is used, the inductor can also be added to the medium. Examples include the lac promoter, which can be activated by adding isopropyl-beta-thiogalacto-pyranoside (IPTG) and the GAL/BAD promoter, in which case galactose/arabinose can be added. The culturing can be carried out a temperature of about 10 to 40° C. for about 3 to 72 hours, if desired, with aeration or stirring.

For a recombinant Bacillus cell, culturing can be carried out in a known medium at about 30 to 40° C. for about 6 to 40 hours, if desired with aeration and stirring. With regard to the medium, known ones may be used. For example, pre-culture can be carried out in an LB medium and then the main culture using an NU medium.

For a recombinant yeast cell, Burkholder minimum medium (Bostian, K. L., et al. Proc. Natl. Acad. Sci. USA, volume 77, 4505 (1980)), SD medium containing 0.5% of Casamino acid (Bitter, G. A., et al., Proc. Natl. Acad. Sci. USA, volume 81, 5330 (1984), and Delft medium (Verduyn et al., Yeast 1992, 8, 501-517) can be used. The pH is preferably adjusted to about 5-8. For example, a synthetic medium may contain, per litre: (NH4)2SO4, 5 g; KH2PO4, 3 g; MgSO4.7H2O, 0.5 g; EDTA, 15 mg; ZnSO4.7H2O, 4.5 mg; CoCl2.6H2O, 0.3 mg; MnCl2.4H20, 1 mg; CuSO4 5H2O, 0.3 mg; CaCl2.2H2O, 4.5 mg; FeSO4.7H2O, 3 mg; NaMoO4.2H2O, 0.4 mg; H3B03, 1 mg-KI, 0.1 mg; and 0.025 ml silicone antifoam (BDH). Filter-sterilized vitamins can be added after heat sterilization (120° C.), to final concentrations per litre of: biotin, 0.05 mg; calcium pantothenate, 1 mg; nicotinic acid, 1 mg; inositol, 25 mg; thiamine HCl, 1 mg; pyridoxine HCl, 1 mg; and para-aminobenzoic acid, 0.2 mg. The medium can then be adjusted to pH6 with KOH. Culturing is preferably carried out at about 20 to about 40° C., for about 24 to 84 hours, if desired with aeration or stirring.

In one embodiment, no L-tryptophan is added to the medium. In another embodiment, no L-tryptophan or THB is added to the medium, so that the production of melatonin or its precursors or related compounds rely on endogenously biosynthesized substrates. In one embodiment, the medium is supplemented with methionine, e.g., at about 0.01 to about 10 g/L, such as about 0.02 to about 5 g/L, such as about 0.03 to about 3 g/L, such as about 0.1 to 1 g/L, or at least about 0.05 g/L, or at least about 0.1 g/L. In one embodiment, the medium is supplemented with SAM, e.g., at about 0.01 to about 1.5 g/L, such as about 0.1 to about 1 g/L, such as at least about 0.05 g/L or at least about 0.1 g/L. In one embodiment, the culture medium is supplemented with both methionine and SAM, as described above.

Using the method for producing melatonin, serotonin or N-acetyl-serotonin according to the invention, a melatonin yield of at least about 0.5%, such as at least about 1%, such as at least 5%, such as at least 10%, such as at least 20%, at least 30%, at least 40%, at least 50%, at least 60%, at least 70%, at least 80% or at least 90% of the theoretically possible yield can be obtained from a suitable carbon source, such as glucose.

Isolation of melatonin, N-acetylserotonin or serotonin from the cell culture can be achieved, e.g., by separating the compound from the cells using a membrane, using, for example, centrifugation or filtration methods. The product-containing supernatant is then collected. Further purification of the desired compound can then be carried out using known methods, such as, e.g., salting out and solvent precipitation; molecular-weight-based separation methods such as dialysis, ultrafiltration, and gel filtration; charge-based separation methods such as ion-exchange chromatography; and methods based on differences in hydrophobicity, such as reversed-phase HPLC; and the like. In one embodiment, ion-exchange chromatography is used for purification of serotonin. An exemplary method for serotonin purification using cation-exchange chromatography is described in Chilcote (1974) (Clin Chem 20(4):421-423). In one embodiment, reverse-phase chromatography is used for separation and/or purification of serotonin, N-acetylserotonin, or melatonin. An exemplary method for purification of these indolamines using reversed-phase chromatography is described in Harumi et al., (1996) (3 Chromatogr B 675:152-156).

Once a sufficiently pure preparation has been achieved, suitable excipients, stabilizers can optionally be added and the resulting preparation incorporated in a composition for use in preparing a product such as, e.g., a dietary supplement, a pharmaceutical, a cosmeceutical, or a nutraceutical. For a dietary supplement comprising melatonin, each serving can contain, e.g., from about 0.01 mg to about 100 mg melatonin, such as from about 0.1 mg to about 10 mg, or about 1-5 mg, such as 2-3 mg. Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives may also be added to the nutritional supplement to extend product shelf life. Preferably, preservatives such as potassium sorbate, sodium sorbate, potassium benzoate, sodium benzoate or calcium disodium EDTA are used.

TABLE 2
Primers used for
cloning in the Examples

| Primer | Sequence (5′-3′) |
|---|---|
| GgTPH-fw (ID1599) | ATCTGTCAUAAAACAATGCACAT |
| | CGAGTCACGGAAATCC |
| | (SEQ ID NO: 95) |
| GgTPH-rv (ID1600) | CACGCGAUTTAAACCTCCAGCTG |
| | CTTGCC (SEQ ID NO: 96) |
| MmTPH2-fw (ID1597) | ATCTGTCAUAAAACAATGGATGA |
| | CAAAGGCAACAAAGGC |
| | (SEQ ID NO: 162) |
| MmTPH2-rv (ID1598) | CACGCGAUTTATACGCAGATCCT |
| | GAACCAC |
| | (SEQ ID NO: 163) |
| SmTPH fw (ID8502) | ATCTGTCAUAAAACAATGATTAG |
| | CACCGAAAGCG |
| | (SEQ ID NO: 164) |
| SmTPH rv (ID8503) | CACGCGAUTTAGCTGCTGCGATT |
| | TTCG (SEQ ID NO: 165) |
| HsTPH2-(146-460) | ATCTGTCAUAAAACAATGGAACT |
| fw (ID8504) | GGAAGATGTTCCG |
| | (SEQ ID NO: 166) |
| HsTPH2-(146-460) | CACGCGAUTTAGGTATCTTTCAG |
| rv (ID8505) | GATCTCGATG |
| | (SEQ ID NO: 167) |
| HsPCBD1-fw (ID2098) | AGTGCAGGUAAAACAATGGCAGG |
| | TAAAGCACATC |
| | (SEQ ID NO: 97) |
| HsPCBD1-rv (ID2099) | CGTGCGAUTTAGCAGCCGGATCA |
| | AAC (SEQ ID NO: 98) |
| LrPCBD1-fw (ID2150) | AGTGCAGGUAAAACAATGGTCAA |
| | GTTGTTCCCATC |
| | (SEQ ID NO: 168) |
| LrPCBD1-rv (ID2151) | CGTGCGAUTCAAATTCTGGCATC |
| | TTGAATTTC |
| | (SEQ ID NO: 169) |
| DHPR-fw (ID395) | AGTGCAGGUAAAACAATGGATAT |
| | CATTTCTGTCG |
| | (SEQ ID NO: 99) |
| DHPR-rv (ID390) | CGTGCGAUTTACACTTCGGTTAA |
| | GGT (SEQ ID NO: 100) |
| HsDHPR-fw (ID2152) | ATCTGTCAUAAAACAATGGCTGC |
| | TGCTGC (SEQ ID NO: 170) |
| HsDHPR-rv (ID2153) | CACGCGAUTTAGAAGTAAGCTGG |
| | AGTC (SEQ ID NO: 171) |
| RnSPR-fw (ID394) | ATCTGTCAUAAAACAATGGAAGG |
| | AGGCAGGCTAG |
| | (SEQ ID NO: 103) |
| RnSPR-rv (ID389) | CACGCGAUTTAAATGTCATAGAA |
| | GTCCACGTG |
| | (SEQ ID NO: 101) |
| RnPTS-fw (ID393) | AGTGCAGGUAAAACAATGAACGC |
| | GGCGGTTGG |
| | (SEQ ID NO: 102) |
| RnPTS_rv (ID350) | CGTGCGAUTTATTCTCCTTTGTA |
| | GACCACAAT |
| | (SEQ ID NO: 172) |
| HsDDC-rv (ID1759) | CGTGCGAUTTATTCACGTTCGGC |
| | ACGCAGCAC |
| | (SEQ ID NO: 104) |
| HsDDC-fw (ID1760) | AGTGCAGGUAAAACAATGAATGC |
| | AAGCGAATTTCGTCG |
| | (SEQ ID NO: 105) |
| BtAANAT-fw (ID1761) | ATCTGTCAUAAAACAATGAGCAC |
| | CCCGAGCATTCATTG |
| | (SEQ ID NO: 106) |
| BtAANAT-rv (ID1762) | CACGCGAUTTAACGATCGCTATT |
| | ACGACGCAGTG |
| | (SEQ ID NO: 107) |
| GgASMT-fw (ID1764) | AGTGCAGGUAAAACAATGGATAG |
| | CACCGAAGATCTGG |
| | (SEQ ID NO: 108) |
| GgASMT-rv (ID1763) | CGTGCGAUTTATTTACGACCCAG |
| | AACTGCATC |
| | (SEQ ID NO: 109) |
| HsASMT-fw (ID2254) | AGTGCAGGUAAAACAATGGGTAG |
| | CAGCGAAGATC |
| | (SEQ ID NO: 173) |
| HsASMT-rv (ID2255) | CGTGCGAUTTATTTACGTGCCAG |
| | GATTGCATC |
| | (SEQ ID NO: 174) |
| H1-P1-tnaA | ATGGAAAACTTTAAACATCTCCC |
| | TGAACCGTTCCGCATTCGTGTAG |
| | GCTGGAGCTGCTTC |
| | (SEQ ID NO: 110) |
| H2-P2-tnaA | TCGGTTCGTACGTAAAGGTTAAT |
| | CCTTTAATATTCGCCGCATATGA |
| | ATATCCTCCTTAG |
| | (SEQ ID NO: 111) |
| K1 | CAGTCATAGCCGAATAGCCT |
| | (SEQ ID NO: 112) |
| CFB-FWD-tnaA | GGCGAATTAATCGGTATAGCAGA |
| | TG (SEQ ID NO: 113) |
| ACS1-FWD | AGTGCAGGUAAAACAATGTCGCC |
| | CTCTGCCGTACA |
| | (SEQ ID NO: 114) |
| ACS1-REV | CGTGCGAUTTACAACTTGACCGA |
| | ATCAA (SEQ ID NO: 115) |
| ALD6-FWD | AGTGCAGGUAAAACAATGACTAA |
| | GCTACACTTTG |
| | (SEQ ID NO: 116) |
| ALD6-REV | CGTGCGAUTTCAGTGTATGCATG |
| | G (SEQ ID NO: 117) |
| PTEF1-FW (ID5) | ACCTGCACUTTGTAATTAAAACT |
| | TAG (SEQ ID NO: 118) |
| PTEF1-RV (ID6) | CACGCGAUGCACACACCATAGCT |
| | TC (SEQ ID NO: 119) |
| PPGK1_FW (ID7) | CGTGCGAUGGAAGTACCTTCAAA |
| | GA (SEQ ID NO: 120) |
| PPGK1_RV (ID8) | ATGACAGAUTTGTTTTATATTTG |
| | TTG (SEQ ID NO: 121) |
| SAM2-FWD | AGTGCAGGUAAAACAATGTCCAA |
| | GAGCAAAAC |
| | (SEQ ID NO: 122) |
| SAM2-REV | CACGCGAUTTAAAATTCCAATTT |
| | C (SEQ ID NO: 123) |
| SAH1-FWD | ATCTGTCAUAAAACAATGTCTGC |
| | TCCAGCTC |
| | (SEQ ID NO: 124) |
| SAH1-REV | CACGCGAUTCAATATCTGTAGTG |
| | (SEQ ID NO: 125) |
| Met6-FWD | AGTGCAGGUAAAACAATGGTTCA |
| | ATCTG (SEQ ID NO: 126) |
| Met6-Rev | CGTGCGAUTTAATTCTTGTATTG |
| | TTC (SEQ ID NO: 127) |
| ERC1-fwd | ATCTGTCAUAAAACAATGTCTAA |
| | ACAATTTAGTC |
| | (SEQ ID NO: 128) |
| ERC1-rev | CACGCGAUCTAGTTATACCCAAC |
| | CATAAG (SEQ ID NO: 129) |
| H1-P1-trpR | ATGGCCCAACAATCACCCTATTC |
| | AGCAGCGATGGCAGAACGTGTAG |
| | GCTGGAGCTGCTTC |
| | (SEQ ID NO: 130) |
| H2-P2-trpR | TCAATCGCTTTTCAGCAACACCT |
| | CTTCCAGCCACTGGCCATATGAA |
| | TATCCTCCTTAG |
| | (SEQ ID NO: 131) |
| trpR-cfm | GAGCGCCACGGAATG |
| | (SEQ ID NO: 132) |
| H1-P1-ptsH | ATGTTCCAGCAAGAAGTTACCAT |
| | TACCGCTCCGAACGGTGTAGGCT |
| | GGAGCTGCTTC |
| | (SEQ ID NO: 133) |
| H2-P2-ptsH | TTACTCGAGTTCCGCCATCAGTT |
| | TAACCAGATGTTCAACCCATATG |
| | AATATCCTCCTTAG |
| | (SEQ ID NO: 134) |
| pK18-Fwd | ACTGGCCGTCGTTTTACAACG |
| | (SEQ ID NO: 135) |
| pK18-Rev | CATGGTCATAGCTGTTTCCTGTG |
| | (SEQ ID NO: 136) |
| aroG-Fwd | CAGGAAACAGCTATGACCATGAA |
| | TTATCAGAACGAC |
| | (SEQ ID NO: 137) |
| aroG- Rev | TGTAAAACGACGGCCAGTTTACC |
| | CGCGACGCGCTTTTACTG |
| | (SEQ ID NO: 138) |
| pAroG-Fwd | GACTGGGAAAACCCTG |
| | (SEQ ID NO: 139) |
| pAroG-Rev | TGTAAAACGACGGCCAGTTTAC |
| | (SEQ ID NO: 140) |
| tktA-fwd | GCCGTCGTTTTACAATGTCCTCA |
| | CGTAAAG |
| | (SEQ ID NO: 141) |
| tktA-rev | GAGCCATTGTTGGACATACGACG |
| | TTACAGCAGTTCTTTT |
| | (SEQ ID NO: 142) |
| ppsA-fwd | AAAAGAACTGCTGTAACGTCGTA |
| | TGTCCAACAATGGCTC |
| | (SEQ ID NO: 143) |
| ppsA-rev | GCGATTAAGTTGGGTAACGCCAG |
| | GGTTTTCCCAG |
| | (SEQ ID NO: 144) |

The following enzyme sequences were used in the Examples:

TPH2, truncated 45-471(+20) (Homo sapiens)
(Examples 1 and 7):
(SEQ ID NO: 175)
MDDKGNKGSSKREAATESGKTAVVFSLKNEVGGLVKALRLFQEKRVN
MVHIESRKSRRRSSEVEIFVDCECGKTEFNELIQLLKFQTTIVTLNP
PENIWTEEEELEDVPWFPRKISELDKCSHRVLMYGSELDADHPGFKD
NVYRQRRKYFVDVAMGYKYGQPIPRVEYTEEETKTWGVVFRELSKLY
PTHACREYLKNFPLLTKYCGYREDNVPQLEDVSMFLKERSGFTVRPV
AGYLSPRDFLAGLAYRVFHCTQYIRHGSDPLYTPEPDTCHELLGHVP
LLADPKFAQFSQEIGLASLGASDEDVQKLATCYFFTIEFGLCKQEGQ
LRAYGAGLLSSIGELKHALSDKACVKAFDPKTTCLQECLITTFQEAY
FVSESFEEAKEKMRDFAKSITRPFSVYFNPYTQSIEILKDTRSIENV
VQDLRINRVHSSALTEKEGVRQPEV*
TPH2, truncated 146-460 (Homo sapiens)
(Example 7):
(SEQ ID NO: 176)
MELEDVPWFPRKISELDKCSHRVLMYGSELDADHPGFKDNVYRQRRK
YFVDVAMGYKYGQPIPRVEYTEEETKTWGVVFRELSKLYPTHACREY
LKNFPLLTKYCGYREDNVPQLEDVSMFLKERSGFTVRPVAGYLSPRD
FLAGLAYRVFHCTQYIRHGSDPLYTPEPDTCHELLGHVPLLADPKFA
QFSQEIGLASLGASDEDVQKLATCYFFTIEFGLCKQEGQLRAYGAGL
LSSIGELKHALSDKACVKAFDPKTTCLQECLITTFQEAYFVSESFEE
AKEKMRDFAKSITRPFSVYFNPYTQSIEILKDT*
TPH (Schistosoma mansoni) (Example 7):
(SEQ ID NO: 177)
MISTESDLRRQLDENVRSEADESTKEECPYINAVQSHHQNVQEMSII
ISLVKNMNDMKSIISIFTDRNINILHIESRLGRLNMKKHTEKSEFEP
LELLVHVEVPCIEVERLLEELKSFSSYRIVQNPLMNLPEAKNPTLDD
KVPWFPRHISDLDKVSNSVLMYGKELDADHPGFKDKEYRKRRMMFAD
IALNYKWGQQIPIVEYTEIEKTTWGRIYRELTRLYKTSACHEFQKNL
GLLQDKAGYNEFDLPQLQVVSDFLKARTGFCLRPVAGYLSARDFLSG
LAFRVFYCTQYIRHQADPFYTPEPDCCHELLGHVPMLADPKFARFSQ
EIGLASLGTSDEEIKKLATCYFFTIEFGLCRQDNQLKAYGAGLLSSV
AELQHALSDKAVIKPFIPMKVINEECLVTTFQNGYFETSSFEDATRQ
MREFVRTIKRPFDVHYNPYTQSIEIIKTPKSVAKLVQDLQFELTAIN
ESLLKMNKEIRSQQFTTNKIVTENRSS*
PTS (Rattus norvegicus) (Example 1, 2 and 7):
(SEQ ID NO: 18)
MNAAVGLRRRARLSRLVSFSASHRLHSPSLSAEENLKVFGKCNNPNG
HGHNYKVVVTIHGEIDPVTGMVMNLTDLKEYMEEAIMKPLDHKNLDL
DVPYFADVVSTTENVAVYIWENLQRLLPVGALYKVKVYETDNNIVVY
KGE*
SPR (Rattus norvegicus) (Example 1, 2 and 7):
(SEQ ID NO: 24)
MEGGRLGCAVCVLTGASRGFGRALAPQLAGLLSPGSVLLLSARSDSM
LRQLKEELCTQQPGLQVVLAAADLGTESGVQQLLSAVRELPRPERLQ
RLLLINNAGTLGDVSKGFLNINDLAEVNNYWALNLTSMLCLTTGTLN
AFSNSPGLSKTVVNISSLCALQPFKGWGLYCAGKAARDMLYQVLAVE
EPSVRVLSYAPGPLDTNMQQLARETSMDPELRSRLQKLNSEGELVDC
GTSAQKLLSLLQRDTFQSGAHVDFYDI*
DHPR (Escherichia coli) (Example 2):
(SEQ ID NO: 39)
MDIISVALKRHSTKAFDASKKLTPEQAEQIKTLLQYSPSSTNSQPWH
FIVASTEEGKARVAKSAAGNYVFNERKMLDASHVVVFCAKTAMDDVW
LKLVVDQEDADGRFATPEAKAANDKGRKFFADMHRKDLHDDAEWMAK
QVYLNVGNFLLGVAALGLDAVPIEGFDAAILDAEFGLKEKGYTSLVV
VPVGHHSVEDFNATLPKSRLPQNITLTEV*
DHPR (Homo sapiens) (Example 1 and 7):
(SEQ ID NO: 35)
MAAAAAAGEARRVLVYGGRGALGSRCVQAFRARNWWVASVDVVENEE
ASASIIVKMTDSFTEQADQVTAEVGKLLGEEKVDAILCVAGGWAGGN
AKSKSLFKNCDLMWKQSIWTSTISSHLATKHLKEGGLLTLAGAKAAL
DGTPGMIGYGMAKGAVHQLCQSLAGKNSGMPPGAAAIAVLPVTLDTP
MNRKSMPEADFSSWTPLEFLVETFHDWITGKNRPSSGSLIQVVTTEG
RTELTPAYF*
PCBD1 (Homo sapiens) (Example 1):
(SEQ ID NO: 34)
MAGKAHRLSAEERDQLLPNLRAVGWNELEGRDAIFKQFHFKDFNRAF
GFMTRVALQAEKLDHHPEWFNVYNKVHITLSTHECAGLSERDINLAS
FIEQVAVSMT*
PCBD1 (Lactobacillus reuteri)
(Example 1 and 7):
(SEQ ID NO: 32)
MVKLFPSENARRWHRWNHEVLLLVNIQCSLKQPLWSAEGKVDKNREK
CAAFVYRLVEIQDARI*
DDC (Homo sapiens) (Example 1, 2 and 7):
(SEQ ID NO: 44)
MNASEFRRRGKEMVDYMANYMEGIEGRQVYPDVEPGYLRPLIPAAAP
QEPDTFEDIINDVEKIIMPGVTHWHSPYFFAYFPTASSYPAMLADML
CGAIGCIGFSWAASPACTELETVMMDWLGKMLELPKAFLNEKAGEGG
GVIQGSASEATLVALLAARTKVIHRLQAASPELTQAAIMEKLVAYSS
DQAHSSVERAGLIGGVKLKAIPSDGNFAMRASALQEALERDKAAGLI
PFFMVATLGTTTCCSFDNLLEVGPICNKEDIWLHVDAAYAGSAFICP
EFRHLLNGVEFADSFNFNPHKWLLVNFDCSAMWVKKRTDLTGAFRLD
PTYLKHSHQDSGLITDYRHWQIPLGRRFRSLKMWFVFRMYGVKGLQA
YIRKHVQLSHEFESLVRQDPRFEICVEVILGLVCFRLKGSNKVNEAL
LQRINSAKKIHLVPCHLRDKFVLRFAICSRTVESAHVQRAWEHIKEL
AADVLRAERE*
AANAT-A55P (Bos taurus) (Example 1 and 7):
(SEQ ID NO: 178)
MSTPSIHCLKPSPLHLPSGIPGSPGRQRRHTLPANEFRCLTPEDAAG
VFEIEREPFISVSGNCPLNLDEVRHFLTLCPELSLGWFVEGRLVAFI
IGSLWDEERLTQESLTLHRPGGRTAHLHALAVHHSFRQQGKGSVLLW
RYLQHAGGQPAVRRAVLMCEDALVPFYQRFGFHPAGPCAVVVGSLTF
TEMHCSLRGHAALRRNSDR
ASMT (Gallus gallus) (Example 2):
(SEQ ID NO: 62)
MDSTEDLDYPQIIFQYSNGFLVSKVMFTACELGVFDLLLQSGRPLSL
DVIAARLGTSIMGMERLLDACVGLKLLAVELRREGAFYRNTEISNIY
LTKSSPKSQYHIMMYYSNTVYLCWHYLTDAVREGRNQYERAFGISSK
DLFGARYRSEEEMLKFLAGQNSIWSICGRDVLTAFDLSPFTQIYDLG
GGGGALAQECVFLYPNCTVTIYDLPKVVQVAKERLVPPEERRIAFHE
GDFFKDSIPEADLYILSKILHDWDDKKCRQLLAEVYKACRPGGGVLL
VESLLSEDRSGPVETQLYSLNMLVQTEGKERTAVEYSELLGAAGFRE
VQVRRTGKLYDAVLGRK*
ASMT (Homo sapiens) (Example 1 and 7):
(SEQ ID NO: 59)
MGSSEDQAYRLLNDYANGFMVSQVLFAACELGVFDLLAEAPGPLDVA
AVAAGVRASAHGTELLLDICVSLKLLKVETRGGKAFYRNTELSSDYL
TTVSPTSQCSMLKYMGRTSYRCWGHLADAVREGRNQYLETFGVPAEE
LFTAIYRSEGERLQFMQALQEVWSVNGRSVLTAFDLSVFPLMCDLGG
GAGALAKECMSLYPGCKITVFDIPEVVWTAKQHFSFQEEEQIDFQEG
DFFKDPLPEADLYILARVLHDWADGKCSHLLERIYHTCKPGGGILVI
ESLLDEDRRGPLLTQLYSLNMLVQTEGQERTPTHYHMLLSSAGFRDF
QFKKTGAIYDAILARK*
GCH1 (Escherichia coli) (Example 2):
(SEQ ID NO: 12)
MPSLSKEAALVHEALVARGLETPLRPPVHEMDNETRKSLIAGHMTEI
MQLLNLDLADDSLMETPHRIAKMYVDEIFSGLDYANFPKITLIENKM
KVDEMVTVRDITLTSTCEHHFVTIDGKATVAYIPKDSVIGLSKINRI
VQFFAQRPQVQERLTQQILIALQTLLGTNNVAVSIDAVHYCVKARGI
RDATSATTTTSLGGLFKSSQNTRHEFLRAVRHHN*
Gene sequences are shown in SEQ ID NO: 150
(PTS codon optimized for S. cerevisiae);
151 (SPR codon optimized for S. cerevisiae);
152 (DHPR codon optimized for S. cerevisiae);
153 (PCBD1 codon optimized for S. cerevisiae);
154 (TPH codon optimized for E. coli);
155 (PTScodon optimized for E. coli);
156 (SPR codon optimized for E. coli);
157 (DHPR codon optimized for E. coli);
158 (PCBD1 codon optimized for E. coli);
159 (DDC codon optimized for E. coli);
160 (AANAT codon optimized for E. coli);
161 (ASMT codon optimized for E. coli),
179 (H. sapiens TPH, truncated 146-460);
180 (S. mansoni TPH);
181 (H. sapiens DHPR);
182 (L. reuteri PCBD1);
183 (B. taurus AANAT-A55P);
184 (H. sapiens ASMT).

### Example 1

This Example describes the reconstruction of exogenous pathways for 5HTP or melatonin production in Saccharomyces cerevisiae.

Laboratory S. cerevisiae strain CEN.PK113-7D (MATa URA3 HIS3 LEU2 TRP1 MAL2-8^{C} SUC2) was used as reference strain, and the S. cerevisiae strain CEN.PK102-5B (MATa ura3-52 his3Δ1 leu2-3/112 TRP1 MAL2-8^{C} SUC2) was used for strain construction.

Genes encoding a TPH, the THB synthesis pathway including a PTS and an SPR, and the THB recycling pathway including a PCBD1 and a DHPR, were synthesized and incorporated into integration plasmids pX-3-KILEU2, pXII-5-SpHIS5/pX-4-SpHIS5, and pXI-3-KIURA3, respectively, using the method developed by Mikkelsen et al. (2012). The primers used for the cloning are listed in Table 2. The TPH, PTS, SPR, PCBD1, DHPR genes were all expressed under strong promoters, TEF1, PGK1, PGK1, TEF1, and TEF1, respectively. The constructed insertion plasmids were transformed into a S. cerevisiae CEN.PK102-5B strain following the lithium acetate/single-stranded carrier DNA/PEG method (Gietz and Schiestl, 2007). The TPH gene was integrated onto the chromosome XI at site No. 3. The PTS and DHPR genes were integrated onto either the chromosome XII at site No. 5 or the chromosome X at site No. 4, and the SPR and PCBD1 genes were integrated onto the chromosome X at site No. 3 (Mikkelsen et al., supra).

The derived strain ST783 was tested for 5HTP production. A Delft medium supplemented with 20 g/L of glucose as a carbon source for cell growth, and 2 g/L of tryptophan was supplemented as a substrate for the pathway. The culture was incubated at 30° C. and 250 rpm for 72 hours. The culture broth was collected and centrifuged. The supernatant was collected and filtered for HPLC analysis. The mobile phase of the HPLC measurement was 80% 10 mM NH₄COOH adjusted to pH 3.0 with HCOOH and 20% acetonitrile. The flow rate was set at 0.5 ml/min. A Discovery HS F5 column (Sigma) was used for the separation. UV detection at 254 nm and a fluorescent detector with excitation at 315 nm and emission at 335 nm was used for detection of 5HTP, serotonin, N-acetylserotonin, and melatonin.

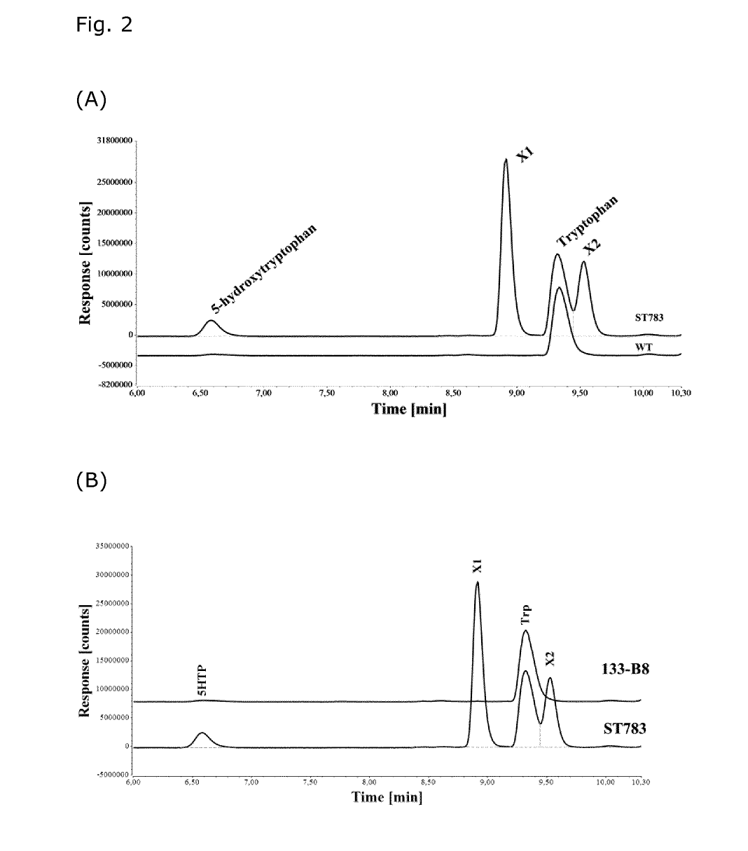

The cells of strain ST783 produced about 1.0 mg/L of 5HTP in microtiter plate-based small-scale cultivations. Two other major peaks were also found in the chromatograph, suspected of being 5HTP degradation products (X1 and X2) (FIG. 2a).

Another S. cerevisiae CEN.PK102-5B based strain containing the gene encoding a TPH but not the pathways for THB synthesis and regeneration was also constructed, and tested for 5HTP production in comparison to the ST783 strain. As FIG. 2b shows, the S. cerevisiae 133-B8 strain did not show 5HTP production, and no 5HTP degradation product was found on the spectrum. This evidenced that the THB synthesis pathway was necessary, and the THB regeneration pathway should be beneficial, for 5HTP synthesis in the experimental strain.

Genes coding DDC, AANAT, and ASMT were synthesized and incorporated onto two integrative plasmids pXII-1-KILEU2 and pXI-5-LoxP-SpHIS5 using the method developed by Mikkelsen et al. (supra). The DDC, AANAT, and ASMT genes were expressed under strong promoters, TEF1, PGK1, and TEF1 promoters, respectively. The TEF1 and PGK1 promoter DNA was amplified using the primers TEF1-FWD with TEF1-REV, and PGK1-FWD with PGK1-REV, respectively using genomic DNA of S. cerevisiae as the template for PCR reactions. The resulting DNA pieces were fused using the USER cloning method for the construction of insertion plasmids (Nour-Eldin et al., 2006).

The constructed insertion plasmids were transformed into a S. cerevisiae CEN.PK102-5B strain following the lithium acetate/single-stranded carrier DNA/PEG method (Gietz and Schiestl, supra). The DDC and AANAT genes were integrated onto the chromosome XI on site No. 5, and the ASMT gene was integrated onto the chromosome XII site No. 1 (Mikkelsen et al., supra).

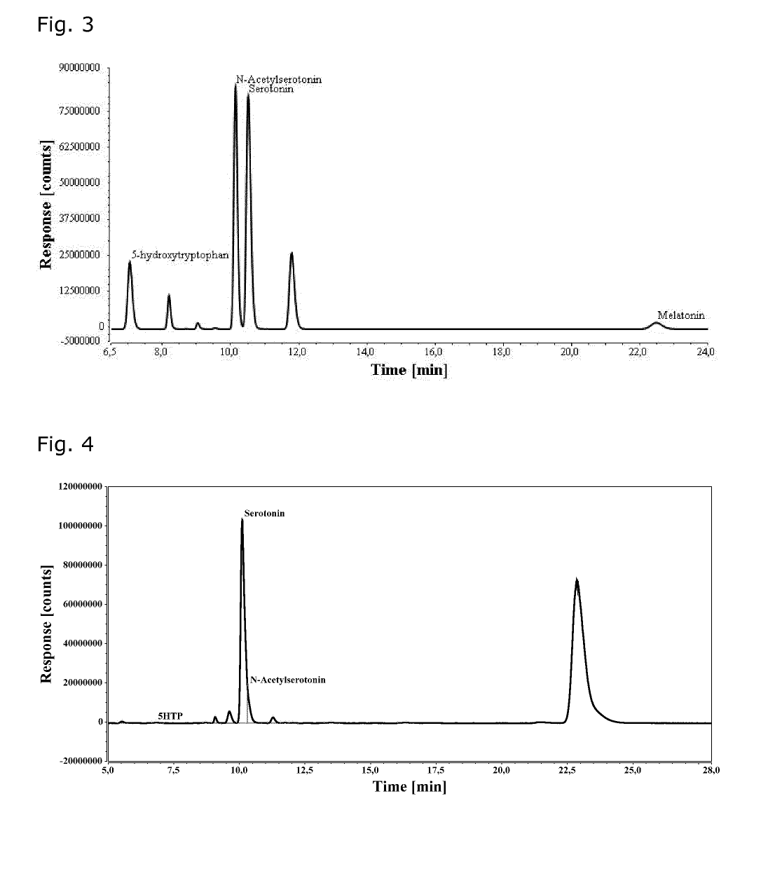

The derived strain ST892 was tested for melatonin production. A Delft medium supplemented with 20 g/L of glucose as a carbon source for cell growth, and 100 mg/L of 5HTP was supplemented as a substrate for the pathway. The cell produced about 4.8 mg/L of melatonin, 36.1 mg/L of serotonin, and 30.7 mg/L of N-acetylserotonin (FIG. 3) after 72 hours of culturing in microtiter plate-based small-scale cultivations, which showed that the integrated DDC, AANAT, and ASMT enzymes were functional in the cell.

The selection markers on strain ST892 were removed by transforming a plasmid carrying a cre recombinase (pGAL1-cre (SEQ ID NO:145). The transformant cells were grown on Yeast extract-Peptone-Galactose (YPG) medium for 8-12 hours and then selected based on the cell growth on culture plates without leucine, uracil, or histidine. Clony PCR check (data not shown) of the resulted strain ST916 showed that the ASMT gene was lost during the recombination process, but the strain retained the AANAT and DDC gene.

The ST916 strain was transformed with the integration plasmids containing genes encoding above mentioned pathway genes for 5HTP production, such as TPH, PTS, SPR, PCBD1, and DHPR. The derived strain S. cerevisiae ST925 was grown on Delft medium supplemented with 2% glucose as a carbon source, and 2 g/L of tryptophan as a substrate for the pathway.

The cell produced small amount of 5HTP, ca. 28.4 mg/L of serotonin and a small amount of N-acetylserotonin after 72 hours of culture in shake flasks (FIG. 4).

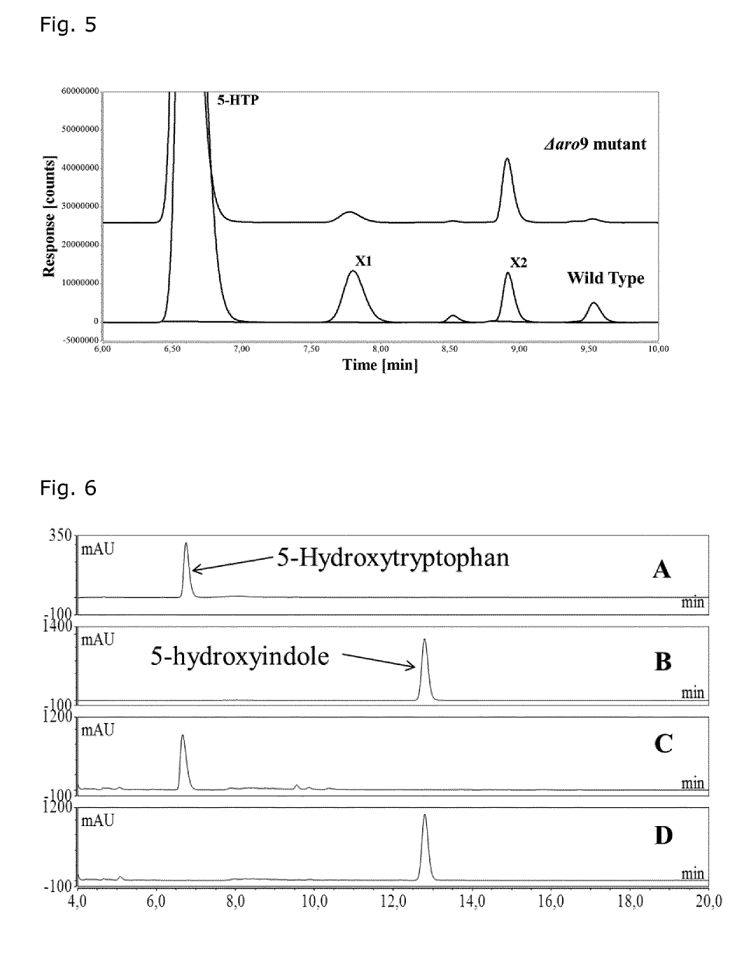

The deletion of the aro9 gene from the S. cerevisiae strain reduced the degradation of 5HTP. A S. cerevisiae Δaro9 mutant strain S. cerevisiae BY4741 MATa his3Δ1 leu2Δ0 met15Δ0 ura3Δ0 aro9Δ0 ordered from openbiosystems (YKO MATa Strain Collection) was cultivated in microtiter plate on Delft medium supplemented with 5HTP. As compared to the wild type S. cerevisiae strain, the degradation of 5-hydroxytryptophan into compound X1 was significantly reduced in the culture of the Δaro9 mutant strain as compared to the control strain S. cerevisiae CEN.PK113-7D (MATa URA3 HIS3 LEU2 TRP1 MAL2-8^{C} SUC2) (FIG. 5).

Since the degradation products X1 and X2 peaks disappeared in the cultures of strain ST892, which contains DDC, AANAT, and ASMT besides TPH, the DDC reaction was efficient enough to compete with the 5HTP degradation pathways and redirected the flux towards serotonin production.

### Example 2

This Example describes the reconstruction of exogenous pathways for 5HTP and melatonin production in Escherichia coli.

Genes encoding a TPH, the THB synthesis pathway including a GCH1, a PTS and an SPR, and the THB regeneration pathway including a PCBD1 and a DHPR were synthesized and incorporated into two compatible plasmids. The genes encoding TPH, PCBD1, and DHPR were organized as one operon (TDP operon; SEQ ID NO:146) and incorporated on the pUC18 to derive the pTDP plasmid. The genes were expressed under a lac promoter. The genes encoding GCH1, PTS, and SPR were organized as one operon (GPS operon; SEQ ID NO:147) and incorporated on the pTH19cr for the construction of the pTHB plasmid. The genes on GPS operon were expressed under a lac promoter. The plasmids were constructed using a DNA assembling method (Gibson et al., 2009).

The wild type E. coli MG1655 strain can degrade 5HTP into 5-hydroxyindole. The enzyme catalysing this reaction in E. coli is tryptophanase. While not being limited to theory, this is mostly due to the similarity between the chemical structures of tryptophan and 5HTP.

By knocking out the tnaA gene, which encodes tryptophanase in E. coli, using a standard gene knockout protocol (Datsenko and Wanner, 2000), the degradation of 5HTP was eliminated (FIG. 6) in the culture of the E. coli MG1655 ΔtnaA::FRT mutant strain (MGT). The primers used for gene knockout (H1-P1-tnaA and H2-P2-tnaA) and confirmation (K1 and CFB-FWD-tnaA) are listed in Table 2.

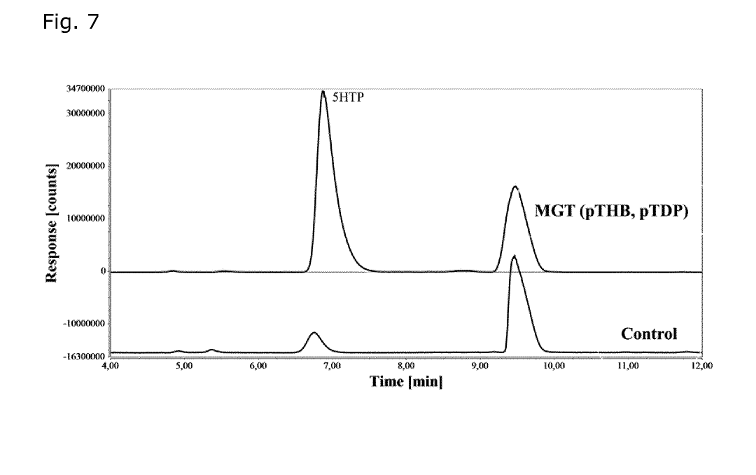

The constructed pTDP and pTHB plasmids were co-transformed into the E. coli MGT (E. coli MG1655 ΔtnaA::FRT mutant) strain. The transformant strain was cultured in LB medium supplemented with 5% of glycerol and 2 g/L of tryptophan. Samples were collected after 24 hours of culture at 30° C. and analyzed on HPLC (FIG. 7).

### Example 3

This Example describes improving the production of melatonin in S. cerevisiae by enriching AcCoA in the cell.

Acetyl coenzyme A (AcCoA) serves as a metabolic cofactor in the serotonin acetyltransferase reaction. The conversion of serotonin into N-acetylserotonin was not complete in the previously constructed strains (see FIG. 3 and FIG. 4). Engineering the AcCoA metabolism can thereby improve the supply of the precursor for the reaction. A higher AcCoA/CoA ratio changes the thermodynamic equilibrium of the reaction, making it more favorable towards N-Acetetylserotonin generation. Feeding carbon sources such as glucose during the culture improves AcCoA availability in the cell, but there are many competing pathways using AcCoA as a substrate. It has been shown, however, that engineering the metabolic pathways can better improve the supply of AcCoA (Kocharin et al., 2012). Therefore, the following pathway modification approach was designed to facilitate the ASMT reaction:

A cytosolic acetaldehyde dehydrogenase (EC 1.2.1.4) and an acetyl coenzyme A synthetase (EC 6.2.1.1) are recombinantly introduced in order to convert the acetaldehyde into AcCoA. The acetyl-CoA synthase gene is from Salmonella enterica (Shiba et al., 2007) and the gene is amplified using the primers ACS1-FWD and ACS1-Rev (Table 2). The ALD6 gene is amplified using primers ALD6-FWD and ALD6-Rev (Table 51). The genes are expressed under TEF1 and PGK1 promoters, respectively. The plasmid derived is named p0380 (SEQ ID NO:148) and plasmid p0380 is transformed into a melatonin producing S. cerevisiae strain to improve melatonin production.

### Example 4

This Example described improving the production of melatonin in S. cerevisiae by enriching S-adenosylmethionine (SAM) in the cell.

S-adenosylmethionine (SAM) is a principal methyl donor in various intracellular transmethylation reactions. It is synthesized in the cell through SAM synthetase from methionine and ATP. In the melatonin production pathway, SAM is used as a substrate in the ASMT reaction for synthesis of melatonin. Enriching SAM in the cell will shift the reaction equilibrium towards the generation of melatonin, thereby improving the productivity of melatonin.

SAM can be directly supplemented in the culture medium in order to improve the metabolic flux through the ASMT reaction. However, it is rather costly and not applicable in industrial scale productions. As an alternative, the precursor of SAM, methionine, can be supplemented in the culture medium. The supplemented methionine could be converted into SAM through SAM synthetase and thereby enrich SAM in the cell. However, in a preliminary study, the data (not shown) indicated that melatonin production in strain ST892 was not improved as compared with the control, which was grown on a medium without methionine supplementation. Therefore, several genetic modification strategies can be used for the enrichment of SAM in the cell based on pathway analysis and previous studies. The following pathway modification approaches were designed:

(A) Overexpressing the SAM synthetase. This is an immediate option for enriching SAM supply. There are two isogenes, SAM1 and SAM2, in S. cerevisiae encoding SAM synthetase. The SAM2 is chosen for overexpression since it has been shown that the expression of SAM2 gene increases during cell growth, and overrides the repression effect of SAM (Thomas and Surdin-Kerjan, 1991). Combining the overexpression of SAM2 and supplementing methionine in the culture medium improves the SAM supply for the ASMT reaction. The SAM2 gene is amplified using the primers SAM2-fw and SAM2-rev using genomic DNA of S. cerevisiae as the template. The amplified gene is expressed under TEF1 promoter, and the operon integrated onto chromosome as previously described.

(B) The supplementation of methionine can also cause some cost problem and feedback inhibiting metabolic process in the cell. As an additional or alternative strategy, the whole SAM cycle can be therefore strengthened by overexpressing S-adenosylhomocysteine hydrolase (SAH1) and methionine synthases (Met6) together with SAM synthetase (SAM2) to increase the turnover flux of the SAM cycle, thereby facilitating the production of melatonin through ASMT. The SAM2, SAMH, and MS genes are amplified using the primers SAM2-fw, SAM2-rev, SAH1-FWD, SAH1-REV, Met6-FWD and Met6-REV, respectively. The genes are expressed under TEF1 or PGK1 promoter, and the operons integrated onto chromosome as previously described.

(C) It has been reported that overexpressing the ethionine resistance protein ERC1 improved the accumulation of SAM in a yeast strain (Lee et al., 2010). For the purpose of improving SAM accumulation, the SAM2 and ERC1 genes are amplified using the primers SAM2-fw, SAM2-rev, ERC1-fwd, and ERC1-rev, respectively. The two genes are expressed under TEF1 and PGK1 promoter, respectively, and then incorporated onto chromosome as previously described to supply SAM for the ASMT reaction towards melatonin production.

### Example 5

This Example describes increasing tryptophan production for the purposes 5HTP and melatonin production from simple carbon sources.

Tryptophan is the precursor of 5HTP and melatonin pathway in the current invention. Optionally, tryptophan can supplemented into the culture medium, and another carbon source such as glucose or glycerol added in order to generate energy or metabolic cofactors for the 5HTP or melatonin pathway. Avoiding tryptophan supplementation, however, can not only reduce the cost of the process, but also eliminates the physiological effects of tryptophan on the cell such as feedback inhibitions. In order to do that, metabolic engineering can modify the metabolic network to generate sufficient tryptophan, e.g. to achieve a higher productivity of the desired compound(s). The following pathway modification approaches were designed:

(A) The trpR gene is knocked-out in the E. coli MGT strain in order to remove the transcriptional regulation of the TrpR-tryptophan complex on the expression of trpABCDE and aroH genes. The primers for deleting the trpR gene (H1-P1-trpR and H2-P2-trpR) and the primers used for the confirmation of gene knockout (K1 and trpR-cfm) are listed in Table 2.

(B) Tryptophan is one of the aromatic amino acids synthesized through the shikimate pathway. The shikimate pathway is under complicated metabolic regulations. One of the major feedback regulation steps is the 3-deoxy-D-arabino-heptulosonate 7-phosphate (DAHP) synthase reaction. Releasing the feedback regulation on this step resulted at significant accumulation of aromatic amino acids, including tryptophan (Patnaik et al., 1995). A feedback resistant version of DAHP synthase (AroG-fbr) has been reported to boost up tryptophan production in E. coli (Patnaik et al., 1995). Therefore, the AroG-fbr protein is expressed by incorporating the gene on a pK18 plasmid (SEQ ID NO:149) under an inducible plac promoter. Primers for assembling the genes are listed in Table 2 (pK18-FWD, pK18-Rev, aroG-Fwd, aroG-Rev).

(C) The synthesis of shikimic acid uses phosphoenolpyruvate (PEP) and erythrose 4-phosphate (E4P) as the precursors. Overexpressing transketolase and PEP synthase in E. coli enriches the metabolic pool of these compounds (Patnaik et al., supra). The tktA and ppsA genes are therefore incorporated together with the aroG-fbr gene using the DNA assembly method. Primers for the assembly, including pAroG-Fwd, pAroG-Rev, tktA-fwd, tktA-r ev, ppsA-fwd, and ppsA-rev, are listed in Table 2.

(D) The supply of tryptophan for melatonin production can also be improved by disrupting the phosphotransferase (PTS) system in, e.g., E. coli. PTS is a group translocation system transports sugars including glucose into the cell. The system uses one mole of PEP for the transport and phosphorylation of one mole of glucose. Therefore, disrupting the PTS by knocking out the ptsH gene (using primers H1-P1-ptsH and H2-P2-ptsH) reserves PEP for the synthesis of shikimic acid, and thereafter tryptophan for the melatonin synthesis.

### Example 6

This Example describes the production of 5HTP and melatonin from simple carbon sources. Simple carbon sources such as glucose and glycerol can be readily consumed by either S. cerevisiae or E. coli wild type strains. The carbon source can be catabolized into precursor molecules for biomass synthesis including tryptophan, the substrate of the melatonin pathway. Incorporating the exogenous 5HTP or melatonin pathway in the cell according to the invention can redirect the metabolic flux distribution toward the production of 5HTP and melatonin. For this purpose, TPH as well as the genes in the THB synthesis and regeneration pathways are required for the production of 5HTP, and increasing SAM production and/or regeneration can further improve the productivity of melatonin, optionally in combination with other approaches described herein.

A fed batch culture process is used for the production of 5HTP and melatonin. The optimized strain made as described in any one of the previous examples is cultured using a synthetic medium with supplementation of glucose or glycerol. A 250 ml shaking flask filled with 50 ml of synthetic medium with 5 g/L of glucose is inoculated with 50 μl of overnight pre-culture. The microtiter plate-based small-scale cultivation is incubated at 30° C. and 300 rpm. Another 5 g/L of glucose is added after the culture reach stationary phase, and a third batch of glucose is added after the previous batch of carbon source is used up.

### Example 7

This Example describes the reconstruction of exogenous pathways for melatonin production in Saccharomyces cerevisiae resulting in melatonin production from glucose.

Laboratory S. cerevisiae strain CEN.PK113-7D (MATa URA3 HIS3 LEU2 TRP1 MAL2-8^{C} SUC2) was used as reference strain, and the S. cerevisiae strain CEN.PK102-5B (MATa ura3-52 his3Delta1 leu2-3/112 TRP1 MAL2-8^{C} SUC2) was used for strain construction.

Genes encoding H. sapiens TPH (45-471(+20)), the THB synthesis pathway including R. norvegicus PTS and R. norvegicus SPR, and the THB recycling pathway including L. ruminis PCBD1 and H. sapiens DHPR, were synthesized and incorporated into integration plasmids pXI-3-KIURA3, pX-3-KILEU2, and pX-4-SpHIS5, respectively, using the method developed by Mikkelsen et al. (2012). The primers used for the cloning are listed in Table 2. The TPH, PTS, SPR, PCBD1, DHPR genes were all expressed under strong promoters, PGK1, TEF1, PGK1, TEF1, and PGK1, respectively. The promoters were amplified using genomic DNA of S. cerevisiae as the template for PCR reactions. The primers used for the cloning are listed in Table 2. The resulting DNA fragments were assembled using the USER cloning method for the construction of insertion plasmids (Nour-Eldin et al., 2006).

The constructed insertion plasmids linearized by NotI were transformed into the CEN.PK102-5B strain via the lithium acetate/single-stranded carrier DNA/PEG method (Gietz and Schiestl, 2007). The TPH (45-471(+20)) gene was integrated into the chromosome XI at site No. 3. The PTS and SPR genes were integrated into the chromosome X at site No. 3, and the DHPR and PCBD1 genes were integrated onto the chromosome X at site No. 4 (Mikkelsen et al., supra). Correct integration at the specific genomic loci was verified by PCR.

The derived strain SCE-iL1-108 was transformed with a plasmid carrying a cre recombinase (pGAL1-cre, KanMX (SEQ ID NO:145) to remove the auxotrophic selection markers. The transformed cells were grown in Yeast extract-Peptone-Galactose (YPG) medium supplemented with 200 μg/ml G418 (Invitrogen) in a shaking incubator for 16 hours at 30° C. The cells were centrifuged, the supernatant removed, and the cell pellet was resuspended in YPG. After another 7.5 hours at 30° C. in a shaking incubator, the cells were pelleted and the supernatant removed, and a dilution series plated on Yeast extract-Peptone-Glucose (YPD). Clones with looped out markers were selected based on cell growth on SC media plates lacking leucine, uracil, or histidine, and loss of plasmid was confirmed on YPD+G418 plates, resulting in strain SCE-iL1-120.

This S. cerevisiae strain containing the 5HTP pathway with looped out markers (SCE-iL1-120) was transformed with the pathway genes for producing melatonin from 5HTP, such as H. sapiens DDC, B. taurus AANAT-A55P, and H. sapiens ASMT. These genes were synthesized and incorporated onto two integrative plasmids pXI-5-LoxP-SpHIS5 and pXII-1-KILEU2 using the method developed by Mikkelsen et al. (supra). The DDC, AANAT-A55P, and ASMT genes were expressed under strong promoters, TEF1, PGK1, and TEF1 promoters, respectively. The promoters were amplified using genomic DNA of S. cerevisiae as the template for PCR reactions. The primers used for the cloning are listed in Table 2. The resulting DNA pieces were fused using the USER cloning method for the construction of insertion plasmids (Nour-Eldin et al., 2006).

The constructed insertion plasmids were linearized by NotI and transformed into the S. cerevisiae SCE-iL1-120 strain via the lithium acetate/single-stranded carrier DNA/PEG method (Gietz and Schiestl, supra). The DDC and AANAT-A55P genes were integrated into chromosome XI site No. 5, and the K. lactis URA3 marker was integrated into chromosome X site No. 2, resulting in strain SCE-iL1-138. The ASMT gene was integrated into chromosome XII site No. 1 (Mikkelsen et al., supra), resulting in strain SCE-iL1-139. Correct integration at the specific genomic loci was verified by PCR.

Finally, the genes coding S. mansoni TPH and H. sapiens TPH(146-460) were synthesized and incorporated into the integrative plasmid pTy1-LoxP-KanMXsyn using the method developed by Mikkelsen et al. (supra). The TPH genes were expressed under the strong PGK1 promoter. The promoter was amplified using genomic DNA of S. cerevisiae as the template for PCR reactions. The primers used for the cloning are listed in Table 2. The resulting DNA pieces were fused using the USER cloning method for the construction of insertion plasmids (Nour-Eldin et al., 2006).

The constructed insertion plasmids were linearized by NotI and transformed into the S. cerevisiae SCE-iL1-139 strain via the lithium acetate/single-stranded carrier DNA/PEG method (Gietz and Schiestl, supra). The S. mansoni TPH and H. sapiens TPH(146-460) genes were integrated into chromosomal Ty1 retrotransposon sites, resulting in strains SCE-iL3-HM-26 and SCE-iL3-HM-27, respectively.

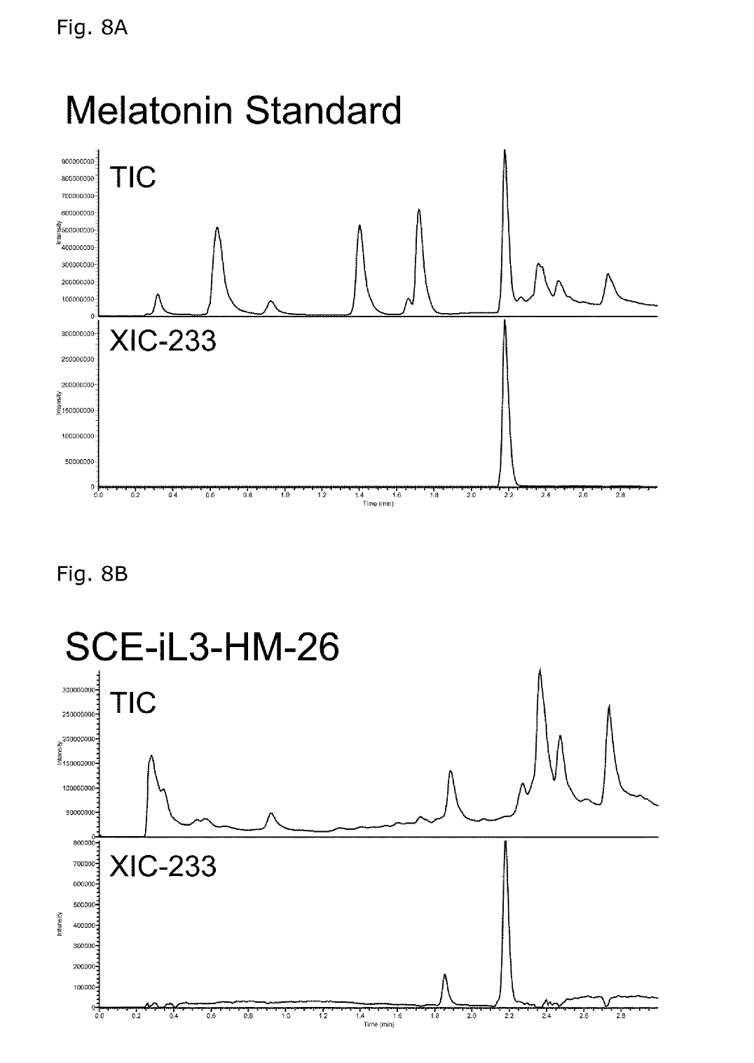

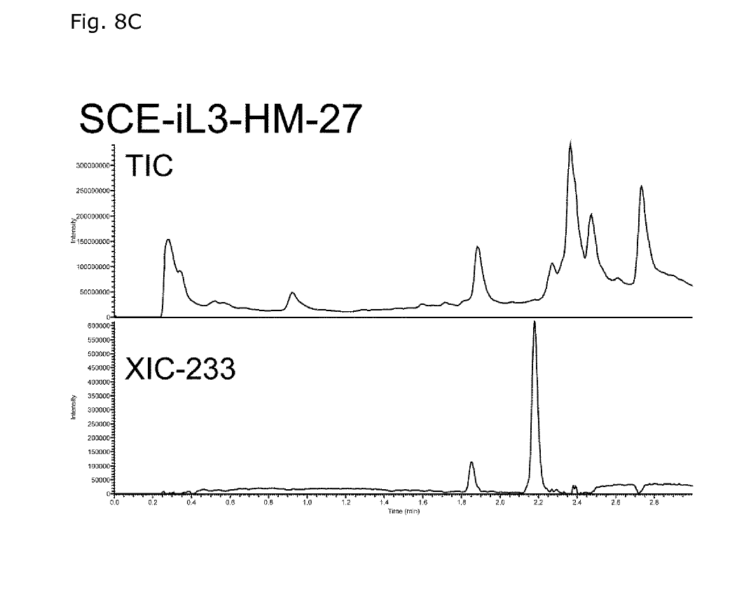

The derived strains SCE-iL3-HM-26 and SCE-iL3-HM-27 were tested for melatonin production. Experiments were carried out in 96-deep-well plates by applying 400 μl Delft medium supplemented with 20 g/l glucose at 30° C. at 250 rpm. Cells were allowed to grow for 72 h. The supernatant was collected, filtered (0.22 μm) and subjected to LC-MS analysis and melatonin at 86.8±7.0 μg/l and 66.5±1.9 μg/L was produced from SCE-iL3-HM-26 and SCE-iL3-HM-27, respectively (FIGS. 8B and 8C).

LC-MS data was collected on OrbiTrap Fusion High Resolution Mass Spectrometer system coupled with an Ultimate 3000 UHPLC pump (Thermo, San Jose Ca). Samples were held in the autosampler at a temperature of 10.0° C. during the analysis. 1 uL Injections of the sample were made onto a Thermo HyperSil Gold PFP HPLC column, with a 3 um particle size, 2.1 mm i.d. and 150 mm long. The column was held at a temperature of 35.0° C. The solvent system used was Solvent A “Water with 0.1% formic acid” and Solvent B “Acetonitrile with 0.1% formic”. The Flow Rate was 1.000 ml/min with an Initial Solvent composition of % A=95, % B=5 held until 0.50 min, the solvent composition was then changed following a Linear Gradient until it reached % A=70.0 and % B=30.0 at 1.50 min. The solvent composition was then changed following a Linear Gradient until it reached % A=5.0 and % B=95.0 at 2.00 min This was held until 2.50 min when the solvent was returned to the initial conditions and the column was re-equilibrated until 3.00 min. The first 0.25 min of the run was diverted to waste using the divert valve, following which the column eluent flowed directly into the Heated ESI probe of the MS which was held at 325° C. and a voltage of 3500 V. Data was collected in positive ion mode over the mass range 50 to 1000 m/z at a resolution of 15,000. The other MS settings were as follows, Sheath Gas Flow Rate of 60 units, Cone Gas Flow Rate of 20 units Cone Temp was 275° C.

In conclusion, we have established melatonin production from glucose as a sole carbon source without the addition of tryptophan. TPH from S. mansoni and H. sapiens have been identified as promising candidates for the production of melatonin. When using TPH from S. mansoni, higher titres were achieved (app. 30%).

Each and every publication referred to herein is hereby incorporated by reference, in its entirety.

The terms used herein is intended to be used to describe the embodiments, not to limit the present invention. Terms without numbers in front are not to limit the quantity but to show that there may be more than one thing of the term used. The term “including”, “having”, “consisting”, and “comprising” shall be interpreted openly (i.e. “including but not limited to”).

Although the present invention is described and shown by exemplary embodiments and Examples, those skilled in the art will understand well that there can be various changes in the form and details without departing from the spirit of the invention and range defined by the claims. Thus, the present invention, as allowed by the patent law, includes equivalents, and variations thereof, of the key points of the invention stated in the appended claims.

### LIST OF REFERENCES

Crabtree and Channon, Nitric Oxide 2011, 25, 81-88.
Datsenko and Wanner, PNAS 2000, 97, 6640-6645.
Gibson et al., Nature methods 2009, 6, 343-345.
Gietz and Schiestl, Nat Protoc 2007, 2, 38-41.
Kocharin et al., AMB Express 2012, 2, 52.
Lee et al., Korean 3. Chem. Eng. 2010, 27, 587-589.
Meadow et al., Annu Rev Biochem 1990, 59, 497-542.
Mikkelsen et al., Metabolic Engineering 2012, 14, 104-111.
Nour-Eldin et al., Nucleic Acids Res 2006, 34, e122.
Patnaik et al., Biotechnol Bioeng 1995, 46, 361-370.
Shiba et al., Metabolic Engineering 2007, 9, 160-168.
Thomas and Surdin-Kerjan, Mol Gen Genet 1991, 226, 224-232.
U.S. Pat. No. 7,807,421 B2
WO 2012/135389
Yamamoto et al., E. Kataoka, N. Miyamoto, et al., Metab Eng 2003, 5, 246-254.

We claim:

1. A recombinant microbial cell comprising exogenous nucleic acid sequences encoding an L-tryptophan hydroxylase (TPH) (EC 1.14.16.4), a 5-hydroxy-L-tryptophan decarboxylase (DDC) (EC 4.1.1.28), a serotonin acetyltransferase (AANAT) (EC 2.3.1.87 or EC 2.3.1.5), an acetylserotonin O-methyltransferase (ASMT) (EC 2.1.1.4), and enzymes providing at least one pathway for producing tetrahydrobiopterin (THB), wherein the recombinant microbial cell
(i) further comprises a genetic modification providing for an increase in S-adenosyl-L-methinonine (SAM) production, an increase in acetyl coenzyme A (AcCoA) production, an increase in tryptophan production, or a combination of any thereof; and/or
(ii) comprises an exogenous nucleic acid sequence encoding a TPH which comprises SEQ ID NO:177, SEQ ID NO:176, or functionally active variant, homolog or fragment of any thereof.

2. The recombinant microbial cell of claim 1, wherein the genetic modification in (i) comprises one or more exogenous nucleic acid sequences encoding
(a) a S-adenosylmethionine synthetase (EC 2.5.1.6),
(b) a ethionine resistance protein,
(c) a S-adenosylhomocysteine hydrolase (EC 3.3.1.1),
(d) a methionine synthase (EC 2.1.1),
(e) an AcCoA synthetase (EC 6.2.1.1),
(f) an acetylaldehyde dehydrogenase (EC 1.2.1.3), or
(g) a combination of any two or more of (a) to (f).

3. A recombinant microbial cell comprising exogenous nucleic acid sequences encoding a TPH (EC 1.14.16.4) and enzymes providing at least one pathway for producing THB,
wherein the recombinant microbial cell comprises a genetic modification providing for an increase in tryptophan production.

4. The recombinant microbial cell of claim 1, comprising
(a) a deletion or downregulation of an endogenous gene encoding a tryptophan repressor transcription regulator,
(b) an exogenous nucleic acid sequence encoding a 3-deoxy-d-heptulosonate-7-phosphate (DAHP) synthase (EC 2.5.1.54),
(c) one or more exogenous nucleic acid sequences encoding a transketolase (EC 2.2.1.1) and a PEP synthase (EC 2.7.9.2),
(d) a deletion or downregulation of endogenous genes encoding one or more components of the phosphotransferase system,
(e) one or more exogenous nucleic acid sequences encoding a hexokinase (EC 2.7.1.1) and, optionally, a glucose facilitated diffusion protein (TC 2.A.1.1),
(f) a combination of (a) and (b),
(g) a combination of (c) to (e), or
(h) a combination of any of (a) to (g).

5. The recombinant microbial cell of claim 1, comprising exogenous nucleic acid sequences encoding
(a) a 6-pyruvoyl-tetrahydropterin synthase (PTPS) (EC 4.2.3.12), a sepiapterin reductase (SPR) (EC 1.1.1.153) and, optionally, a GTP cyclohydrolase I (GCH1) (EC 3.5.4.16), and, optionally,
(b) a pterin-4-alpha-carbinolamine dehydratase (PCBD1) (EC 4.2.1.96); and, optionally, a dihydropteridine reductase (DHPR) (EC 1.5.1.34).

6. The recombinant microbial cell of claim 1, comprising
(a) a deletion or downregulation of an endogenous gene encoding an aromatic amino acid aminotransferase (EC 2.6.1.57), and/or
(b) a deletion or downregulation of an endogenous gene encoding a tryptophanase (EC 4.1.99.1).

7. The recombinant microbial cell of claim 1, which is derived from a microbial host cell which is a bacterial cell, a yeast host cell, a filamentous fungal cell, or algal cell.

8. The recombinant microbial cell of claim 1, which is derived from a Saccharomyces, Pichia or Yarrowia cell.

9. The recombinant microbial cell of claim 1, which is derived from a Saccharomyces cerevisiae cell.

10. The recombinant microbial cell of claim 8, which comprises a down-regulation, optionally a deletion, of aro9.

11. The recombinant microbial cell of claim 1, which is derived from an Escherichia, Corynebacteria, Lactobacillus, Bacillus or Pseudomonas cell.

12. The recombinant microbial cell of claim 11, which is derived from an Escherichia coli cell.

13. A method of producing melatonin, comprising culturing a recombinant microbial cell in a medium comprising at least one carbon source and, optionally, isolating melatonin, optionally wherein the medium comprises at least 0.1 g/L methionine and/or at least 0.1 g/L SAM, wherein the recombinant microbial cell comprises:
(a) exogenous nucleic acid sequences encoding an L-tryptophan hydroxylase (TPH) (EC 1.14.16.4), a 5-hydroxy-L-tryptophan decarboxylase (DDC) (EC 4.1.1.28), a serotonin acetyltransferase (AANAT) (EC 2.3.1.87 or EC 2.3.1.5), an acetylserotonin O-methyltransferase (ASMT) (EC 2.1.1.4), and enzymes providing at least one pathway for producing tetrahydrobiopterin (THB), wherein the recombinant microbial cell
(i) further comprises a genetic modification providing for an increase in S-adenosyl-L-methinonine (SAM) production, an increase in acetyl coenzyme A (AcCoA) production, an increase in tryptophan production or a combination of any thereof, and/or
(ii) comprises an exogenous nucleic acid sequence encoding a TPH which comprises SEQ ID NO:177, SEQ ID NO:176, or functionally active variant, homolog or fragment of any thereof, or
(b) exogenous nucleic acid sequences encoding a TPH (EC 1.14.16.4) and enzymes providing at least one pathway for producing THB,
wherein the recombinant microbial cell comprises a genetic modification providing for an increase in tryptophan production.

14. The method of claim 13, wherein the carbon source is selected from the group consisting of glucose, fructose, sucrose, xylose, mannose, galactose, rhamnose, arabinose, fatty acids, glycerol, acetate, starch, glycogen, amylopectin, amylose, cellulose, cellulose acetate, cellulose nitrate, hemicellulose, xylan, glucuronoxylan, arabinoxylan, glucomannan, xyloglucan, lignin, and lignocellulose.

15. The method of claim 14, wherein the carbon source comprises glucose.

16. The recombinant microbial cell of claim 3, comprising
(a) a deletion or downregulation of an endogenous gene encoding a tryptophan repressor transcription regulator,
(b) an exogenous nucleic acid sequence encoding a 3-deoxy-d-heptulosonate-7-phosphate (DAHP) synthase (EC 2.5.1.54),
(c) one or more exogenous nucleic acid sequences encoding a transketolase (EC 2.2.1.1) and a PEP synthase (EC 2.7.9.2),
(d) a deletion or downregulation of endogenous genes encoding one or more components of the phosphotransferase system,
(e) one or more exogenous nucleic acid sequences encoding a hexokinase (EC 2.7.1.1) and, optionally, a glucose facilitated diffusion protein (TC 2.A.1.1),
(f) a combination of (a) and (b),
(g) a combination of (c) to (e), or
(h) a combination of any of (a) to (g).

17. The recombinant microbial cell of claim 3, comprising exogenous nucleic acid sequences encoding
(a) a 6-pyruvoyl-tetrahydropterin synthase (PTPS) (EC 4.2.3.12), a sepiapterin reductase (SPR) (EC 1.1.1.153) and, optionally, a GTP cyclohydrolase I (GCH1) (EC 3.5.4.16), and, optionally,
(b) a pterin-4-alpha-carbinolamine dehydratase (PCBD1) (EC 4.2.1.96); and, optionally, a dihydropteridine reductase (DHPR) (EC 1.5.1.34).

18. The recombinant microbial cell of claim 3, comprising
(a) a deletion or downregulation of an endogenous gene encoding an aromatic amino acid aminotransferase (EC 2.6.1.57), and/or
(b) a deletion or downregulation of an endogenous gene encoding a tryptophanase (EC 4.1.99.1).

19. The recombinant microbial cell of claim 3, which is derived from a microbial host cell which is a bacterial cell, a yeast host cell, a filamentous fungal cell, or an algal cell.
